(19) United States
(12) Patent Application Publication
(10) Pub. No.: US 2025/0241953 A1
(43) Pub. Date: Jul. 31, 2025

(54) TDT-SPECIFIC CHIMERIC RECEPTORS AND METHODS OF THEIR USE

(71) Applicant: Baylor College of Medicine, Houston, TX (US)

(72) Inventors: Maksim Mamonkin, Houston, TX (US); Norihiro Watanabe, Houston, TX (US)

(73) Assignee: Baylor College of Medicine, Houston, TX (US)

(21) Appl. No.: 18/859,252

(22) PCT Filed: Apr. 28, 2023
(86) PCT No.: PCT/US2023/066339

§ 371 Date: Oct. 23, 2024

Related U.S. Application Data
(60) Provisional application No. 63/336,892, filed on Apr. 29, 2022.

(51) Int. Cl. A61K 35/17; A61K 35/02; A61K 40/11; A61K 40/32; A61K 40/33; A61K 40/42; C07K 16/28; C07K 16/40; C12N 15/86

(52) U.S. Cl. CPC A61K 35/17; A61K 35/02; A61K 40/11; A61K 40/32; A61K 40/33; A61K 40/421; A61K 40/4244; C07K 16/2809; C07K 16/40; C12N 15/86; A61K 2239/27; A61K 2239/48; C07K 2317/31; C07K 2317/56; C07K 2317/622

(57) ABSTRACT

Embodiments of the disclosure concern methods and compositions related to targeted cancer therapy directed to a particular terminal deoxynucleotidyl transferase peptide associated with HLA-A02. In specific embodiments, cellular therapy employs cells encoding a chimeric T-cell receptor that targets the peptide and optionally also a bi-specific T-cell engager that targets the same peptide. In particular embodiments, one or both are used to treat a hematological malignancy.

This application claims priority to U.S. Provisional Patent Application Ser. No. 63/336,892, filed Apr. 29, 2022, which is incorporated by reference herein in its entirety.

### BACKGROUND

### I. Technical Field

This disclosure relates at least to the fields of cell biology, molecular biology, immunology, and medicine.

### II. Background

Chimeric antigen receptor (CAR) T cells targeting CD19 produce long-term remissions in less than half of patients with refractory/relapsed B-cell ALL. Antigen escape and limited therapeutic T-cell persistence hinder the clinical benefit of CD19-targeted approaches, prompting evaluation of combinatorial antigen targeting and structural optimization of chimeric receptors¹. On the other hand, continuous CD19 CAR T-cell activity often produces long-term systemic depletion of B-cells, requiring supplementation with IVIG. Targeted immunotherapy options are even more limited for T-cell ALL and AML, where potential on-target off-tumor toxicity may result in ablation of vital lymphocytes and primitive progenitors and dramatically increase the risks of these therapies². As a result, overall survival in patients with treatment-refractory or relapsed T-ALL and AML remains very poor^{2,3}.

The present disclosure satisfies a long-felt need in the art of providing effective adoptive cell therapy for cancer treatment.

### BRIEF SUMMARY

Embodiments of the disclosure encompass methods and compositions for treatment of medical conditions in which terminal deoxynucleotidyl transferase (TdT) is associated in any manner and in need of targeting. In specific embodiments, fragments of TdT are targeted as a course of action, including peptides of TdT. In particular embodiments, TdT peptides are the focus of treatment for an individual in need thereof. In particular embodiments, TdT peptides may or may not be associated with any member of the human leukocyte antigen (HLA) system, including at least HLA-A02, and may be the focus of treatment for an individual in need thereof. In specific cases, TdT peptide associated with HLA-A02 is targeted, including targeted on the surface of deleterious cells of any kind that express it. In some embodiments, the TdT peptide may be associated with any HLA antigen, including HLA-A, HLA-B, HLA-C, HLA-E, HLA-F or HLA-G.

In particular embodiments, an antibody that recognizes a portion of TdT, such as a TdT peptide, is utilized as a means of targeting cancer cells that express the peptide. The antibody may be utilized in any form, but in specific embodiments the antibody is utilized in a non-natural chimeric polypeptide of any kind, including a receptor and/or a multi-specific antibody. In some embodiments, the antibody is employed in a receptor as an extracellular portion so that the receptor may bind the TdT peptide as an antigen. In some embodiments, the antibody is utilized in a soluble protein, including one that may have one or more other antibodies directed to other antigen(s) that are not TdT peptide; in such cases, the protein may be a bi-specific antibody, tri-specific antibody, and so forth. In certain embodiments, cells expressing the chimeric receptor and/or the soluble antibody may be utilized, including as adoptive cell therapy, for example.

In various embodiments, the present disclosure addresses limitations in the art of cancer treatment by targeting TdT as a tumor antigen that is expressed on the surface of cancer cells of any kind, including at least highly expressed in both B- and T-cell acute lymphocytic leukemia (ALL) and in subsets of acute myeloid leukemia (AML) but absent in mature immune cells and early hematopoietic progenitors⁴ (FIG. 1). TdT is normally expressed in B- and T-cell precursors, where its activity has been linked with leukemogenesis^{5,6}; the presence of TdT in peripheral blasts is a common diagnostic criterion for leukemia. Therefore, targeting TdT enables a highly selective therapy of acute leukemia (for example) without the risk of off-tumor destruction of critical cell populations. This technology will solves limitations of adoptive cell therapy of leukemia. In other embodiments, solid tumors may be targeted by treatments encompassed herein.

It is contemplated that any embodiment discussed in this specification can be implemented with respect to any method or composition of the invention, and vice versa. Furthermore, compositions of the invention can be used to achieve methods of the invention.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1. TdT expression in normal and malignant lymphocytes.

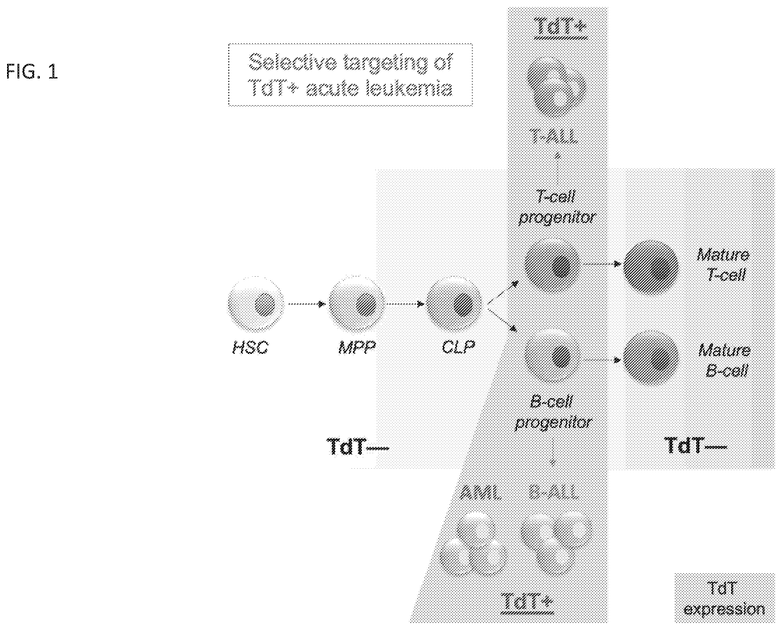

FIG. 2. Targeting TdT with chimeric TCR and BiTE.

FIGS. 3A-3E. Structure, expression, and function of TdT-specific CAR and cTCR. (FIG. 3A) A schematic showing CAR and cTCR structure. (FIG. 3B) Expression of TdT.CAR and cTCR in primary T-cells (n=7 donors). (FIG. 3C) Expansion of modified T-cells post-transduction (n=7). (FIG. 3D) Expression of CD27 and CD45RA on control and TdT-specific CD8+ T cells (n=6). (FIG. 3E) Cytotoxicity of TdT-specific T cells against BV173 cells upon a 3-day coculture at a 1:4 E:T ratio (n=3).

FIGS. 4A-4B. Specificity of TdT.cTCR T-cells. (FIG. 4A) Cytotoxicity of cTCR T cells against BV173 (TdT+/A2+), NALM6 (TdT+/A2+), CCRF-CEM (TdT+/A2−), and THP-1 (TdT−/A2+) cells upon a 3-day coculture. (n=3-4) (FIG. 4B) Viability of normal autologous T and B-cells after a coculture with TdT.cTCR-or CD19.CAR-expressing T-cells measured by Annexin V/7-AAD staining (n=3).

FIGS. 5A-5B. Effector function of TdT.cTCR-transduced CD4+ and CD8+ T-cells. Representative flow plots showing degranulation (FIG. 5A), and IFNγ/TNFα production (FIG. 5B) upon coculture with parental and MHC I-deficient (b2MKO) BV173 leukemia cells.

FIG. 6. Cytotoxicity of T cells redirected against TdT+/A2+ cell lines through CAR, cTCR, or BiTE. (n=3).

FIG. 7 illustrates one embodiment of a chimeric TCR in which V_{H} and V_{L} domains of the antibody are present on a single TCRα chain, in contrast to other receptors in the art (Liu et al., Sci Transl Med. 2021; 13(586):eabb5191). Murine constant chains of TCRa and TCRb are illustrated.

FIG. 8 represents cTCR and STAR expression on T cells after retroviral transduction. NT; non-transduced.

FIG. 9 demonstrates cytotoxicity of cTCR and STAR-expressing T cells against BV173 cell line (TdT+, HLA-A2+).

FIG. 10 shows cTCR and BiTE expression on T cells after retroviral transduction. NT; non-transduced.

FIG. 11 demonstrates cytotoxicity of cTCR T cells and BiTE-mediated killing against BV173 cell line (TdT+, HLA-A2+).

### DETAILED DESCRIPTION

### I. Examples of Definitions

Throughout this application, the term “about” is used to indicate that a value includes the inherent variation of error for the measurement or quantitation method.

The use of the word “a” or “an” when used in conjunction with the term “comprising” may mean “one.” but it is also consistent with the meaning of “one or more,” “at least one,” and “one or more than one.”

The phrase “and/or” means “and” or “or”. To illustrate, A, B, and/or C includes: A alone, B alone, C alone, a combination of A and B, a combination of A and C, a combination of B and C, or a combination of A, B, and C. In other words, “and/or” operates as an inclusive or.

As used herein, the phrase “at least one of,” when used with a list of items, means different combinations of one or more of the listed items may be used and only one of the items in the list may be needed. The item may be a particular object, thing, step, operation, process, or category. In other words, “at least one of” means any combination of items or number of items may be used from the list, but not all of the items in the list may be required. For example, without limitation, “at least one of item A, item B, or item C” means item A; item A and item B; item B; item A, item B, and item C; item B and item C; or item A and C. In some cases, “at least one of item A, item B, or item C” means, but is not limited to, two of item A, one of item B, and ten of item C; four of item B and seven of item C; or some other suitable combination.

The words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

Throughout this specification, unless the context requires otherwise, the words “comprise”, “comprises” and “comprising” will be understood to imply the inclusion of a stated step or element or group of steps or elements but not the exclusion of any other step or element or group of steps or elements. By “consisting of” is meant including, and limited to, whatever follows the phrase “consisting of.” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that no other elements are optional and may or may not be present depending upon whether or not they affect the activity or action of the listed elements.

Reference throughout this specification to “one embodiment,” “an embodiment,” “a particular embodiment,” “a related embodiment,” “a certain embodiment,” “an additional embodiment,” or “a further embodiment” or combinations thereof means that a particular feature, structure or characteristic described in connection with the embodiment is included in at least one embodiment of the present invention. Thus, the appearances of the foregoing phrases in various places throughout this specification are not necessarily all referring to the same embodiment. Furthermore, the particular features, structures, or characteristics may be combined in any suitable manner in various embodiments.

The term “peptide,” as used herein, generally refers to amino acids linked by peptide bonds. Peptides can include amino acid chains between about 5 and 50 residues. Peptides can include amino acid chains shorter than 10 residues, including, oligopeptides, dipeptides, tripeptides, and tetrapeptides. Peptides can include chains longer than 50 residues and may be referred to as “polypeptides” or “proteins.” In specific embodiments, the term refers to the TdT peptide of ALYDKTKRI (SEQ ID NO:3) or any peptide that is at least 70, 75, 80, 85, 86, 87, 88, 89, 90 or greater % identity to SEQ ID NO:3. The peptide may be at least 5, 6, 7, 8, or all of the contiguous residues of SEQ ID NO:3. There may be one or two substitutions compared to SEQ ID NO:3. In particular embodiments, any antibody encompassed herein may be able to target these variant peptides.

The term “subject,” as used herein, generally refers to an animal, such as a mammal (e.g., human). For example, the subject can include a vertebrate, a mammal, a rodent (e.g., a mouse), a primate, a simian, or a human. Animals may include, but are not limited to, farm animals, sport animals, and pets, including dogs, cats, and horses. A subject can include a healthy or asymptomatic individual, an individual that has or is suspected of having a disease (e.g., cancer) or a pre-disposition to the disease (such as being at greater risk than the general population), and/or an individual that is in need of therapy or suspected of needing therapy. A subject can be a patient. “Individual, “subject,” and “patient” are used interchangeably and can refer to a human or non-human.

“Treating” or treatment of a disease or condition refers to executing a protocol, which may include administering one or more drugs to an individual, such as a patient, in an effort to alleviate signs or symptoms of the disease. Desirable effects of treatment include decreasing the rate of disease progression, ameliorating or palliating the disease state, and remission or improved prognosis. Alleviation can occur prior to signs or symptoms of the disease or condition appearing, as well as after their appearance. Thus, “treating” or “treatment” may include “preventing” or “prevention” of disease or undesirable condition. In addition, “treating” or “treatment” does not require complete alleviation of signs or symptoms, does not require a cure, and specifically includes protocols that have only a marginal effect on the patient. The subject may or may not be receiving medical guidance via the internet.

The term “therapeutically effective” as used throughout this application refers to anything that promotes or enhances the well-being of the subject with respect to the medical treatment of this condition. This includes, but is not limited to, a reduction in the frequency and/or severity of one or more signs or symptoms of a disease and/or a delay in its onset or spreading, including cancer.

It is specifically contemplated that any limitation discussed with respect to one embodiment of the invention may apply to any other embodiment of the invention. Furthermore, any composition of the invention may be used in any method of the invention, and any method of the invention may be used to produce or to utilize any composition of the invention. Aspects of an embodiment set forth in the Examples are also embodiments that may be implemented in the context of embodiments discussed elsewhere in a different Example or elsewhere in the application, such as in the Brief Summary, Detailed Description, Claims, and Brief Description of the Drawings.

Any method in the context of a therapeutic, diagnostic, or physiologic purpose or effect may also be described in “use” claim language such as “Use of” any compound, composition, or agent discussed herein for achieving or implementing a described therapeutic, diagnostic, or physiologic purpose or effect.

In various embodiments, the methods and compositions enhance sensitivity of tumor antigen targeting by chimeric receptors. Active development of CAR- and TCR-based immune cell therapies and their clinical evaluation have prompted investigation of the mechanisms regulating tumor-specific receptor sensitivity and efficacy. While most CARs target antigens that are abundantly expressed on tumor cells, it is important to understand the limits of their antigen sensitivity and devise new strategies to overcome low responsiveness to tumors with reduced expression of the target antigen. Prior studies have suggested CARs have a significantly reduced sensitivity compared to TCRs and are prone to “ignoring” tumor variants with decreased surface density of the antigen^{7,8}. To overcome this limitation, certain embodiments combine high TCR sensitivity with the versatility of CAR-based targeting and provide a highly sensitive chimeric TCR and an antibody-derived antigen recognition moiety.

### II. Anti-TdT Peptide Antibody-Based Compositions

The present disclosure concerns treatment for one or more medical conditions using a specific antibody that targets one or more TdT peptides and compositions that utilize them. In various embodiments, a particular antibody is employed in one or more different compositions for targeting deleterious cells that express a TdT peptide, such as cancer cells that express a TdT peptide, and in specific embodiments the peptide comprises ALYDKTKRI (SEQ ID NO:3) or a variant thereof still recognized by the antibody. In some embodiments, one of the compositions that utilizes the antibody is cell-based and targets the TdT peptide, and the cell comprises a non-natural receptor on the surface of the cell that employs the antibody. In some embodiments, one of the compositions that utilizes the antibody is soluble and targets the TdT peptide, although the composition is outside of a cell (however, it may be delivered to an individual in the form of a cell following which the soluble protein comprising the antibody is secreted from the cell). In specific embodiments, both types of antibody-based compositions are utilized.

In particular embodiments, the disclosure includes antibodies that bind a part of the TdT protein, including that bind a TdT peptide comprising, consisting of, or consisting essentially of SEQ ID NO:3. Although the antibody may be of any kind, in specific embodiments the antibody comprises a single chain variable fragment (scFv).

In various embodiments, the disclosure provides multiple scFvs that may be utilized in any chimeric receptor, including any chimeric TCR and/or in any bi-specific or multi-specific antibody, including any bispecific T-cell engager (BiTE). In specific embodiments, the antibodies comprise the following representative sequences:

Clone 1: TdT_B1 Polynucleotide Sequence
(SEQ ID NO: 4)
ATGGCCTTACCAGTGACCGCCTTGCTCCTGCCGCTGGCCTTGCTGCTCCACGCCGCCAGG
CCGGCTCAAGTTCAGCTGGTGCAGAGCGGAGCGGAGGTGAAGAAGCCAGGGTCTTCCGT
TAAGGTCTCTTGTAAGGCCTCAGGCGGAACTTTCTCATCTTACGCTATTTCCTGGGTAAG
GCAAGCACCAGGACAGGGATTAGAATGGATGGGGGGGATTATCCCCATTTTCGGTACCG
CGAACTACGCCCAAAAATTTCAAGGTCGGGTAACCATCACCGCCGATGAATCGACATCT
ACCGCCTACATGGAACTGAGCTCTCTTCGGAGCGAGGACACCGCAGTCTATTACTGCGCG
AGAGACGGGTATAGTGGTAGCTACTACTATTATTACGGCATGGACGTTTGGGGCCAGGG
GACCTTAGTGACGGTGAGCAGCGGAGGAGGCGGCGGGTCCCAGTCTGCTCTGACCCAAC
CTGCCAGCGTCTCCGGCAGTCCAGGACAAAGTATCACCATCAGCTGCACTGGGACTTCCA
GCGATGTGGGGGGCTACAATTACGTAAGTTGGTACCAGCAACACCCTGGGAAGGCCCCC
AAGCTGATGATTTATGATGTCTCATACAGACCGAGCGGAGTGAGCCACCGTTTTAGTGGC
TCCAAGTCCGGAAACACCGCTTCACTAACAATCAGTGGCTTACAAGCAGAGGACGAAGC
CGATTATTACTGCTCATCTTACACCAGTTCCAGTACGCTTGTGTTTGGTACTGGAACAAA
GTTGACTGTGCTGGGC
Clone 1: TdT_B1 Polypeptide Sequence; signal peptide-V_{H}-V_{L}
(SEQ ID NO: 5)
MALPVTALLLPLALLLHAARPAQVQLVQSGAEVKKPGSSVKVSCKASGGTESSYAISWVRQ
APGQGLEWMGGIIPIFGTANYAQKFQGRVTITADESTSTAYMELSSLRSEDTAVYYCARDGY
SGSYYYYYGMDVWGQGTLVTVSSGGGGGSQSALTQPASVSGSPGQSITISCTGTSSDVGGYN
YVSWYQQHPGKAPKLMIYDVSYRPSGVSHRFSGSKSGNTASLTISGLQAEDEADYYCSSYTS
SSTLVFGTGTKLTVL, wherein the signal peptide is at the N-terminus
and lacks marking, the V_{H} domain is single underlined, and the V_{L}
domain is double underlined.

In specific embodiments for B1, the V_{H} and V_{L} are as follows:

V_{H}:
(SEQ ID NO: 22)
AQVQLVQSGAEVKKPGSSVKVSCKASGGTFSSYAISWVRQAPGQGLEWM
GGIIPIFGTANYAQKFQGRVTITADESTSTAYMELSSLRSEDTAVYYCA
RDGYSGSYYYYYGMDVWGQGTLVTVSS
V_{L}:
(SEQ ID NO: 25)
QSALTQPASVSGSPGQSITISCTGTSSDVGGYNYVSWYQQHPGKAPKLM
IYDVSYRPSGVSHRFSGSKSGNTASLTISGLQAEDEADYYCSSYTSSST
LVFGTGTKLTVL

In specific embodiments, the complementarity determining regions (CDRs) for B1 are as follows

HCDR1:
(SEQ ID NO: 28)
GGTFSSYA
HCDR2:
(SEQ ID NO: 29)
IIPIFGTA
HCDR3:
(SEQ ID NO: 30)
ARDGYSGSYYYYYGMDV
LCDR1:
(SEQ ID NO: 31)
SSDVGGYNY
LCDR2:
DVS
LCDR3:
(SEQ ID NO: 33)
SSYTSSSTLV
Clone 2: TdT_G7 Polynucleotide Sequence
(SEQ ID NO: 6)
ATGGCCTTACCAGTGACCGCCTTGCTCCTGCCGCTGGCCTTGCTGCTCCACGCCGCCAGG
CCGGCCCAAGTCCAGCTGGTTGAAAGCGGGGGAGGAGTGGTTCAGCCCGGAAGGAGTTT
GCGGTTGAGTTGTGCAGCATCAGGATTCACATTTTCTTCCTATGCAATGTCCTGGGTGCGT
CAGGCTCCCGGAAAGGGACTGGAATGGGTTAGCGCTATATCTGGCAGCGGAGACTCTAC
ATATTATGCTGATTCAGTCAAAGGCCGTTTCACCATCAGCAGAGACAACAGTAAGAATA
CCCTCTACCTTCAGATGAACTCGCTACGTGCTGAAGACACCGCCGTTTACTACTGTGCTA
AAGATCTCGACTCCTCAAGTCCGGACGATGCTTTTGACATCTGGGGACAAGGCACCACTG
TCACCGTCAGCAGCGGAGGCGCCGCGCTGGCCGAAGTGGCAGCCGCGGTGGCTGACCCT
ATCGTTCTGACTCAGTCTCCTGGAAAGCTGAGCCTCTCCCCAGGGGAGCGGGCCACTTTG
TCCTGTGGCGCGTCGCAGAGCGTATCATCCAATTACCTGGCATGGTACCAGCAGAAACCT
GGCCTGGCTCCTCGGCTTTTGATTTATGATGCTAGTATTAGGGCTACTGGCGTGCCTGATC
GGTTCTCGGGATCAGGGAGCGCGACTGACTTCACTTTGACGATCAGCCGGCTGGACCCTG
AGGATTTTGCGGTGTACTATTGCCACCAGTATTCATCTGCCCCCATGACTTTTGGACAGG
GCACTAAGCTGGAGATTAAGAGG
Clone 2: TdT_G7 Polypeptide Sequence
(SEQ ID NO: 7)
MALPVTALLLPLALLLHAARPAQVQLVESGGGVVQPGRSLRLSCAASGFTFSSYAMSWVRQ
APGKGLEWVSAISGSGDSTYYADSVKGRFTISRDNSKNTLYLQMNSLRAEDTAVYYCAKDL
DSSSPDDAFDIWGQGTTVTVSSGGAALAEVAAAVADPIVLTQSPGKLSLSPGERATLSCGASQ
SVSSNYLAWYQQKPGLAPRLLIYDASIRATGVPDRFSGSGSATDFTLTISRLDPEDFAVYYCH
QYSSAPMTFGQGTKLEIKR, wherein the signal peptide is at the N-terminus
and lacks marking, the V_{H} domain is single underlined, and the V_{L}
domain is double underlined.

In specific embodiments for G7, the V_{H} and V_{L} are as follows:

V_{H}:
(SEQ ID NO: 23)
AQVQLVESGGGVVQPGRSLRLSCAASGFTFSSYAMSWVRQAPGKGLEWV
SAISGSGDSTYYADSVKGRFTISRDNSKNTLYLQMNSLRAEDTAVYYCA
KDLDSSSPDDAFDIWGQGTTVTVSS
V_{L}:
(SEQ ID NO: 26)
AALAEVAAAVADPIVLTQSPGKLSLSPGERATLSCGASQSVSSNYLAWY
QQKPGLAPRLLIYDASIRATGVPDRFSGSGSATDFTLTISRLDPEDFAV
YYCHQYSSAPMTFGQGTKLEIKR

In specific embodiments, the complementarity determining regions (CDRs) for G7 are as follows

HCDR1:
(SEQ ID NO: 34)
GFTFSSYA
HCDR2:
(SEQ ID NO: 35)
ISGSGDST
HCDR3:
(SEQ ID NO: 36)
AKDLDSSSPDDAFDI
LCDR1:
(SEQ ID NO: 37)
QSVSSNY
LCDR2:
DAS
LCDR3:
(SEQ ID NO: 39)
HQYSSAPMT

Clone 3: TdT_G9 Polynucleotide Sequence
(SEQ ID NO: 8)
ATGGCCTTACCAGTGACCGCCTTGCTCCTGCCGCTGGCCTTGCTGCTCCACGCCGCCAGG
CCGGCTCAAGTGCAGCTGGTCGAATCTGGAGCTGGGACTAAGAAGCCTGGCGAGAGTCT
GAAGATCTCTTGTAAGGCCAGCGGCTACAACTTTGCTTCTTACTGGATCGGGTGGGTTAG
ACAGATGCCTGGAAAAGGACTTGAGTGGGTAGGGATCATTGATCCTAGCGATTCCGATA
CCAAATACAGCCCAAGCTTTGAAGGGCAAGTCACTATTAGTGTGGACAAGTCTATCAGC
ACTGCTCACCTGCAGTGGAGTAGCCTAGATGCGAGCGACACGGCCATGTACTACTGCGC
GAGGAGTCTGGGCAGCTACTATGGAGATTGGTATTTCGATCTCTGGGGGAGGGGCACAC
TGGTGACCGTGTCATCCGGAGGAGGCGGCGGGTCCAACTTTATGCTGACCCAGCCACATT
CAATGTCTGAAAGCCCTGGCAAAACCGTAACAATCTCGTGCACACGGTCAAGCGGGAGC
ATCGCCAACAACTATGTTCAGTGGTATCAGCAGAGACCCGGGAGTTCCCCTACCACTGTC
ATCTACGATGACAATCAGAGACCATCCGGGGTGCCCGACCGCTTCAGCGGCTCAATAGA
TAGCTCCAGCAACTCCGCCTCTTTAACAATCTCGGGCCTGAAAACCGAGGACGAAGCTG
ATTATTACTGCCAGTCCTATGACTCCAGCAACGTGATTTTTGGTGGAGGGACTAAACTGA
CCGTCCTCGGA
Clone 3: TdT_G9 Polypeptide Sequence
(SEQ ID NO: 9)
MALPVTALLLPLALLLHAARPAQVQLVESGAGTKKPGESLKISCKASGYNFASY
WIGWVRQMPGKGLEWVGIIDPSDSDTKYSPSFEGQVTISVDKSISTAHLOWSSLDASDTAMY
YCARSLGSYYGDWYFDLWGRGTLVTVSSGGGGGSNFMLTQPHSMSESPGKTVTISCTRSSGS
IANNYVQWYQQRPGSSPTTVIYDDNQRPSGVPDRFSGSIDSSSNSASLTISGLKTEDEADYYC
QSYDSSNVIFGGGTKLTVLG, wherein the signal peptide is at the N-
terminus and lacks marking, the V_{H} domain is single underlined,
and the V_{L} domain is double underlined.

In specific embodiments for G9, the V_{H} and V_{L} are as follows:

V_{H}:
(SEQ ID NO: 24)
AQVQLVESGAGTKKPGESLKISCKASGYNFASYWIGWVRQMPGKGLEWV
GIIDPSDSDTKYSPSFEGQVTISVDKSISTAHLOWSSLDASDTAMYYCA
RSLGSYYGDWYFDLWGRGTLVTVSS
V_{L}:
(SEQ ID NO: 27)
NEMLTQPHSMSESPGKTVTISCTRSSGSIANNYVQWYQQRPGSSPTTVI
YDDNQRPSGVPDRFSGSIDSSSNSASLTISGLKTEDEADYYCQSYDSSN
VIFGGGTKLTVLG

In specific embodiments, the complementarity determining regions (CDRs) for G9 are as follows

HCDR1:
(SEQ ID NO: 40)
GYNFASYW
HCDR2:
(SEQ ID NO: 41)
IDPSDSDT
HCDR3:
(SEQ ID NO: 42)
ARSLGSYYGDWYFDL
LCDR1:
(SEQ ID NO: 43)
SGSIANNY
LCDR2:
DDN
LCDR3:
(SEQ ID NO: 45)
QSYDSSNVI

The scFv in various embodiments comprises any one or more of SEQ ID NOs: 4-9. The scFv in specific embodiments comprises sequence that is at least 80, 85, 90, 91, 92, 93, 94, 95, 96, 97, 98, 99, or greater % identity to any one or more of SEQ ID NOs: 4-9.

Thus, in specific embodiments, any antibody utilized for any composition encompassed herein (a chimeric receptor, multi-specific antibody, etc.) may comprise one of the following V_{H} sequences or one or more modifications thereof:

(1)
(SEQ ID NO: 22)
AQVQLVQSGAEVKKPGSSVKVSCKASGGTFSSYAISWVRQAPGQGLEWM
GGIIPIFGTANYAQKFQGRVTITADESTSTAYMELSSLRSEDTAVYYCA
RDGYSGSYYYYYGMDVWGQGTLVTVSS;
(2)
(SEQ ID NO: 23)
AQVQLVESGGGVVQPGRSLRLSCAASGFTFSSYAMSWVRQAPGKGLEWV
SAISGSGDSTYYADSVKGRFTISRDNSKNTLYLQMNSLRAEDTAVYYCA
KDLDSSSPDDAFDIWGQGTTVTVSS;
or
(3)
(SEQ ID NO: 24)
AQVQLVESGAGTKKPGESLKISCKASGYNFASYWIGWVRQMPGKGLEWV
GIIDPSDSDTKYSPSFEGQVTISVDKSISTAHLQWSSLDASDTAMYYCA
RSLGSYYGDWYFDLWGRGTLVTVSS.

In specific embodiments, the V_{H} domain comprises SEQ ID NO:22, SEQ ID NO: 23, or SEQ ID NO: 24, or the V_{H} domain is at least 70, 75, 80, 85, 90, 91, 92, 93, 94, 95, 96, 97, 98, or 99% identical in sequence to SEQ ID NO:22, SEQ ID NO:23, or SEQ ID NO:24. The V_{H} domain may have 1, 2, 3, 4, 5, 6, 7, 8, 9, 10 or more modifications compared to SEQ ID NO:22, SEQ ID NO: 23, or SEQ ID NO: 24, including an amino acid substitution, an inversion, a deletion, and so forth. The N-terminal and/or C-terminal sequences may be truncated by 1, 2, 3, 4, 5, or more amino acids compared to SEQ ID NO: 22, SEQ ID NO:23, or SEQ ID NO:24. In specific embodiments, the V_{H} domain comprises at least 100, 105, 110, 115, 116, 117, 118, 119, 120, 121, 122, or all contiguous amino acids of SEQ ID NO: 22, SEQ ID NO: 23, or SEQ ID NO:24.

Thus, in specific embodiments, any antibody utilized for any composition encompassed herein (a chimeric receptor, multi-specific antibody, etc.) may comprise one of the following V_{L} sequences, or one or more modifications thereof:

(1)
(SEQ ID NO: 25)
QSALTQPASVSGSPGQSITISCTGTSSDVGGYNYVSWYQQHPGKAPKLM
IYDVSYRPSGVSHRFSGSKSGNTASLTISGLQAEDEADYYCSSYTSSST
LVFGTGTKLTVL;
(2)
(SEQ ID NO: 26)
AALAEVAAAVADPIVLTQSPGKLSLSPGERATLSCGASQSVSSNYLAWY
QQKPGLAPRLLIYDASIRATGVPDRFSGSGSATDFTLTISRLDPEDFAV
YYCHQYSSAPMTFGQGTKLEIKR;
or
(3)
(SEQ ID NO: 27)
NFMLTQPHSMSESPGKTVTISCTRSSGSIANNYVQWYQQRPGSSPTTVI
YDDNQRPSGVPDRFSGSIDSSSNSASLTISGLKTEDEADYYCQSYDSSN
VIFGGGTKLTVLG

In specific embodiments, the V_{L} domain comprises SEQ ID NO:25, SEQ ID NO:26, or SEQ ID NO:27, or the V_{L} domain is at least 70, 75, 80, 85, 90, 91, 92, 93, 94, 95, 96, 97, 98, or 99% identical in sequence to SEQ ID NO:25, SEQ ID NO: 26, or SEQ ID NO:27. The V_{L} domain may have 1, 2, 3, 4, 5, 6, 7, 8, 9, 10 or more modifications compared to SEQ ID NO:25, SEQ ID NO:26, or SEQ ID NO: 27, including an amino acid substitution, an inversion, a deletion, and so forth. The N-terminal and/or C-terminal sequences may be truncated by 1, 2, 3, 4, 5, or more amino acids compared to SEQ ID NO: 25, SEQ ID NO:26, or SEQ ID NO:27. In specific embodiments, the V_{H} domain comprises at least 100, 105, 110, 115, 116, 117, 118, 119, 120, 121, 122, or all contiguous amino acids of SEQ ID NO: 25, SEQ ID NO:26, or SEQ ID NO:27.

The scFv in various embodiments comprises any one or more of SEQ ID NOs: 4-9, 22-24, or 25-27. The scFv in specific embodiments comprises sequence that is at least 80, 85, 90, 91, 92, 93, 94, 95, 96, 97, 98, 99, or greater % identity to any one or more of SEQ ID NOs: 4-9, 22-24, or 25-27.

In particular embodiments, compositions encompassed herein may utilize an antibody or antigen binding fragment comprising a heavy chain variable region and a light chain variable region, wherein the heavy chain variable region comprises a HCDR1, HCDR2, and HCDR3 from a heavy chain variable region of an antibody clone from B1, G7, or G9 and wherein the light chain variable region comprises a LCDR1, LCDR2, and LCDR3 from the light chain variable region of the same respective clone of B1, G7, or G9. Further aspects relate to an antibody or antigen binding fragment comprising a heavy chain variable region and a light chain variable region, wherein the heavy chain variable region comprises a HCDR1, HCDR2, and HCDR3 having or having at least 80% sequence identity or having or having at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity with a HCDR1, HCDR2, and HCDR3 from a heavy chain variable region of an antibody clone of B1, G7, or G9, and wherein the light chain variable region comprises a LCDR1, LCDR2, and LCDR3 having or having at least least 80% sequence identity or having or having at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity with a LCDR1, LCDR2, and LCDR3 from the light chain variable region of the same respective clone of B1, G7, or G9.

In particular embodiments, compositions encompassed herein may utilize an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 28-30, respectively and the LCDR1, LCDR2, and LCDR3 comprises an amino acid sequence of SEQ ID NOS: 31, 33 and “DVS”, respectively.

In particular embodiments, compositions encompassed herein may utilize an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 34-36, respectively and the LCDR1, LCDR2, and LCDR3 comprises an amino acid sequence of SEQ ID NOS: 37, 39 or “DAS”, respectively.

In particular embodiments, compositions encompassed herein may utilize an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS: 40-42, respectively and the LCDR1, LCDR2, and LCDR3 comprises an amino acid sequence of SEQ ID NOS: 43, 45 or “DDN”, respectively.

Polynucleotides encoding the antibody or parts thereof, the antibody proteins, vectors that comprise the polynucleotide, vectors that encode the antibody proteins, and cells that encompass polynucleotides, proteins, and/or vectors are all encompassed herein. In particular embodiments, the antibody is utilized in a chimeric protein of any kind. In a particular embodiment, the chimeric protein is a receptor, including a receptor that binds a TdT peptide, such as a receptor that comprises an antibody that binds a TdT peptide. In a particular embodiment, the chimeric receptor is a chimeric T cell receptor (TCR) or a chimeric antigen receptor. Polynucleotides that encode the chimeric TCR comprising the antibody, the chimeric TCR protein, vectors that comprise the polynucleotide, vectors that encode the chimeric TCR protein, and cells that encompass polynucleotides, proteins, and/or vectors are all encompassed herein. The antibody portion of the chimeric TCR may be configured in a variety of ways with respect to the TCRα and β chains. Any vector may be utilized, including viral vectors and non-viral vectors. The viral vector may be an adenoviral vector, adeno-associated viral vector, retroviral vector, or lentiviral vector. A non-viral vector may be a plasmid or transposon.

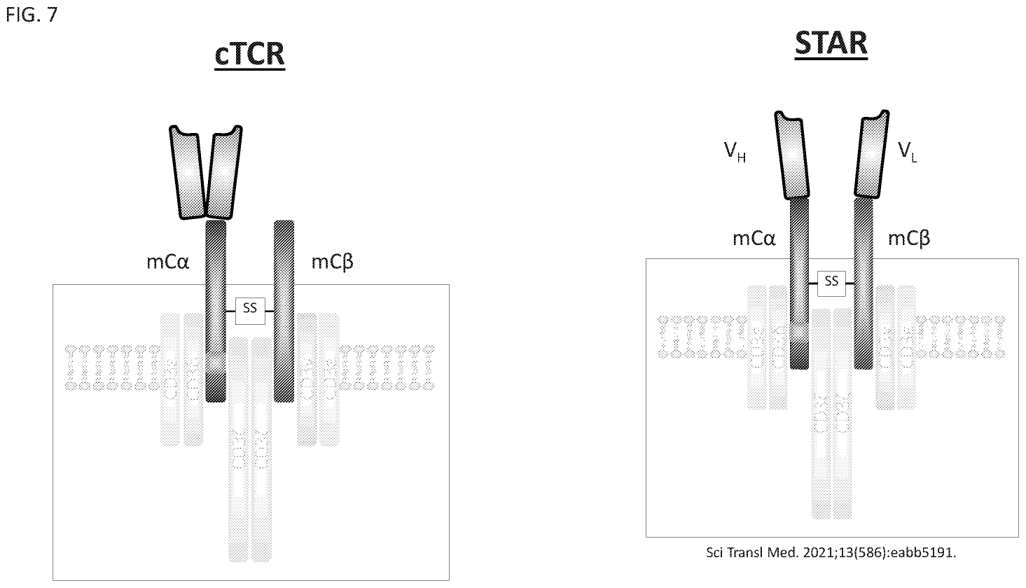

In one embodiment, the chimeric receptor is a synthetic T cell receptor (TCR) and antigen receptor (STAR) (Liu et al., Sci Transl Med. 2021; 13(586):eabb5191) that comprises one or more antigen-recognition domains of an antibody with constant regions of a TCR molecule (FIG. 7). In various embodiments, the STAR receptor is a double-chain TCRαβ-based receptor with variable regions of immunoglobulin heavy and light chains (V_{H} and V_{L}) fused to TCR-Cα and TCR-Cβ domains. In specific embodiments, a STAR receptor comprises a fusion of V_{H} and V_{L} to TCRα and β chain constant regions, respectively. In specific embodiments, a STAR receptor comprises a fusion of V_{L} and V_{H} to TCRα and β chain constant regions, respectively. Polynucleotides that encode the chimeric STAR comprising the antibody, the chimeric STAR protein, vectors that comprise the polynucleotide, vectors that encode the chimeric STAR protein, and cells that encompass polynucleotides, proteins, and/or vectors are all encompassed herein. Any vector may be utilized, including viral vectors and non-viral vectors. The viral vector may be an adenoviral vector, adeno-associated viral vector, retroviral vector, or lentiviral vector. A non-viral vector may be a plasmid or transposon.

Although one representative example of a STAR receptor that comprises the B1 antibody referred to above and that comprises two separate chains (the B1 STAR) is provided below, any antibody that binds TdT peptide may be utilized likewise with any STAR configuration.

B1_STAR (2 separate chains):
B1_STAR VH-TCRA Polynucleotide (the V_{H} domain of the B1 antibody
linked to the TCR alpha chain)
(SEQ ID NO: 10)
ATGGCCTTACCAGTGACCGCCTTGCTCCTGCCGCTGGCCTTGCTGCTCCACGCCGCCAGG
CCGGCTCAAGTTCAGCTGGTGCAGAGCGGAGCGGAGGTGAAGAAGCCAGGGTCTTCCGT
TAAGGTCTCTTGTAAGGCCTCAGGCGGAACTTTCTCATCTTACGCTATTTCCTGGGTAAG
GCAAGCACCAGGACAGGGATTAGAATGGATGGGGGGGATTATCCCCATTTTCGGTACCG
CGAACTACGCCCAAAAATTTCAAGGTCGGGTAACCATCACCGCCGATGAATCGACATCT
ACCGCCTACATGGAACTGAGCTCTCTTCGGAGCGAGGACACCGCAGTCTATTACTGCGCG
AGAGACGGGTATAGTGGTAGCTACTACTATTATTACGGCATGGACGTTTGGGGCCAGGG
GACCTTAGTGACGGTGAGCAGCGGATCCCCATACATTCAAAATCCAGAACCTGCGGTAT
ACCAATTGAAAGACCCAAGAAGCCAGGATTCCACGCTGTGTCTGTTTACAGACTTCGACT
CACAGATCAATGTTCCCAAGACAATGGAAAGCGGGACGTTCATCACCGATAAATGCGTT
CTGGACATGAAGGCTATGGATTCTAAAAGTAATGGTGCTATAGCCTGGAGTAACCAGAC
CTCATTTACTTGCCAAGACATTTTCAAGGAGACCAATGCTACATATCCATCCTCCGACGT
CCCTTGTGACGCCACTCTGACCGAGAAATCTTTTGAGACCGATATGAACCTGAACTTTCA
GAACCTACTGGTTATCGTTCTGAGAATACTGTTGCTCAAAGTCGCCGGATTCAACCTTCT
CATGACTCTGAGACTGTGGAGTAGC
B1_STAR VH-TCRA Polypeptide (the V_{H} domain of the B1 antibody linked
to the TCR alpha chain)
(SEQ ID NO: 11)
MALPVTALLLPLALLLHAARPAQVQLVQSGAEVKKPGSSVKVSCKASGGTFSSYAISWVRQ
APGQGLEWMGGIIPIFGTANYAQKFQGRVTITADESTSTAYMELSSLRSEDTAVYYCARDGY
SGSYYYYYGMDVWGQGTLVTVSSGSPYIQNPEPAVYQLKDPRSQDSTLCLFTDFDSQINVPK
TMESGTFITDKCVLDMKAMDSKSNGAIAWSNQTSFTCQDIFKETNATYPSSDVPCDATLTEK
SFETDMNLNFQNLLVIVLRILLLKVAGFNLLMTLRLWSS
B1_STAR VL-TCRB Polynucleotide (the V_{L} domain of the B1 antibody
linked to the TCR beta chain)
(SEQ ID NO: 12)
ATGGAGACAGACACACTCCTGctatgggtgctgctgctctGGGTTCCAGGTTCCACAGGTCAGTCTG
CTCTGACCCAACCTGCCAGCGTCTCCGGCAGTCCAGGACAAAGTATCACCATCAGCTGCA
CTGGGACTTCCAGCGATGTGGGGGGCTACAATTACGTAAGTTGGTACCAGCAACACCCT
GGGAAGGCCCCCAAGCTGATGATTTATGATGTCTCATACAGACCGAGCGGAGTGAGCCA
CCGTTTTAGTGGCTCCAAGTCCGGAAACACCGCTTCACTAACAATCAGTGGCTTACAAGC
AGAGGACGAAGCCGATTATTACTGCTCATCTTACACCAGTTCCAGTACGCTTGTGTTTGG
TACTGGAACAAAGTTGACTGTGCTGGGCACGCGTGAGGACTTGAGGAATGTGACGCCAC
CGAAGGTCTCTTTGTTTGAGCCGTCTAAGGCAGAAATTGCTAATAAACAAAAGGCCACCC
TAGTGTGTCTCGCGAGGGGATTCTTTCCTGATCACGTTGAGTTGTCCTGGTGGGTGAATG
GTAAGGAGGTTCATAGCGGTGTCTGCACCGACCCCCAGGCATACAAGGAGTCGAACTAC
TCCTACTGCCTGTCATCTAGACTCCGAGTCAGTGCCACGTTCTGGCATAACCCCCGTAAC
CATTTTAGGTGCCAGGTGCAATTCCACGGGCTTAGTGAAGAGGACAAGTGGCCAGAGGG
GTCCCCAAAGCCGGTGACCCAAAATATCTCTGCGGAAGCTTGGGGGCGCGCCGATTGTG
GGATTACCAGCGCGAGCTATCAGCAGGGAGTCCTGAGTGCAACGATACTCTACGAGATA
TTACTGGGAAAGGCTACATTATATGCAGTGTTGGTGAGCACTCTGGTCGTCATGGCAATG
GTTAAGAGAAAGAATAGCTAA
B1_STAR VL-TCRB Polypeptide (the VL domain of the B1 antibody linked
to the TCR beta chain)
(SEQ ID NO: 13)
METDTLLLWVLLLWVPGSTGQSALTQPASVSGSPGQSITISCTGTSSDVGGYNYV
SWYQQHPGKAPKLMIYDVSYRPSGVSHRFSGSKSGNTASLTISGLQAEDEADYYCSSYTSSST
LVFGTGTKLTVLGTREDLRNVTPPKVSLFEPSKAEIANKQKATLVCLARGFFPDHVELSWWV
NGKEVHSGVCTDPQAYKESNYSYCLSSRLRVSATFWHNPRNHFRCQVQFHGLSEEDKWPEG
SPKPVTQNISAEAWGRADCGITSASYQQGVLSATILYEILLGKATLYAVLVSTLVVMAMVKR
KNS

In one embodiment, the chimeric receptor is a chimeric T-cell receptor (cTCR) that comprises specific domains or sequences. In such cases, an scFv is fused to the TCRα and/or the TCRβ chains. In a specific embodiment, both of the VH and VL domains are linked to a TCR alpha chain or both of the VH and VL domains are linked to a TCR beta chain. Although one representative example of a cTCR comprises the B1 antibody referred to above linked to TCRα or β chains (B1 cTCR), any antibody that binds TdT peptide may be utilized likewise with any configuration of TCRαβ chains. In cases wherein the V_{L} and V_{H} domains of an antibody are linked to the TCR alpha chain, the TCR beta chain would be considered “empty” (e.g., lacking the variable domain sequences). In cases wherein the V_{L} and V_{H} domains of an antibody are linked to the TCR beta chain, the TCR alpha chain would be considered “empty” (e.g., lacking the variable domain sequences).

B1.cTCR:
B1_cTCR VHVL-TCRA Polynucleotide (the V_{L} and V_{H} domains of the B1
antibody linked to the TCR alpha chain)
(SEQ ID NO: 14)
ATGGCCTTACCAGTGACCGCCTTGCTCCTGCCGCTGGCCTTGCTGCTCCACGC
CGCCAGGCCGGCTCAAGTTCAGCTGGTGCAGAGCGGAGCGGAGGTGAAGAAGCCAGGG
TCTTCCGTTAAGGTCTCTTGTAAGGCCTCAGGCGGAACTTTCTCATCTTACGCTATTTCCT
GGGTAAGGCAAGCACCAGGACAGGGATTAGAATGGATGGGGGGGATTATCCCCATTTTC
GGTACCGCGAACTACGCCCAAAAATTTCAAGGTCGGGTAACCATCACCGCCGATGAATC
GACATCTACCGCCTACATGGAACTGAGCTCTCTTCGGAGCGAGGACACCGCAGTCTATTA
CTGCGCGAGAGACGGGTATAGTGGTAGCTACTACTATTATTACGGCATGGACGTTTGGGG
CCAGGGGACCTTAGTGACGGTGAGCAGCGGAGGAGGCGGCGGGTCCCAGTCTGCTCTGA
CCCAACCTGCCAGCGTCTCCGGCAGTCCAGGACAAAGTATCACCATCAGCTGCACTGGG
ACTTCCAGCGATGTGGGGGGCTACAATTACGTAAGTTGGTACCAGCAACACCCTGGGAA
GGCCCCCAAGCTGATGATTTATGATGTCTCATACAGACCGAGCGGAGTGAGCCACCGTTT
TAGTGGCTCCAAGTCCGGAAACACCGCTTCACTAACAATCAGTGGCTTACAAGCAGAGG
ACGAAGCCGATTATTACTGCTCATCTTACACCAGTTCCAGTACGCTTGTGTTTGGTACTGG
AACAAAGTTGACTGTGCTGGGCGGATCCCCATACATTCAAAATCCAGAACCTGCGGTAT
ACCAATTGAAAGACCCAAGAAGCCAGGATTCCACGCTGTGTCTGTTTACAGACTTCGACT
CACAGATCAATGTTCCCAAGACAATGGAAAGCGGGACGTTCATCACCGATAAATGCGTT
CTGGACATGAAGGCTATGGATTCTAAAAGTAATGGTGCTATAGCCTGGAGTAACCAGAC
CTCATTTACTTGCCAAGACATTTTCAAGGAGACCAATGCTACATATCCATCCTCCGACGT
CCCTTGTGACGCCACTCTGACCGAGAAATCTTTTGAGACCGATATGAACCTGAACTTTCA
GAACCTACTGGTTATCGTTCTGAGAATACTGTTGCTCAAAGTCGCCGGATTCAACCTTCT
CATGACTCTGAGACTGTGGAGTAGC
B1_cTCR VHVL-TCRA Polypeptide (the V_{L} and V_{H} domains of the B1 antibody
linked to the TCR alpha chain)
(SEQ ID NO: 15)
MALPVTALLLPLALLLHAARPAQVQLVQSGAEVKKPGSSVKVSCKASGGTFSSY
AISWVRQAPGQGLEWMGGIIPIFGTANYAQKFQGRVTITADESTSTAYMELSSLRSEDTAVY
YCARDGYSGSYYYYYGMDVWGQGTLVTVSSGGGGGSQSALTQPASVSGSPGQSITISCTGTS
SDVGGYNYVSWYQQHPGKAPKLMIYDVSYRPSGVSHRFSGSKSGNTASLTISGLQAEDEAD
YYCSSYTSSSTLVFGTGTKLTVLGGSPYIQNPEPAVYQLKDPRSQDSTLCLFTDFDSQINVPKT
MESGTFITDKCVLDMKAMDSKSNGAIAWSNQTSFTCQDIFKETNATYPSSDVPCDATLTEKS
FETDMNLNFQNLLVIVLRILLLKVAGFNLLMTLRLWSS
B1_cTCR VHVL-TCRB Polynucleotide (the V_{L} and V_{H} domains of the B1
antibody linked to the TCR beta chain)
(SEQ ID NO: 16)
ATGGCCTTACCAGTGACCGCCTTGCTCCTGCCGCTGGCCTTGCTGCTCCACGC
CGCCAGGCCGGCTCAAGTTCAGCTGGTGCAGAGCGGAGCGGAGGTGAAGAAGCCAGGG
TCTTCCGTTAAGGTCTCTTGTAAGGCCTCAGGCGGAACTTTCTCATCTTACGCTATTTCCT
GGGTAAGGCAAGCACCAGGACAGGGATTAGAATGGATGGGGGGGATTATCCCCATTTTC
GGTACCGCGAACTACGCCCAAAAATTTCAAGGTCGGGTAACCATCACCGCCGATGAATC
GACATCTACCGCCTACATGGAACTGAGCTCTCTTCGGAGCGAGGACACCGCAGTCTATTA
CTGCGCGAGAGACGGGTATAGTGGTAGCTACTACTATTATTACGGCATGGACGTTTGGGG
CCAGGGGACCTTAGTGACGGTGAGCAGCGGAGGAGGCGGCGGGTCCCAGTCTGCTCTGA
CCCAACCTGCCAGCGTCTCCGGCAGTCCAGGACAAAGTATCACCATCAGCTGCACTGGG
ACTTCCAGCGATGTGGGGGGCTACAATTACGTAAGTTGGTACCAGCAACACCCTGGGAA
GGCCCCCAAGCTGATGATTTATGATGTCTCATACAGACCGAGCGGAGTGAGCCACCGTTT
TAGTGGCTCCAAGTCCGGAAACACCGCTTCACTAACAATCAGTGGCTTACAAGCAGAGG
ACGAAGCCGATTATTACTGCTCATCTTACACCAGTTCCAGTACGCTTGTGTTTGGTACTGG
AACAAAGTTGACTGTGCTGGGCGGATCCACGCGTGAGGACTTGAGGAATGTGACGCCAC
CGAAGGTCTCTTTGTTTGAGCCGTCTAAGGCAGAAATTGCTAATAAACAAAAGGCCACCC
TAGTGTGTCTCGCGAGGGGATTCTTTCCTGATCACGTTGAGTTGTCCTGGTGGGTGAATG
GTAAGGAGGTTCATAGCGGTGTCTGCACCGACCCCCAGGCATACAAGGAGTCGAACTAC
TCCTACTGCCTGTCATCTAGACTCCGAGTCAGTGCCACGTTCTGGCATAACCCCCGTAAC
CATTTTAGGTGCCAGGTGCAATTCCACGGGCTTAGTGAAGAGGACAAGTGGCCAGAGGG
GTCCCCAAAGCCGGTGACCCAAAATATCTCTGCGGAAGCTTGGGGGCGCGCCGATTGTG
GGATTACCAGCGCGAGCTATCAGCAGGGAGTCCTGAGTGCAACGATACTCTACGAGATA
TTACTGGGAAAGGCTACATTATATGCAGTGTTGGTGAGCACTCTGGTCGTCATGGCAATG
GTTAAGAGAAAGAATAGCTAA
B1_cTCR VHVL-TCRB Polypeptide (the V_{L} and V_{H} domains of the B1 antibody
linked to the TCR beta chain)
(SEQ ID NO: 17)
MALPVTALLLPLALLLHAARPAQVQLVQSGAEVKKPGSSVKVSCKASGGTFSSY
AISWVRQAPGQGLEWMGGIIPIFGTANYAQKFQGRVTITADESTSTAYMELSSLRSEDTAVY
YCARDGYSGSYYYYYGMDVWGQGTLVTVSSGGGGGSQSALTQPASVSGSPGQSITISCTGTS
SDVGGYNYVSWYQQHPGKAPKLMIYDVSYRPSGVSHRFSGSKSGNTASLTISGLQAEDEAD
YYCSSYTSSSTLVFGTGTKLTVLGGSTREDLRNVTPPKVSLFEPSKAEIANKQKATLVCLARG
FFPDHVELSWWVNGKEVHSGVCTDPQAYKESNYSYCLSSRLRVSATFWHNPRNHFRCQVQF
HGLSEEDKWPEGSPKPVTQNISAEAWGRADCGITSASYQQGVLSATILYEILLGKATLYAVLV
STLVVMAMVKRKNS.

One example of a TCR beta chain considered “empty” (lacking any antibody material) is as follows and may be combined in a cell with a TCR alpha chain itself linked to V_{H} and V_{L} domains:

TCRb_empty
(SEQ ID NO: 20)
ATGGAGACAGACACACTCCTGctatgggtgctgctgctctgggttCCAGGTTCCACAGGTGGCGGCGGT
GGGTCAGAACAAAAATTGATTTCAGAGGAAGATCTCGGAGGGGGAGGGTCAACGCGTG
AGGACTTGAGGAATGTGACGCCACCGAAGGTCTCTTTGTTTGAGCCGTCTAAGGCAGAA
ATTGCTAATAAACAAAAGGCCACCCTAGTGTGTCTCGCGAGGGGATTCTTTCCTGATCAC
GTTGAGTTGTCCTGGTGGGTGAATGGTAAGGAGGTTCATAGCGGTGTCTGCACCGACCCC
CAGGCATACAAGGAGTCGAACTACTCCTACTGCCTGTCATCTAGACTCCGAGTCAGTGCC
ACGTTCTGGCATAACCCCCGTAACCATTTTAGGTGCCAGGTGCAATTCCACGGGCTTAGT
GAAGAGGACAAGTGGCCAGAGGGGTCCCCAAAGCCGGTGACCCAAAATATCTCTGCGGA
AGCTTGGGGGCGCGCCGATTGTGGGATTACCAGCGCGAGCTATCAGCAGGGAGTCCTGA
GTGCAACGATACTCTACGAGATATTACTGGGAAAGGCTACATTATATGCAGTGTTGGTGA
GCACTCTGGTCGTCATGGCAATGGTTAAGAGAAAGAATAGCTAA
TCRb_empty protein
(SEQ ID NO: 21)
METDTLLLWVLLLWVPGSTGGGGGSEQKLISEEDLGGGGSTREDLRNVTPPKVS
LFEPSKAEIANKQKATLVCLARGFFPDHVELSWWVNGKEVHSGVCTDPQAYKESNYSYCLS
SRLRVSATFWHNPRNHFRCQVQFHGLSEEDKWPEGSPKPVTQNISAEAWGRADCGITSASYQ
QGVLSATILYEILLGKATLYAVLVSTLVVMAMVKRKNS

Representative sequences of TCR alpha chains that are “empty” (lacking any antibody material) are known in the art.

In one embodiment, there are methods and compositions that utilize a bi-specific T cell engager of a specific sequence that comprises any antibody that binds a TdT peptide. Although a specific example of an engager utilizes the B1 antibody clone referred to above, in other embodiments any TdT-specific antibody is employed in a BiTE.

TdT B1 BiTE Polynucleotide (B1 antibody linked to anti-CD3 antibody)
(SEQ ID NO: 18)
ATGGATTGGATCTGGCGCATCCTGTTTCTCGTGGGAGCCGCCACAGGC
GCCCATTCTGCTCAAGTTCAGCTGGTGCAGAGCGGAGCGGAGGTGAAGAAGCCA
GGGTCTTCCGTTAAGGTCTCTTGTAAGGCCTCAGGCGGAACTTTCTCATCTTACG
CTATTTCCTGGGTAAGGCAAGCACCAGGACAGGGATTAGAATGGATGGGGGGGA
TTATCCCCATTTTCGGTACCGCGAACTACGCCCAAAAATTTCAAGGTCGGGTAAC
CATCACCGCCGATGAATCGACATCTACCGCCTACATGGAACTGAGCTCTCTTCGG
AGCGAGGACACCGCAGTCTATTACTGCGCGAGAGACGGGTATAGTGGTAGCTAC
TACTATTATTACGGCATGGACGTTTGGGGCCAGGGGACCTTAGTGACGGTGAGC
AGCGGAGGAGGCGGCGGGTCCCAGTCTGCTCTGACCCAACCTGCCAGCGTCTCC
GGCAGTCCAGGACAAAGTATCACCATCAGCTGCACTGGGACTTCCAGCGATGTG
GGGGGCTACAATTACGTAAGTTGGTACCAGCAACACCCTGGGAAGGCCCCCAAG
CTGATGATTTATGATGTCTCATACAGACCGAGCGGAGTGAGCCACCGTTTTAGTG
GCTCCAAGTCCGGAAACACCGCTTCACTAACAATCAGTGGCTTACAAGCAGAGG
ACGAAGCCGATTATTACTGCTCATCTTACACCAGTTCCAGTACGCTTGTGTTTGGT
ACTGGAACAAAGTTGACTGTGCTGGGCTCCGGAGGTGGTGGATCCGATATCAAA
CTGCAGCAGTCAGGGGCTGAACTGGCAAGACCTGGGGCCTCAGTGAAGATGTCC
TGCAAGACTTCTGGCTACACCTTTACTAGGTACACGATGCACTGGGTAAAACAGA
GGCCTGGACAGGGTCTGGAATGGATTGGATACATTAATCCTAGCCGTGGTTATAC
TAATTACAATCAGAAGTTCAAGGACAAGGCCACATTGACTACAGACAAATCCTC
CAGCACAGCCTACATGCAACTGAGCAGCCTGACATCTGAGGACTCTGCAGTCTAT
TACTGTGCAAGATATTATGATGATCATTACTGCCTTGACTACTGGGGCCAAGGCA
CCACTCTCACAGTCTCCTCAGGTGGTGGTGGTTCTGGCGGCGGCGGCTCCGGTGG
TGGTGGTTCTGACATTCAGCTGACCCAGTCTCCAGCAATCATGTCTGCATCTCCA
GGGGAGAAGGTCACCATGACCTGCAGAGCCAGTTCAAGTGTAAGTTACATGAAC
TGGTACCAGCAGAAGTCAGGCACCTCCCCCAAAAGATGGATTTATGACACATCC
AAAGTGGCTTCTGGAGTCCCTTATCGCTTCAGTGGCAGTGGGTCTGGGACCTCAT
ACTCTCTCACAATCAGCAGCATGGAGGCTGAAGATGCTGCCACTTATTACTGCCA
ACAGTGGAGTAGTAACCCGCTCACGTTCGGTGCTGGGACCAAGCTGGAGCTGAA
ATCCTAA
TdT B1 BiTE Polypeptide (B1 antibody linked to anti-CD3 antibody)
(SEQ ID NO: 19)
MDWIWRILFLVGAATGAHSAQVQLVQSGAEVKKPGSSVKVSCKASGGTF
SSYAISWVRQAPGQGLEWMGGIIPIFGTANYAQKFQGRVTITADESTSTAYMELSSLR
SEDTAVYYCARDGYSGSYYYYYGMDVWGQGTLVTVSSGGGGGSQSALTQPASVSG
SPGQSITISCTGTSSDVGGYNYVSWYQQHPGKAPKLMIYDVSYRPSGVSHRFSGSKS
GNTASLTISGLQAEDEADYYCSSYTSSSTLVFGTGTKLTVLGSGGGGSDIKLQQSGAE
LARPGASVKMSCKTSGYTFTRYTMHWVKQRPGQGLEWIGYINPSRGYTNYNQKFK
DKATLTTDKSSSTAYMQLSSLTSEDSAVYYCARYYDDHYCLDYWGQGTTLTVSSG
GGGSGGGGSGGGGSDIQLTQSPAIMSASPGEKVTMTCRASSSVSYMNWYQQKSGTS
PKRWIYDTSKVASGVPYRFSGSGSGTSYSLTISSMEAEDAATYYCQQWSSNPLTFGA
GTKLELKS

In various embodiments, any polynucleotide that comprises sequence that encodes a chimeric receptor protein (chimeric TCR or STAR or chimeric antigen receptor) and/or a BiTE may also encode one or more other gene products. In particular embodiments, the polynucleotide that encodes the chimeric receptor protein may also encode another gene product that also uses an antibody, including any anti-TdT peptide antibody. In specific embodiments, the polynucleotide that encodes the chimeric receptor protein further comprises sequence that encodes the bispecific T-cell engager (BiTE), including one that comprises the anti-TdT peptide antibody. In specific cases, the polynucleotide that encodes the TCR protein is a different polynucleotide than one that encodes the BiTE that comprises an antibody specific for TdT or a TdT peptide.

In specific embodiments, the disclosure concerns TdT-specific cytotoxic receptors and methods of their use. This disclosure encompasses novel reagents that recognize and eliminate cancer cells of any kind (including hematological cancers, such as acute leukemia cells, or solid tumors) without producing substantial damage to normal cells. In specific embodiments, the disclosure includes a T-cell-based (chimeric T-cell receptor (TCR) or STAR) and/or a recombinant protein-based (bispecific T-cell engager) reagent to target a specific TdT peptide, including one presented on the cell surface, such as in the context of HLA-A2. Potent activity of TdT-redirected T-cells in preclinical models of human acute leukemia is demonstrated herein, although in other embodiments immune effector cells other than T cells are utilized in the adoptive cell therapy.

Embodiments of the disclosure allow for redirecting of both CD4+ and CD8+ T-cells against an MHC class I-presented tumor antigen. T-cells expressing naturally-occurring TdT-specific TCRs are deleted during thymic selection, thus precluding selection or expansion of natural TdT-specific T cells in patients in need of them, such as with leukemia. To overcome this limitation, the inventors identified an antibody binder that specifically recognizes a dominant TdT peptide in the context of HLA-A02, the most common MHC class I allele in the Western world. Because this binding does not require the CD8 coreceptor, expression of a TdT-specific cTCR effectively redirects both CD8+ and CD4+ T-cells to elicit effector functions against TdT+ leukemia, unlike conventional MHC I-specific TCRs that only activate CD8+ T-cells. Based on the current preclinical and clinical evidence, CD4+ T-cell activation improves TdT-specific T-cell responses and overall anti-leukemic activity, in specific embodiments.

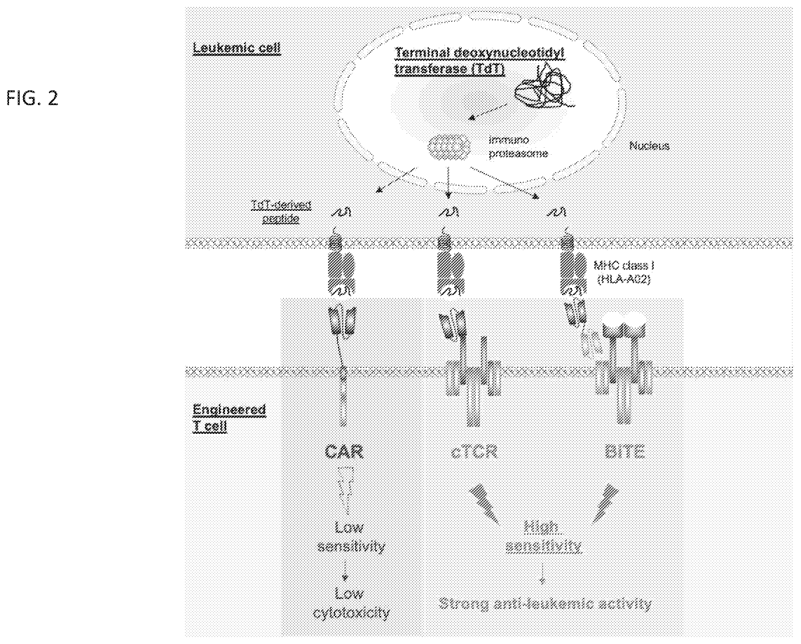

As demonstrated elsewhere herein, T cells expressing a TdT-specific cTCR have more potent anti-leukemic activity compared to those expressing an analogous TdT-specific chimeric antigen receptor (CAR). These results indicate that in various embodiments optimal recognition of rare TdT/HLA complexes requires highly sensitive TCR signaling. As shown herein, unmodified T-cells can be effectively redirected against TdT+ leukemia in the presence of soluble TdT-specific T-cell engagers that similarly activates TCR signaling in bystander, nonmodified T-cells. These results provide the scientific basis for developing both cellular and acellular strategies to target TdT using cTCR-transgenic T-cells or recombinant bispecific engagers, respectively. Compared to autologous engineered T-cells, bispecific engagers offer off-the-shelf availability and reduced manufacturing cost, and their application in the course of treatment, including early in the course of treatment, may improve antitumor activity of standard of care therapy, especially in patients with high-risk T-ALL and AML (FIG. 2). Thus, in specific embodiments of the disclosure, there are methods and compositions that employ adoptive T-cell therapy and bispecific T-cell engagers for the same individual.

In various embodiments, methods and compositions concern a recombinant T-cell receptor (TCR) (that uses TCRα and β chains, including cTCR referred to herein and also to STARs referred to herein) comprising a single-chain fragment variable (scFv) specific for a terminal deoxynucleotidyl transferase (TdT) or a TdT peptide. Any embodiments associated with the receptor may be utilized, including the non-natural TCR polypeptide, recombinant polynucleotides that encode the TCR polypeptide, cells (including those isolated from nature, commercially obtained, obtained from a donor or an individual to be treated with them, etc.) that comprise the TCR polypeptide or polynucleotides that encode the TCR polypeptide, vector polynucleotides of any kind that express the TCR polypeptide, cells that harbor one or more recombinant polynucleotides, and so forth. In particular embodiments of the recombinant TCR, the TCR comprises a binder, such as an antibody, that targets a TdT peptide, including one that may be associated with HLA-A02 on cell surfaces (including cancer cell surfaces, as opposed to non-cancerous cells).

The TCR is non-natural for having been engineered by the hand of man to include an antibody that binds a TdT peptide and in some embodiments structurally comprises a disulfide-linked membrane-anchored heterodimeric protein comprising α chain and β chain, and the TCR is in a configuration not found in nature. In specific embodiments, a TCRα chain and/or TCRβ chain in the recombinant TCR have one or more modifications by the hand of man, such as to prevent cross-pairing with endogenous TCR when present in a cell and/or to enhance heterologous pairing with each other. In specific embodiments, the α chain and/or β chain are not from a human but are from another mammal, such as a mouse, rat, or monkey, for example. In specific embodiments, both V_{L} and V_{H} domains of the anti-TdT peptide antibody are attached to the same TCR chain (α or β) (FIG. 7). In some embodiments, the respective TCR chains are attached to either V_{H} or V_{L} of the anti-TdT peptide antibody. In specific embodiments, representative sequences of the TCR alpha and TCR beta chains may be employed in the compositions of the disclosure, where the bold underlined sequences have been modified compared to wild-type:

TCR alpha:
(SEQ ID NO: 1)
PYIQNPEPAVYQLKDPRSQDSTLCLFTDFDSQINVPKTMESGTFITDKC
VLDMKAMDSKSNGAIAWSNQTSFTCQDIFKETNATYPSSDVPCDATLTE
KSFETDMNLNFQNLLVIVLRILLLKVAGFNLLMTLRLWSS
TCR beta:
(SEQ ID NO: 2)
EDLRNVTPPKVSLFEPSKAEIANKQKATLVCLARGFFPDHVELSWWVNG
KEVHSGVCTDPQAYKESNYSYCLSSRLRVSATFWHNPRNHFRCQVQFHG
LSEEDKWPEGSPKPVTQNISAEAWGRADCGITSASYQQGVLSATILYEI
LLGKATLYAVLVSTLVVMAMVKRKNS

Any protein, polynucleotide, and/or cells encompassed herein may be comprised in a composition. Cells in a composition may be of any kind, including immune cells, such as immune effector cells. The cells in a composition may be T cells, NK cells, NK T cells, B cells, macrophages, neutrophils, or a combination thereof. When the cell are T cells, the T cells may be αβ T cells, γδ T cells, T-helper cells, invariant natural killer T (iNKT) cells, cytotoxic T cells, T-regulatory cells natural-killer (NK) cells, or a combination thereof. In various embodiments, the cells may comprise (a) a polynucleotide comprising (1) sequence that encodes a recombinant TCR comprising an scFv specific for TdT or a TdT peptide; and (2) sequence that encodes a BiTE comprising an antibody specific for TdT or a TdT peptide; or (b) a first polynucleotide comprising sequence that encodes a recombinant TCR comprising an scFv specific for TdT or a TdT peptide; and a second polynucleotide comprising sequence that encodes a BiTE comprising an antibody specific for TdT or a TdT peptide.

In some embodiments, the chimeric receptor that is utilized may include a chimeric antigen receptor (CAR). The CAR may be first generation, second generation, third generation, and so on. In specific embodiments, the antibody portion of the CAR targets the TdT peptide which may or may not be associated with an HLA. The hinge, transmembrane domain, CD3zeta (or a domain with a similar function), and optionally one or more costimulatory domains may be of any suitable kind.

### III. Antibodies, Generally

Aspects of the disclosure relate to anti-TdT antibodies or fragments thereof. The term “antibody” refers to an intact immunoglobulin of any isotype, or a fragment thereof that can compete with the intact antibody for specific binding to the target antigen, and includes chimeric, humanized, fully human, and bispecific antibodies. As used herein, the terms “antibody” or “immunoglobulin” are used interchangeably and refer to any of several classes of structurally related proteins that function as part of the immune response of an animal, including IgG, IgD, IgE, IgA, IgM, and related proteins, as well as polypeptides comprising antibody CDR domains that retain antigen-binding activity.

The term “antigen” refers to a molecule or a portion of a molecule capable of being bound by a selective binding agent, such as an antibody. An antigen may possess one or more epitopes that are capable of interacting with different antibodies.

The term “epitope” includes any region or portion of molecule capable eliciting an immune response by binding to an immunoglobulin or to a T-cell receptor. Epitope determinants may include chemically active surface groups such as amino acids, sugar side chains, phosphoryl or sulfonyl groups, and may have specific three-dimensional structural characteristics and/or specific charge characteristics. Generally, antibodies specific for a particular target antigen will preferentially recognize an epitope on the target antigen within a complex mixture.

The epitope regions of a given polypeptide can be identified using many different epitope mapping techniques are well known in the art, including: x-ray crystallography, nuclear magnetic resonance spectroscopy, site-directed mutagenesis mapping, protein display arrays, see, e.g., Epitope Mapping Protocols, (Johan Rockberg and Johan Nilvebrant, Ed., 2018) Humana Press, New York, N.Y. Such techniques are known in the art and described in, e.g., U.S. Pat. No. 4,708,871; Geysen et al. Proc. Natl. Acad. Sci. USA 81:3998-4002 (1984); Geysen et al. Proc. Natl. Acad. Sci. USA 82:178-182 (1985); Geysen et al. Molec. Immunol. 23:709-715 (1986 See, e.g., Epitope Mapping Protocols, supra. Additionally, antigenic regions of proteins can also be predicted and identified using standard antigenicity and hydropathy plots.

An intact antibody is generally composed of two full-length heavy chains and two full-length light chains, but in some instances may include fewer chains, such as antibodies naturally occurring in camelids that may comprise only heavy chains. Antibodies as disclosed herein may be derived solely from a single source or may be “chimeric,” that is, different portions of the antibody may be derived from two different antibodies. For example, the variable or CDR regions may be derived from a rat or murine source, while the constant region is derived from a different animal source, such as a human. The antibodies or binding fragments may be produced in hybridomas, by recombinant DNA techniques, or by enzymatic or chemical cleavage of intact antibodies. Unless otherwise indicated, the term “antibody” includes derivatives, variants, fragments, and muteins thereof, examples of which are described below (Sela-Culang et al. Front Immunol. 2013; 4:302; 2013)

The term “light chain” includes a full-length light chain and fragments thereof having sufficient variable region sequence to confer binding specificity. A full-length light chain has a molecular weight of around 25,000 Daltons and includes a variable region domain (abbreviated herein as V_{L}), and a constant region domain (abbreviated herein as CL). There are two classifications of light chains, identified as kappa (κ) and lambda (λ). The term “V_{L} fragment” means a fragment of the light chain of a monoclonal antibody that includes all or part of the light chain variable region, including CDRs. A V_{L} fragment can further include light chain constant region sequences. The variable region domain of the light chain is at the amino-terminus of the polypeptide.

The term “heavy chain” includes a full-length heavy chain and fragments thereof having sufficient variable region sequence to confer binding specificity. A full-length heavy chain has a molecular weight of around 50,000 Daltons and includes a variable region domain (abbreviated herein as V_{H}), and three constant region domains (abbreviated herein as CH1, CH2, and CH3). The term “V_{H} fragment” means a fragment of the heavy chain of a monoclonal antibody that includes all or part of the heavy chain variable region, including CDRs. A V_{H} fragment can further include heavy chain constant region sequences. The number of heavy chain constant region domains will depend on the isotype. The V_{H} domain is at the amino-terminus of the polypeptide, and the CH domains are at the carboxy-terminus, with the CH3 being closest to the —COOH end. The isotype of an antibody can be IgM, IgD, IgG, IgA, or IgE and is defined by the heavy chains present of which there are five classifications: mu (μ), delta (δ), gamma (γ), alpha (α), or epsilon (ε) chains, respectively. IgG has several subtypes, including, but not limited to, IgG1, IgG2, IgG3, and IgG4. IgM subtypes include IgM1 and IgM2. IgA subtypes include IgA1 and IgA2.

Antibodies can be whole immunoglobulins of any isotype or classification, chimeric antibodies, or hybrid antibodies with specificity to two or more antigens. They may also be fragments (e.g., F(ab′)2, Fab′, Fab, Fv, and the like), including hybrid fragments. An immunoglobulin also includes natural, synthetic, or genetically engineered proteins that act like an antibody by binding to specific antigens to form a complex. The term antibody includes genetically engineered or otherwise modified forms of immunoglobulins, such as the following:

The term “monomer” means an antibody containing only one Ig unit. Monomers are the basic functional units of antibodies. The term “dimer” means an antibody containing two Ig units attached to one another via constant domains of the antibody heavy chains (the Fc, or fragment crystallizable, region). The complex may be stabilized by a joining (J) chain protein. The term “multimer” means an antibody containing more than two Ig units attached to one another via constant domains of the antibody heavy chains (the Fc region). The complex may be stabilized by a joining (J) chain protein.

The term “bivalent antibody” means an antibody that comprises two antigen-binding sites. The two binding sites may have the same antigen specificities or they may be bi-specific, meaning the two antigen-binding sites have different antigen specificities.

Bispecific antibodies are a class of antibodies that have two paratopes with different binding sites for two or more distinct epitopes. In some embodiments, bispecific antibodies can be biparatopic, wherein a bispecific antibody may specifically recognize a different epitope from the same antigen. In some embodiments, bispecific antibodies can be constructed from a pair of different single domain antibodies termed “nanobodies”. Single domain antibodies are sourced and modified from cartilaginous fish and camelids. Nanobodies can be joined together by a linker using techniques typical to a person skilled in the art; such methods for selection and joining of nanobodies are described in PCT Publication No. WO2015044386A1, No. WO2010037838A2, and Bever et al., Anal Chem. 86:7875-7882 (2014), each of which are specifically incorporated herein by reference in their entirety.

Bispecific antibodies can be constructed as: a whole IgG, Fab′2, Fab′PEG, a diabody, or alternatively as scFv. Diabodies and scFvs can be constructed without an Fc region. using only variable domains, potentially reducing the effects of anti-idiotypic reaction. Bispecific antibodies may be produced by a variety of methods including, but not limited to, fusion of hybridomas or linking of Fab′ fragments. See, e.g., Songsivilai and Lachmann, Clin. Exp. Immunol. 79:315-321 (1990); Kostelny et al., J. Immunol. 148:1547-1553 (1992), each of which are specifically incorporated by reference in their entirety.

In certain aspects, the antigen-binding domain may be multispecific or heterospecific by multimerizing with V_{H} and V_{L} region pairs that bind a different antigen. For example, the antibody may bind to, or interact with, (a) a cell surface antigen, (b) an Fc receptor on the surface of an effector cell, or (c) at least one other component. Accordingly, aspects may include, but are not limited to, bispecific, trispecific, tetraspecific, and other multispecific antibodies or antigen-binding fragments thereof that are directed to epitopes and to other targets, such as Fc receptors on effector cells.

In some embodiments, multispecific antibodies can be used and directly linked via a short flexible polypeptide chain, using routine methods known in the art. One such example is diabodies that are bivalent, bispecific antibodies in which the V_{H} and V_{L} domains are expressed on a single polypeptide chain, and utilize a linker that is too short to allow for pairing between domains on the same chain, thereby forcing the domains to pair with complementary domains of another chain creating two antigen binding sites. The linker functionality is applicable for embodiments of triabodies, tetrabodies, and higher order antibody multimers. (see, e.g., Hollinger et al., Proc Natl. Acad. Sci. USA 90:6444-6448 (1993); Polijak et al., Structure 2:1121-1123 (1994); Todorovska et al., J. Immunol. Methods 248:47-66 (2001)).

Bispecific diabodies, as opposed to bispecific whole antibodies, may also be advantageous because they can be readily constructed and expressed in E. coli. Diabodies (and other polypeptides such as antibody fragments) of appropriate binding specificities can be readily selected using phage display (WO 94/13804) from libraries. If one arm of the diabody is kept constant, for instance, with a specificity directed against a protein, then a library can be made where the other arm is varied and an antibody of appropriate specificity selected. Bispecific whole antibodies may be made by alternative engineering methods as described in Ridgeway et al., (Protein Eng., 9:616-621, 1996) and Krah et al., (N Biotechnol. 39:167-173, 2017), each of which is hereby incorporated by reference in their entirety.

Heteroconjugate antibodies are composed of two covalently linked monoclonal antibodies with different specificities. See, e.g., U.S. Pat. No. 6,010,902, incorporated herein by reference in its entirety.

The part of the Fv fragment of an antibody molecule that binds with high specificity to the epitope of the antigen is referred to herein as the “paratope.” The paratope consists of the amino acid residues that make contact with the epitope of an antigen to facilitate antigen recognition. Each of the two Fv fragments of an antibody is composed of the two variable domains, V_{H} and V_{L}, in dimerized configuration. The primary structure of each of the variable domains includes three hypervariable loops separated by, and flanked by, Framework Regions (FR). The hypervariable loops are the regions of highest primary sequences variability among the antibody molecules from any mammal. The term hypervariable loop is sometimes used interchangeably with the term “Complementarity Determining Region (CDR).” The length of the hypervariable loops (or CDRs) varies between antibody molecules. The framework regions of all antibody molecules from a given mammal have high primary sequence similarity/consensus. The consensus of framework regions can be used by one skilled in the art to identify both the framework regions and the hypervariable loops (or CDRs) which are interspersed among the framework regions. The hypervariable loops are given identifying names which distinguish their position within the polypeptide, and on which domain they occur. CDRs in the V_{L} domain are identified as L1, L2, and L3, with L1 occurring at the most distal end and L3 occurring closest to the CL domain. The CDRs may also be given the names CDR-1, CDR-2, and CDR-3. The L3 (CDR-3) is generally the region of highest variability among all antibody molecules produced by a given organism. The CDRs are regions of the polypeptide chain arranged linearly in the primary structure, and separated from each other by Framework Regions. The amino terminal (N-terminal) end of the V_{L} chain is named FR1. The region identified as FR2 occurs between L1 and L2 hypervariable loops. FR3 occurs between L2 and L3 hypervariable loops, and the FR4 region is closest to the CL domain. This structure and nomenclature is repeated for the V_{H} chain, which includes three CDRs identified as H1, H2 and H3. The majority of amino acid residues in the variable domains, or Fv fragments (V_{H} and V_{L}), are part of the framework regions (approximately 85%). The three dimensional, or tertiary, structure of an antibody molecule is such that the framework regions are more internal to the molecule and provide the majority of the structure, with the CDRs on the external surface of the molecule.

Several methods have been developed and can be used by one skilled in the art to identify the exact amino acids that constitute each of these regions. This can be done using any of a number of multiple sequence alignment methods and algorithms, which identify the conserved amino acid residues that make up the framework regions, therefore identifying the CDRs that may vary in length but are located between framework regions. Three commonly used methods have been developed for identification of the CDRs of antibodies: Kabat (as described in T. T. Wu and E. A. Kabat, “AN ANALYSIS OF THE SEQUENCES OF THE VARIABLE REGIONS OF BENCE JONES PROTEINS AND MYELOMA LIGHT CHAINS AND THEIR IMPLICATIONS FOR ANTIBODY COMPLEMENTARITY,” J Exp Med, vol. 132, no. 2, pp. 211-250, August 1970); Chothia (as described in C. Chothia et al., “Conformations of immunoglobulin hypervariable regions,” Nature, vol. 342, no. 6252, pp. 877-883, December 1989); and IMGT (as described in M.-P. Lefranc et al., “IMGT unique numbering for immunoglobulin and T cell receptor variable domains and Ig superfamily V-like domains,” Developmental & Comparative Immunology, vol. 27, no. 1, pp. 55-77, January 2003). These methods each include unique numbering systems for the identification of the amino acid residues that constitute the variable regions. In most antibody molecules, the amino acid residues that actually contact the epitope of the antigen occur in the CDRs, although in some cases, residues within the framework regions contribute to antigen binding.

One skilled in the art can use any of several methods to determine the paratope of an antibody. These methods include: 1) Computational predictions of the tertiary structure of the antibody/epitope binding interactions based on the chemical nature of the amino acid sequence of the antibody variable region and composition of the epitope; 2) Hydrogen-deuterium exchange and mass spectroscopy; 3) Polypeptide fragmentation and peptide mapping approaches in which one generates multiple overlapping peptide fragments from the full length of the polypeptide and evaluates the binding affinity of these peptides for the epitope; 4) Antibody Phage Display Library analysis in which the antibody Fab fragment encoding genes of the mammal are expressed by bacteriophage in such a way as to be incorporated into the coat of the phage. This population of Fab expressing phage are then allowed to interact with the antigen which has been immobilized or may be expressed in by a different exogenous expression system. Non-binding Fab fragments are washed away, thereby leaving only the specific binding Fab fragments attached to the antigen. The binding Fab fragments can be readily isolated and the genes which encode them determined. This approach can also be used for smaller regions of the Fab fragment including Fv fragments or specific V_{H} and V_{L} domains as appropriate.

In certain aspects, affinity matured antibodies are enhanced with one or more modifications in one or more CDRs thereof that result in an improvement in the affinity of the antibody for a target antigen as compared to a parent antibody that does not possess those alteration(s). Certain affinity matured antibodies will have nanomolar or picomolar affinities for the target antigen. Affinity matured antibodies are produced by procedures known in the art, e.g., Marks et al., Bio/Technology 10:779 (1992) describes affinity maturation by V_{H} and V_{L} domain shuffling, random mutagenesis of CDR and/or framework residues employed in phage display is described by Rajpal et al., PNAS. 24: 8466-8471 (2005) and Thie et al., Methods Mol Biol. 525:309-22 (2009) in conjugation with computation methods as demonstrated in Tiller et al., Front. Immunol. 8:986 (2017).

Chimeric immunoglobulins are the products of fused genes derived from different species; “humanized” chimeras generally have the framework region (FR) from human immunoglobulins and one or more CDRs are from a non-human source.

In certain aspects, portions of the heavy and/or light chain are identical or homologous to corresponding sequences from another particular species or belonging to a particular antibody class or subclass, while the remainder of the chain(s) is identical or homologous to corresponding sequences in antibodies derived from another species or belonging to another antibody class or subclass, as well as fragments of such antibodies, so long as they exhibit the desired biological activity. U.S. Pat. No. 4,816,567; and Morrison et al., Proc. Natl. Acad. Sci. USA 81:6851 (1984). For methods relating to chimeric antibodies, see, e.g., U.S. Pat. No. 4,816,567; and Morrison et al., Proc. Natl. Acad. Sci. USA 81:6851-6855 (1985), each of which are specifically incorporated herein by reference in their entirety. CDR grafting is described, for example, in U.S. Pat. Nos. 6,180,370, 5,693,762, 5,693,761, 5,585,089, and 5,530,101, which are all hereby incorporated by reference for all purposes.

In some embodiments, minimizing the antibody polypeptide sequence from the non-human species optimizes chimeric antibody function and reduces immunogenicity. Specific amino acid residues from non-antigen recognizing regions of the non-human antibody are modified to be homologous to corresponding residues in a human antibody or isotype. One example is the “CDR-grafted” antibody, in which an antibody comprises one or more CDRs from a particular species or belonging to a specific antibody class or subclass, while the remainder of the antibody chain(s) is identical or homologous to a corresponding sequence in antibodies derived from another species or belonging to another antibody class or subclass. For use in humans, the V region composed of CDR1, CDR2, and partial CDR3 for both the light and heavy chain variance region from a non-human immunoglobulin, are grafted with a human antibody framework region, replacing the naturally occurring antigen receptors of the human antibody with the non-human CDRs. In some instances, corresponding non-human residues replace framework region residues of the human immunoglobulin. Furthermore, humanized antibodies may comprise residues that are not found in the recipient antibody or in the donor antibody to further refine performance. The humanized antibody may also comprise at least a portion of an immunoglobulin constant region (Fc), typically that of a human immunoglobulin. See, e.g., Jones et al., Nature 321:522 (1986); Riechmann et al., Nature 332:323 (1988); Presta, Curr. Op. Struct. Biol. 2:593 (1992); Vaswani and Hamilton, Ann. Allergy, Asthma and Immunol. 1:105 (1998); Harris, Biochem. Soc. Transactions 23; 1035 (1995); Hurle and Gross, Curr. Op. Biotech. 5:428 (1994); Verhocyen et al., Science 239:1534-36 (1988).

Intrabodies are intracellularly localized immunoglobulins that bind to intracellular antigens as opposed to secreted antibodies, which bind antigens in the extracellular space.

Polyclonal antibody preparations typically include different antibodies against different determinants (epitopes). In order to produce polyclonal antibodies, a host, such as a rabbit or goat, is immunized with the antigen or antigen fragment, generally with an adjuvant and, if necessary, coupled to a carrier. Antibodies to the antigen are subsequently collected from the sera of the host. The polyclonal antibody can be affinity purified against the antigen rendering it monospecific.

Monoclonal antibodies or “mAb” refer to an antibody obtained from a population of homogeneous antibodies from an exclusive parental cell, e.g., the population is identical except for naturally occurring mutations that may be present in minor amounts. Each monoclonal antibody is directed against a single antigenic determinant.

### A. Functional Antibody Fragments and Antigen-Binding Fragments

### 1. Antigen-Binding Fragments

Certain aspects relate to antibody fragments, such as antibody fragments that bind to and/or neutralize inflammatory mediators. The term functional antibody fragment includes antigen-binding fragments of an antibody that retain the ability to specifically bind to an antigen. These fragments are constituted of various arrangements of the variable region heavy chain (V_{H}) and/or light chain (V_{L}); and in some embodiments, include constant region heavy chain 1 (CH1) and light chain (CL). In some embodiments, they lack the Fc region constituted of heavy chain 2 (CH2) and 3 (CH3) domains. Embodiments of antigen binding fragments and the modifications thereof may include: (i) the Fab fragment type constituted with the V_{L}, V_{H}, CL, and CH1 domains; (ii) the Fd fragment type constituted with the V_{H} and CH1 domains; (iii) the Fv fragment type constituted with the V_{H} and V_{L} domains; (iv) the single domain fragment type, dAb, (Ward, 1989; McCafferty et al., 1990; Holt et al., 2003) constituted with a single V_{H} or V_{L} domain; (v) isolated complementarity determining region (CDR) regions. Such terms are described, for example, in Harlow and Lane, Antibodies: A Laboratory Manual, Cold Spring Harbor Laboratory, NY (1989); Molec. Biology and Biotechnology: A Comprehensive Desk Reference (Myers, R. A. (ed.), New York: VCH Publisher, Inc.); Huston et al., Cell Biophysics, 22:189-224 (1993); Pluckthun and Skerra, Meth. Enzymol., 178:497-515 (1989) and in Day, E. D., Advanced Immunochemistry, 2d ed., Wiley-Liss, Inc. New York, N.Y. (1990); Antibodies, 4:259-277 (2015). The citations in this paragraph are all incorporated by reference.

Antigen-binding fragments also include fragments of an antibody that retain exactly, at least, or at most 1, 2, or 3 complementarity determining regions (CDRs) from a light chain variable region. Fusions of CDR-containing sequences to an Fc region (or a CH2 or CH3 region thereof) are included within the scope of this definition including, for example, scFv fused, directly or indirectly, to an Fc region are included herein.

The term Fab fragment means a monovalent antigen-binding fragment of an antibody containing the V_{L}, V_{H}, CL and CH1 domains. The term Fab′ fragment means a monovalent antigen-binding fragment of a monoclonal antibody that is larger than a Fab fragment. For example, a Fab′ fragment includes the V_{L}, V_{H}, CL and CH1 domains and all or part of the hinge region. The term F(ab′)2 fragment means a bivalent antigen-binding fragment of a monoclonal antibody comprising two Fab′ fragments linked by a disulfide bridge at the hinge region. An F(ab′)2 fragment includes, for example, all or part of the two V_{H} and V_{L} domains, and can further include all or part of the two CL and CH1 domains.

The term Fd fragment means a fragment of the heavy chain of a monoclonal antibody, which includes all or part of the V_{H}, including the CDRs. An Fd fragment can further include CH1 region sequences.

The term Fv fragment means a monovalent antigen-binding fragment of a monoclonal antibody, including all or part of the V_{L} and V_{H}, and absent of the CL and CH1 domains. The V_{L} and V_{H} include, for example, the CDRs. Single-chain antibodies (sFv or scFv) are Fv molecules in which the V_{L} and V_{H} regions have been connected by a flexible linker to form a single polypeptide chain, which forms an antigen-binding fragment. Single chain antibodies are discussed in detail in International Patent Application Publication No. WO 88/01649 and U.S. Pat. Nos. 4,946,778 and 5,260,203, the disclosures of which are herein incorporated by reference. The term (scFv)2 means bivalent or bispecific sFv polypeptide chains that include oligomerization domains at their C-termini, separated from the sFv by a hinge region (Pack et al. 1992). The oligomerization domain comprises self-associating a-helices, e.g., leucine zippers, which can be further stabilized by additional disulfide bonds. (scFv)2 fragments are also known as “miniantibodies” or “minibodies.”

A single domain antibody is an antigen-binding fragment containing only a V_{H} or the V_{L} domain. In some instances, two or more V_{H} regions are covalently joined with a peptide linker to create a bivalent domain antibody. The two V_{H} regions of a bivalent domain antibody may target the same or different antigens.

### 2. Fragment Crystallizable Region, Fc

An Fc region contains two heavy chain fragments comprising the CH2 and CH3 domains of an antibody. The two heavy chain fragments are held together by two or more disulfide bonds and by hydrophobic interactions of the CH3 domains. The term “Fc polypeptide” as used herein includes native and mutein forms of polypeptides derived from the Fc region of an antibody. Truncated forms of such polypeptides containing the hinge region that promotes dimerization are included.

### B. Polypeptides With Antibody CDRs & Scaffolding Domains That Display the CDRs

Antigen-binding peptide scaffolds, such as complementarity-determining regions (CDRs), are used to generate protein-binding molecules in accordance with the embodiments. Generally, a person skilled in the art can determine the type of protein scaffold on which to graft at least one of the CDRs. It is known that scaffolds, optimally, must meet a number of criteria such as: good phylogenetic conservation; known three-dimensional structure; small size; few or no post-transcriptional modifications; and/or be easy to produce, express, and purify. Skerra, J Mol Recognit, 13:167-87 (2000).

The protein scaffolds can be sourced from, but not limited to: fibronectin type III FN3 domain (known as “monobodies”), fibronectin type III domain 10, lipocalin, anticalin, Z-domain of protein A of Staphylococcus aureus, thioredoxin A or proteins with a repeated motif such as the “ankyrin repeat”, the “armadillo repeat”, the “leucine-rich repeat” and the “tetratricopeptide repeat”. Such proteins are described in US Patent Publication Nos. 2010/0285564, 2006/0058510, 2006/0088908, 2005/0106660, and PCT Publication No. WO2006/056464, each of which are specifically incorporated herein by reference in their entirety. Scaffolds derived from toxins from scorpions, insects, plants, mollusks, etc., and the protein inhibiters of neuronal NO synthase (PIN) may also be used.

### C. Types of Antibodies

Antibodies can be whole immunoglobulins of any isotype or classification, chimeric antibodies, or hybrid antibodies with specificity to two or more antigens. They may also be fragments (e.g., F(ab′)2, Fab′, Fab, Fv, and the like), including hybrid fragments. An immunoglobulin also includes natural, synthetic, or genetically engineered proteins that act like an antibody by binding to specific antigens to form a complex. The term antibody includes genetically engineered or otherwise modified forms of immunoglobulins.

The term “monomer” means an antibody containing only one Ig unit. Monomers are the basic functional units of antibodies. The term “dimer” means an antibody containing two Ig units attached to one another via constant domains of the antibody heavy chains (the Fc, or fragment crystallizable, region). The complex may be stabilized by a joining (J) chain protein. The term “multimer” means an antibody containing more than two Ig units attached to one another via constant domains of the antibody heavy chains (the Fc region). The complex may be stabilized by a joining (J) chain protein.

The term “bivalent antibody” means an antibody that comprises two antigen-binding sites. The two binding sites may have the same antigen specificities or they may be bi-specific, meaning the two antigen-binding sites have different antigen specificities.

Bispecific antibodies are a class of antibodies that have two paratopes with different binding sites for two or more distinct epitopes. In some embodiments, bispecific antibodies can be biparatopic, wherein a bispecific antibody may specifically recognize a different epitope from the same antigen. In some embodiments, bispecific antibodies can be constructed from a pair of different single domain antibodies termed “nanobodies”. Single domain antibodies are sourced and modified from cartilaginous fish and camelids. Nanobodies can be joined together by a linker using techniques typical to a person skilled in the art; such methods for selection and joining of nanobodies are described in PCT Publication No. WO2015044386A1, No. WO2010037838A2, and Bever et al., Anal Chem. 86:7875-7882 (2014), each of which are specifically incorporated herein by reference in their entirety.

Bispecific antibodies can be constructed as: a whole IgG, Fab′2, Fab′PEG, a diabody, or alternatively as scFv. Diabodies and scFvs can be constructed without an Fc region, using only variable domains, potentially reducing the effects of anti-idiotypic reaction. Bispecific antibodies may be produced by a variety of methods including, but not limited to, fusion of hybridomas or linking of Fab′ fragments. See, e.g., Songsivilai and Lachmann, Clin. Exp. Immunol. 79:315-321 (1990); Kostelny et al., J. Immunol. 148:1547-1553 (1992), each of which are specifically incorporated by reference in their entirety.

In certain aspects, the antigen-binding domain may be multispecific or heterospecific by multimerizing with V_{H} and V_{L} region pairs that bind a different antigen. For example, the antibody may bind to, or interact with, (a) a cell surface antigen, (b) an Fc receptor on the surface of an effector cell, or (c) at least one other component. Accordingly, aspects may include, but are not limited to, bispecific, trispecific, tetraspecific, and other multispecific antibodies or antigen-binding fragments thereof that are directed to epitopes and to other targets, such as Fc receptors on effector cells.

In some embodiments, multispecific antibodies can be used and directly linked via a short flexible polypeptide chain, using routine methods known in the art. One such example is diabodies that are bivalent, bispecific antibodies in which the V_{H} and V_{L} domains are expressed on a single polypeptide chain, and utilize a linker that is too short to allow for pairing between domains on the same chain, thereby forcing the domains to pair with complementary domains of another chain creating two antigen binding sites. The linker functionality is applicable for embodiments of triabodies, tetrabodies, and higher order antibody multimers. (see, e.g., Hollinger et al., Proc Natl. Acad. Sci. USA 90:6444-6448 (1993); Polijak et al., Structure 2:1121-1123 (1994); Todorovska et al., J. Immunol. Methods 248:47-66 (2001)).

Bispecific diabodies, as opposed to bispecific whole antibodies, may also be advantageous because they can be readily constructed and expressed in E. coli. Diabodies (and other polypeptides such as antibody fragments) of appropriate binding specificities can be readily selected using phage display (WO94/13804) from libraries. If one arm of the diabody is kept constant, for instance, with a specificity directed against a protein, then a library can be made where the other arm is varied and an antibody of appropriate specificity selected. Bispecific whole antibodies may be made by alternative engineering methods as described in Ridgeway et al., (Protein Eng., 9:616-621, 1996) and Krah et al., (N Biotechnol. 39:167-173, 2017), each of which is hereby incorporated by reference in their entirety.

Heteroconjugate antibodies are composed of two covalently linked monoclonal antibodies with different specificities. See, e.g., U.S. Pat. No. 6,010,902, incorporated herein by reference in its entirety.

The part of the Fv fragment of an antibody molecule that binds with high specificity to the epitope of the antigen is referred to herein as the “paratope.” The paratope consists of the amino acid residues that make contact with the epitope of an antigen to facilitate antigen recognition. Each of the two Fv fragments of an antibody is composed of the two variable domains, V_{H} and V_{L}, in dimerized configuration. The primary structure of each of the variable domains includes three hypervariable loops separated by, and flanked by, Framework Regions (FR). The hypervariable loops are the regions of highest primary sequences variability among the antibody molecules from any mammal. The term hypervariable loop is sometimes used interchangeably with the term “Complementarity Determining Region (CDR).” The length of the hypervariable loops (or CDRs) varies between antibody molecules. The framework regions of all antibody molecules from a given mammal have high primary sequence similarity/consensus. The consensus of framework regions can be used by one skilled in the art to identify both the framework regions and the hypervariable loops (or CDRs) which are interspersed among the framework regions. The hypervariable loops are given identifying names which distinguish their position within the polypeptide, and on which domain they occur. CDRs in the V_{L} domain are identified as L1, L2, and L3, with L1 occurring at the most distal end and L3 occurring closest to the CL domain. The CDRs may also be given the names CDR-1, CDR-2, and CDR-3. The L3 (CDR-3) is generally the region of highest variability among all antibody molecules produced by a given organism. The CDRs are regions of the polypeptide chain arranged linearly in the primary structure, and separated from each other by Framework Regions. The amino terminal (N-terminal) end of the V_{L} chain is named FR1. The region identified as FR2 occurs between L1 and L2 hypervariable loops. FR3 occurs between L2 and L3 hypervariable loops, and the FR4 region is closest to the CL domain. This structure and nomenclature is repeated for the V_{H} chain, which includes three CDRs identified as H1, H2 and H3. The majority of amino acid residues in the variable domains, or Fv fragments (V_{H} and V_{L}), are part of the framework regions (approximately 85%). The three dimensional, or tertiary, structure of an antibody molecule is such that the framework regions are more internal to the molecule and provide the majority of the structure, with the CDRs on the external surface of the molecule.

Several methods have been developed and can be used by one skilled in the art to identify the exact amino acids that constitute each of these regions. This can be done using any of a number of multiple sequence alignment methods and algorithms, which identify the conserved amino acid residues that make up the framework regions, therefore identifying the CDRs that may vary in length but are located between framework regions. Three commonly used methods have been developed for identification of the CDRs of antibodies: Kabat (as described in T. T. Wu and E. A. Kabat, “AN ANALYSIS OF THE SEQUENCES OF THE VARIABLE REGIONS OF BENCE JONES PROTEINS AND MYELOMA LIGHT CHAINS AND THEIR IMPLICATIONS FOR ANTIBODY COMPLEMENTARITY,” J Exp Med, vol. 132, no. 2, pp. 211-250, August 1970); Chothia (as described in C. Chothia et al., “Conformations of immunoglobulin hypervariable regions,” Nature, vol. 342, no. 6252, pp. 877-883, December 1989); and IMGT (as described in M.-P. Lefranc et al., “IMGT unique numbering for immunoglobulin and T cell receptor variable domains and Ig superfamily V-like domains,” Developmental & Comparative Immunology, vol. 27, no. 1, pp. 55-77, January 2003). These methods each include unique numbering systems for the identification of the amino acid residues that constitute the variable regions. In most antibody molecules, the amino acid residues that actually contact the epitope of the antigen occur in the CDRs, although in some cases, residues within the framework regions contribute to antigen binding.

One skilled in the art can use any of several methods to determine the paratope of an antibody. These methods include:
1) Computational predictions of the tertiary structure of the antibody/epitope binding interactions based on the chemical nature of the amino acid sequence of the antibody variable region and composition of the epitope.
2) Hydrogen-deuterium exchange and mass spectroscopy
3) Polypeptide fragmentation and peptide mapping approaches in which one generates multiple overlapping peptide fragments from the full length of the polypeptide and evaluates the binding affinity of these peptides for the epitope.
4) Antibody Phage Display Library analysis in which the antibody Fab fragment encoding genes of the mammal are expressed by bacteriophage in such a way as to be incorporated into the coat of the phage. This population of Fab expressing phage are then allowed to interact with the antigen which has been immobilized or may be expressed in by a different exogenous expression system. Non-binding Fab fragments are washed away, thereby leaving only the specific binding Fab fragments attached to the antigen. The binding Fab fragments can be readily isolated and the genes which encode them determined. This approach can also be used for smaller regions of the Fab fragment including Fv fragments or specific V_{H} and V_{L} domains as appropriate.

In certain aspects, affinity matured antibodies are enhanced with one or more modifications in one or more CDRs thereof that result in an improvement in the affinity of the antibody for a target antigen as compared to a parent antibody that does not possess those alteration(s). Certain affinity matured antibodies will have nanomolar or picomolar affinities for the target antigen. Affinity matured antibodies are produced by procedures known in the art, e.g., Marks et al., Bio/Technology 10:779 (1992) describes affinity maturation by V_{H} and V_{L} domain shuffling, random mutagenesis of CDR and/or framework residues employed in phage display is described by Rajpal et al., PNAS. 24:8466-8471 (2005) and Thie et al., Methods Mol Biol. 525:309-22 (2009) in conjugation with computation methods as demonstrated in Tiller et al., Front. Immunol. 8:986 (2017).

Chimeric immunoglobulins are the products of fused genes derived from different species; “humanized” chimeras generally have the framework region (FR) from human immunoglobulins and one or more CDRs are from a non-human source.

In certain aspects, portions of the heavy and/or light chain are identical or homologous to corresponding sequences from another particular species or belonging to a particular antibody class or subclass, while the remainder of the chain(s) is identical or homologous to corresponding sequences in antibodies derived from another species or belonging to another antibody class or subclass, as well as fragments of such antibodies, so long as they exhibit the desired biological activity. U.S. Pat. No. 4,816,567; and Morrison et al., Proc. Natl. Acad. Sci. USA 81:6851 (1984). For methods relating to chimeric antibodies, see, e.g., U.S. Pat. No. 4,816,567; and Morrison et al., Proc. Natl. Acad. Sci. USA 81:6851-6855 (1985), each of which are specifically incorporated herein by reference in their entirety. CDR grafting is described, for example, in U.S. Pat. Nos. 6,180,370, 5,693,762, 5,693,761, 5,585,089, and 5,530,101, which are all hereby incorporated by reference for all purposes.

In some embodiments, minimizing the antibody polypeptide sequence from the non-human species optimizes chimeric antibody function and reduces immunogenicity. Specific amino acid residues from non-antigen recognizing regions of the non-human antibody are modified to be homologous to corresponding residues in a human antibody or isotype. One example is the “CDR-grafted” antibody, in which an antibody comprises one or more CDRs from a particular species or belonging to a specific antibody class or subclass, while the remainder of the antibody chain(s) is identical or homologous to a corresponding sequence in antibodies derived from another species or belonging to another antibody class or subclass. For use in humans, the V region composed of CDR1, CDR2, and partial CDR3 for both the light and heavy chain variance region from a non-human immunoglobulin, are grafted with a human antibody framework region, replacing the naturally occurring antigen receptors of the human antibody with the non-human CDRs. In some instances, corresponding non-human residues replace framework region residues of the human immunoglobulin. Furthermore, humanized antibodies may comprise residues that are not found in the recipient antibody or in the donor antibody to further refine performance. The humanized antibody may also comprise at least a portion of an immunoglobulin constant region (Fc), typically that of a human immunoglobulin. See, e.g., Jones et al., Nature 321:522 (1986); Riechmann et al., Nature 332:323 (1988); Presta, Curr. Op. Struct. Biol. 2:593 (1992); Vaswani and Hamilton, Ann. Allergy, Asthma and Immunol. 1:105 (1998); Harris, Biochem. Soc. Transactions 23; 1035 (1995); Hurle and Gross, Curr. Op. Biotech. 5:428 (1994); Verhocyen et al., Science 239:1534-36 (1988).

Intrabodies are intracellularly localized immunoglobulins that bind to intracellular antigens as opposed to secreted antibodies, which bind antigens in the extracellular space.

Polyclonal antibody preparations typically include different antibodies against different determinants (epitopes). In order to produce polyclonal antibodies, a host, such as a rabbit or goat, is immunized with the antigen or antigen fragment, generally with an adjuvant and, if necessary, coupled to a carrier. Antibodies to the antigen are subsequently collected from the sera of the host. The polyclonal antibody can be affinity purified against the antigen rendering it monospecific.

Monoclonal antibodies or “mAb” refer to an antibody obtained from a population of homogeneous antibodies from an exclusive parental cell, e.g., the population is identical except for naturally occurring mutations that may be present in minor amounts. Each monoclonal antibody is directed against a single antigenic determinant.

### D. Functional Antibody Fragments and Antigen-Binding Fragments

### 1. Antigen-Binding Fragments

Certain aspects relate to antibody fragments, such as antibody fragments that bind to antigen. The term functional antibody fragment includes antigen-binding fragments of an antibody that retain the ability to specifically bind to an antigen. These fragments are constituted of various arrangements of the variable region heavy chain (V_{H} ) and/or light chain (V_{L}); and in some embodiments, include constant region heavy chain 1 (CH1) and light chain (CL). In some embodiments, they lack the Fc region constituted of heavy chain 2 (CH2) and 3 (CH3) domains. Embodiments of antigen binding fragments and the modifications thereof may include: (i) the Fab fragment type constituted with the V_{L}, V_{H}, CL, and CH1 domains; (ii) the Fd fragment type constituted with the V_{H} and CH1 domains; (iii) the Fv fragment type constituted with the V_{H} and V_{L} domains; (iv) the single domain fragment type, dAb, (Ward, 1989; McCafferty et al., 1990; Holt et al., 2003) constituted with a single V_{H} or V_{L} domain; (v) isolated complementarity determining region (CDR) regions. Such terms are described, for example, in Harlow and Lane, Antibodies: A Laboratory Manual, Cold Spring Harbor Laboratory, NY (1989); Molec. Biology and Biotechnology: A Comprehensive Desk Reference (Myers, R. A. (cd.), New York: VCH Publisher, Inc.); Huston et al., Cell Biophysics, 22:189-224 (1993); Pluckthun and Skerra, Meth. Enzymol., 178:497-515 (1989) and in Day, E. D., Advanced Immunochemistry, 2d ed., Wiley-Liss, Inc. New York, N.Y. (1990); Antibodies, 4:259-277 (2015), each of which are incorporated by reference.

Antigen-binding fragments also include fragments of an antibody that retain exactly, at least, or at most 1, 2, or 3 complementarity determining regions (CDRs) from a light chain variable region. Fusions of CDR-containing sequences to an Fc region (or a CH2 or CH3 region thereof) are included within the scope of this definition including, for example, scFv fused, directly or indirectly, to an Fc region are included herein.

The term Fab fragment means a monovalent antigen-binding fragment of an antibody containing the V_{L}, V_{H}, CL and CH1 domains. The term Fab′ fragment means a monovalent antigen-binding fragment of a monoclonal antibody that is larger than a Fab fragment. For example, a Fab′ fragment includes the V_{L}, V_{H}, CL and CH1 domains and all or part of the hinge region. The term F(ab′)2 fragment means a bivalent antigen-binding fragment of a monoclonal antibody comprising two Fab′ fragments linked by a disulfide bridge at the hinge region. An F(ab′)2 fragment includes, for example, all or part of the two V_{H} and V_{L} domains, and can further include all or part of the two CL and CH1 domains.

The term Fd fragment means a fragment of the heavy chain of a monoclonal antibody, which includes all or part of the V_{H}, including the CDRs. An Fd fragment can further include CH1 region sequences.

The term Fv fragment means a monovalent antigen-binding fragment of a monoclonal antibody, including all or part of the V_{L} and V_{H}, and absent of the CL and CH1 domains. The V_{L} and V_{H} include, for example, the CDRs. Single-chain antibodies (sFv or scFv) are Fv molecules in which the V_{L} and V_{H} regions have been connected by a flexible linker to form a single polypeptide chain, which forms an antigen-binding fragment. Single chain antibodies are discussed in detail in International Patent Application Publication No. WO 88/01649 and U.S. Pat. Nos. 4,946,778 and 5,260,203, the disclosures of which are herein incorporated by reference. The term (scFv)2 means bivalent or bispecific sFv polypeptide chains that include oligomerization domains at their C-termini, separated from the sFv by a hinge region (Pack et al. 1992). The oligomerization domain comprises self-associating a-helices, e.g., leucine zippers, which can be further stabilized by additional disulfide bonds. (scFv)2 fragments are also known as “miniantibodies” or “minibodies.”

single domain antibody is an antigen-binding fragment containing only a V_{H} or the V_{L} domain. In some instances, two or more V_{H} regions are covalently joined with a peptide linker to create a bivalent domain antibody. The two V_{H} regions of a bivalent domain antibody may target the same or different antigens.

### 2. Fragment Crystallizable Region, Fc

An Fc region contains two heavy chain fragments comprising the CH2 and CH3 domains of an antibody. The two heavy chain fragments are held together by two or more disulfide bonds and by hydrophobic interactions of the CH3 domains. The term “Fc polypeptide” as used herein includes native and mutein forms of polypeptides derived from the Fc region of an antibody. Truncated forms of such polypeptides containing the hinge region that promotes dimerization are included.

### E. Antibody Binding

The term “selective binding agent” refers to a molecule that binds to an antigen. Non-limiting examples include antibodies, antigen-binding fragments, scFv, Fab, Fab′, F(ab′)2, single chain antibodies, peptides, peptide fragments and proteins.

The term “binding” refers to a direct association between two molecules, due to, for example, covalent, electrostatic, hydrophobic, and ionic and/or hydrogen-bond interactions, including interactions such as salt bridges and water bridges. “Immunologically reactive” means that the selective binding agent or antibody of interest will bind with antigens present in a biological sample. The term “immune complex” refers the combination formed when an antibody or selective binding agent binds to an epitope on an antigen.

### 1. Affinity/Avidity

The term “affinity” refers the strength with which an antibody or selective binding agent binds an epitope. In antibody binding reactions, this is expressed as the affinity constant (Ka or ka sometimes referred to as the association constant) for any given antibody or selective binding agent. Affinity is measured as a comparison of the binding strength of the antibody to its antigen relative to the binding strength of the antibody to an unrelated amino acid sequence. Affinity can be expressed as, for example, 20-fold greater binding ability of the antibody to its antigen then to an unrelated amino acid sequence. As used herein, the term “avidity” refers to the resistance of a complex of two or more agents to dissociation after dilution. The terms “immunoreactive” and “preferentially binds” are used interchangeably herein with respect to antibodies and/or selective binding agent.

There are several experimental methods that can be used by one skilled in the art to evaluate the binding affinity of any given antibody or selective binding agent for its antigen. This is generally done by measuring the equilibrium dissociation constant (KD or Kd), using the equation KD=koff/kon=[A][B]/[AB]. The term koff is the rate of dissociation between the antibody and antigen per unit time, and is related to the concentration of antibody and antigen present in solution in the unbound form at equilibrium. The term kon is the rate of antibody and antigen association per unit time, and is related to the concentration of the bound antigen-antibody complex at equilibrium. The units used for measuring the KD are mol/L (molarity, or M), or concentration. The Ka of an antibody is the opposite of the KD, and is determined by the equation Ka=1/KD. Examples of some experimental methods that can be used to determine the KD value are: enzyme-linked immunosorbent assays (ELISA), isothermal titration calorimetry (ITC), fluorescence anisotropy, surface plasmon resonance (SPR), and affinity capillary electrophoresis (ACE). The affinity constant (Ka) of an antibody is the opposite of the KD, and is determined by the equation Ka=1/KD.

Antibodies deemed useful in certain embodiments may have an affinity constant (Ka) of about, at least about, or at most about 106, 107, 108, 109, or 1010 M or any range derivable therein. Similarly, in some embodiments, antibodies may have a dissociation constant of about, at least about or at most about 10−6, 10−7, 10−8, 10−9, 10−10 M, or any range derivable therein. These values are reported for antibodies discussed herein and the same assay may be used to evaluate the binding properties of such antibodies. An antibody of the invention is said to “specifically bind” its target antigen when the dissociation constant (KD) is ≤10−8 M. The antibody specifically binds antigen with “high affinity” when the KD is ≤5×10−9 M, and with “very high affinity” when the KD is ≤5×10−10 M.

### 2. Epitope Specificity

The epitope of an antigen is the specific region of the antigen for which an antibody has binding affinity. In the case of protein or polypeptide antigens, the epitope is the specific residues (or specified amino acids or protein segment) that the antibody binds with high affinity. An antibody does not necessarily contact every residue within the protein. Nor does every single amino acid substitution or deletion within a protein necessarily affect binding affinity. For purposes of this specification and the accompanying claims, the terms “epitope” and “antigenic determinant” are used interchangeably to refer to the site on an antigen to which B and/or T cells respond or recognize. Polypeptide epitopes can be formed from both contiguous amino acids and noncontiguous amino acids juxtaposed by tertiary folding of a polypeptide. An epitope typically includes at least 3, and typically 5-10 amino acids in a unique spatial conformation.

Epitope specificity of an antibody can be determined in a variety of ways. One approach, for example, involves testing a collection of overlapping peptides of about 15 amino acids spanning the full sequence of the protein and differing in increments of a small number of amino acids (e.g., 3 to 30 amino acids). The peptides are immobilized in separate wells of a microtiter dish. Immobilization can be accomplished, for example, by biotinylating one terminus of the peptides. This process may affect the antibody affinity for the epitope, therefore different samples of the same peptide can be biotinylated at the N and C terminus and immobilized in separate wells for the purposes of comparison. This is useful for identifying end-specific antibodies. Optionally, additional peptides can be included terminating at a particular amino acid of interest. This approach is useful for identifying end-specific antibodies to internal fragments. An antibody or antigen-binding fragment is screened for binding to each of the various peptides. The epitope is defined as a segment of amino acids that is common to all peptides to which the antibody shows high affinity binding.

### 3. Modification of Antibody Antigen-Binding Domains

It is understood that the antibodies of the present disclosure may be modified, such that they are substantially identical to the antibody polypeptide sequences, or fragments thereof, and still bind the epitopes of the present disclosure. Polypeptide sequences are “substantially identical” when optimally aligned using such programs as Clustal Omega, IGBLAST, GAP or BESTFIT using default gap weights, they share at least 80% sequence identity, at least 90% sequence identity, at least 95% sequence identity, at least 96% sequence identity, at least 97% sequence identity, at least 98% sequence identity, or at least 99% sequence identity or any range therein.

As discussed herein, minor variations in the amino acid sequences of antibodies or antigen-binding regions thereof are contemplated as being encompassed by the present disclosure, providing that the variations in the amino acid sequence maintain at least 75%, more preferably at least 80%, at least 90%, at least 95%, at least 96%, at least 97%, at least 98% and most preferably at least 99% sequence identity. In particular, conservative amino acid replacements are contemplated.

Conservative replacements are those that take place within a family of amino acids that are related in their side chains. Genetically encoded amino acids are generally divided into families based on the chemical nature of the side chain; e.g., acidic (aspartate, glutamate), basic (lysine, arginine, histidine), nonpolar (alanine, valine, leucine, isoleucine, proline, phenylalanine, methionine, tryptophan), and uncharged polar (glycine, asparagine, glutamine, cysteine, serine, threonine, tyrosine). For example, it is reasonable to expect that an isolated replacement of a leucine moiety with an isoleucine or valine moiety, or a similar replacement of an amino acid with a structurally related amino acid in the same family, will not have a major effect on the binding or properties of the resulting molecule, especially if the replacement does not involve an amino acid within a framework site. Whether an amino acid change results in a functional peptide can readily be determined by assaying the specific activity of the polypeptide derivative. Standard ELISA, Surface Plasmon Resonance (SPR), or other antibody binding assays can be performed by one skilled in the art to make a quantitative comparison of antigen binging affinity between the unmodified antibody and any polypeptide derivatives with conservative substitutions generated through any of several methods available to one skilled in the art.

Fragments or analogs of antibodies or immunoglobulin molecules can be readily prepared by those skilled in the art. Preferred amino-and carboxy-termini of fragments or analogs occur near boundaries of functional domains. Structural and functional domains can be identified by comparison of the nucleotide and/or amino acid sequence data to public or proprietary sequence databases. Preferably, computerized comparison methods are used to identify sequence motifs or predicted protein conformation domains that occur in other proteins of known structure and/or function. Standard methods to identify protein sequences that fold into a known three-dimensional structure are available to those skilled in the art; Dill and McCallum., Science 338:1042-1046 (2012). Several algorithms for predicting protein structures and the gene sequences that encode these have been developed, and many of these algorithms can be found at the National Center for Biotechnology Information (on the World Wide Web at ncbi.nlm.nih.gov/guide/proteins/) and at the Bioinformatics Resource Portal (on the World Wide Web at expasy.org/proteomics). Thus, the foregoing examples demonstrate that those of skill in the art can recognize sequence motifs and structural conformations that may be used to define structural and functional domains in accordance with the invention.

Framework modifications can be made to antibodies to decrease immunogenicity, for example, by “backmutating” one or more framework residues to a corresponding germline sequence.

It is also contemplated that the antigen-binding domain may be multi-specific or multivalent by multimerizing the antigen-binding domain with V_{H} and V_{L} region pairs that bind either the same antigen (multi-valent) or a different antigen (multi-specific).

### F. Chemical Modification of Antibodies

In some aspects, also contemplated are glycosylation variants of antibodies, wherein the number and/or type of glycosylation site(s) has been altered compared to the amino acid sequences of the parent polypeptide. Glycosylation of the polypeptides can be altered, for example, by modifying one or more sites of glycosylation within the polypeptide sequence to increase the affinity of the polypeptide for antigen (U.S. Pat. Nos. 5,714.350 and 6,350,861). In certain embodiments, antibody protein variants comprise a greater or a lesser number of N-linked glycosylation sites than the native antibody. An N-linked glycosylation site is characterized by the sequence: Asn-X-Ser or Asn-X-Thr, wherein the amino acid residue designated as X may be any amino acid residue except proline. The substitution of amino acid residues to create this sequence provides a potential new site for the addition of an N-linked carbohydrate chain. Alternatively, substitutions that eliminate or alter this sequence will prevent addition of an N-linked carbohydrate chain present in the native polypeptide. For example, the glycosylation can be reduced by the deletion of an Asn or by substituting the Asn with a different amino acid. In other embodiments, one or more new N-linked glycosylation sites are created. Antibodies typically have an N-linked glycosylation site in the Fc region.

Additional antibody variants include cysteine variants, wherein one or more cysteine residues in the parent or native amino acid sequence are deleted from or substituted with another amino acid (e.g., serine). Cysteine variants are useful, inter alia, when antibodies must be refolded into a biologically active conformation. Cysteine variants may have fewer cysteine residues than the native antibody and typically have an even number to minimize interactions resulting from unpaired cysteines.

In some aspects, the polypeptides can be pegylated to increase biological half-life by reacting the polypeptide with polyethylene glycol (PEG) or a reactive ester or aldehyde derivative of PEG, under conditions in which one or more PEG groups become attached to the polypeptide. Polypeptide pegylation may be carried out by an acylation reaction or an alkylation reaction with a reactive PEG molecule (or an analogous reactive water-soluble polymer). Methods for pegylating proteins are known in the art and can be applied to the polypeptides of the present disclosure to obtain PEGylated derivatives of antibodies. See, e.g., EP 0 154 316 and EP 0 401 384. In some aspects, the antibody is conjugated or otherwise linked to transthyretin (TTR) or a TTR variant. The TTR or TTR variant can be chemically modified with, for example, a chemical selected from the group consisting of dextran, poly (n-vinyl pyrrolidone), polyethylene glycols, propropylene glycol homopolymers, polypropylene oxide/ethylene oxide co-polymers, polyoxyethylated polyols, and polyvinyl alcohols. As used herein, the term “polyethylene glycol” is intended to encompass any of the forms of PEG that have been used to derivatize other proteins.

### 1. Conjugation

Derivatives of the antibodies and antigen binding fragments that are described herein are also provided. The derivatized antibody or fragment thereof may comprise any molecule or substance that imparts a desired property to the antibody or fragment. The derivatized antibody can comprise, for example, a detectable (or labeling) moiety (e.g., a radioactive, colorimetric, antigenic, or enzymatic molecule, or a detectable bead), a molecule that binds to another molecule (e.g., biotin or streptavidin), a therapeutic or diagnostic moiety (e.g., a radioactive, cytotoxic, or pharmaceutically active moiety), or a molecule that increases the suitability of the antibody for a particular use (e.g., administration to a subject, such as a human subject, or other in vivo or in vitro uses).

Optionally, an antibody or an immunological portion of an antibody can be chemically conjugated to, or expressed as, a fusion protein with other proteins. In some aspects, polypeptides may be chemically modified by conjugating or fusing the polypeptide to serum protein, such as human serum albumin, to increase half-life of the resulting molecule. See, e.g., EP 0322094 and EP 0 486 525. In some aspects, the polypeptides may be conjugated to a diagnostic agent and used diagnostically, for example, to monitor the development or progression of a disease and determine the efficacy of a given treatment regimen. In some aspects, the polypeptides may also be conjugated to a therapeutic agent to provide a therapy in combination with the therapeutic effect of the polypeptide. Additional suitable conjugated molecules include ribonuclease (RNase), DNase I, an antisense nucleic acid, an inhibitory RNA molecule such as a siRNA molecule, an immunostimulatory nucleic acid, aptamers, ribozymes, triplex forming molecules, and external guide sequences. The functional nucleic acid molecules may act as effectors, inhibitors, modulators, and stimulators of a specific activity possessed by a target molecule, or the functional nucleic acid molecules may possess a de novo activity independent of any other molecules.

In some aspects, disclosed are antibodies and antibody-like molecules that are linked to at least one agent to form an antibody conjugate or payload. In order to increase the efficacy of antibody molecules as diagnostic or therapeutic agents, it is conventional to link or covalently bind or complex at least one desired molecule or moiety. Such a molecule or moiety may be, but is not limited to, at least one effector or reporter molecule. Effector molecules comprise molecules having a desired activity, e.g., cytotoxic activity. Non-limiting examples of effector molecules include toxins, therapeutic enzymes, antibiotics, radiolabeled nucleotides and the like. By contrast, a reporter molecule is defined as any moiety that may be detected using an assay. Non-limiting examples of reporter molecules that have been conjugated to antibodies include enzymes, radiolabels, haptens, fluorescent labels, phosphorescent molecules, chemiluminescent molecules, chromophores, luminescent molecules, photoaffinity molecules, colored particles, or ligands.

### 2. Conjugate Types

Certain examples of antibody conjugates are those conjugates in which the antibody is linked to a detectable label. “Detectable labels” are compounds and/or elements that can be detected due to their specific functional properties, and/or chemical characteristics, the use of which allows the antibody to be detected, and/or further quantified if desired. Examples of detectable labels include, but not limited to, radioactive isotopes, fluorescers, semiconductor nanocrystals, chemiluminescers, chromophores, enzymes, enzyme substrates, enzyme cofactors, enzyme inhibitors, dyes, metal ions, metal sols, ligands (e.g., biotin, streptavidin or haptens) and the like. Particular examples of labels are, but not limited to, horseradish peroxidase (HRP), fluorescein, FITC, rhodamine, dansyl, umbelliferone, dimethyl acridinium ester (DMAE), Texas red, luminol, NADPH and α- or β-galactosidase. Antibody conjugates include those intended primarily for use in vitro, where the antibody is linked to a secondary binding ligand and/or to an enzyme to generate a colored product upon contact with a chromogenic substrate. Examples of suitable enzymes include, but are not limited to, urease, alkaline phosphatase, (horseradish) hydrogen peroxidase, or glucose oxidase. Preferred secondary binding ligands are biotin and/or avidin and streptavidin compounds. The uses of such labels is well known to those of skill in the art and are described, for example, in U.S. Pat. Nos. 3,817,837; 3,850,752; 3,939,350; 3,996,345; 4,277,437; 4,275,149 and 4,366,241; each incorporated herein by reference. Molecules containing azido groups may also be used to form covalent bonds to proteins through reactive nitrene intermediates that are generated by low intensity ultraviolet light (Potter & Haley, 1983).

In some aspects, contemplated are immunoconjugates comprising an antibody or antigen-binding fragment thereof conjugated to a cytotoxic agent such as a chemotherapeutic agent, a drug, a growth inhibitory agent, a toxin (e.g., an enzymatically active toxin of bacterial, fungal, plant, or animal origin, or fragments thereof), or a radioactive isotope (i.e., a radioconjugate). In this way, the agent of interest can be targeted directly to cells bearing cell surface antigen. The antibody and agent may be associated through non-covalent interactions such as through electrostatic forces, or by covalent bonds. Various linkers, known in the art, can be employed in order to form the immunoconjugate. Additionally, the immunoconjugate can be provided in the form of a fusion protein. In one aspect, an antibody may be conjugated to various therapeutic substances in order to target the cell surface antigen. Examples of conjugated agents include, but are not limited to, metal chelate complexes, drugs, toxins and other effector molecules, such as cytokines, lymphokines, chemokines, immunomodulators, radiosensitizers, asparaginase, carboranes, and radioactive halogens.

In antibody drug conjugates (ADC), an antibody (Ab) is conjugated to one or more drug moieties (D) through a linker (L). The ADC may be prepared by several routes, employing organic chemistry reactions, conditions, and reagents known to those skilled in the art, including: (1) reaction of a nucleophilic group of an antibody with a bivalent linker reagent, to form Ab-L, via a covalent bond, followed by reaction with a drug moiety D; and (2) reaction of a nucleophilic group of a drug moiety with a bivalent linker reagent, to form D-L, via a covalent bond, followed by reaction with the nucleophilic group of an antibody. Antibody drug conjugates may also be produced by modification of the antibody to introduce electrophilic moieties, which can react with nucleophilic substituents on the linker reagent or drug. Alternatively, a fusion protein comprising the antibody and cytotoxic agent may be made, e.g., by recombinant techniques or peptide synthesis. The length of DNA may comprise respective regions encoding the two portions of the conjugate either adjacent one another or separated by a region encoding a linker peptide which does not destroy the desired properties of the conjugate. In yet another aspect, the antibody may be conjugated to a “receptor” (such as streptavidin) for utilization in tumor or cancer cell pre-targeting wherein the antibody-receptor conjugate is administered to the patient, followed by removal of unbound conjugate from the circulation using a clearing agent and then administration of a “ligand” (e.g., avidin) which is conjugated to a cytotoxic agent (e.g., a radionucleotide).

Examples of an antibody-drug conjugates known to a person skilled in the art are pro-drugs useful for the local delivery of cytotoxic or cytostatic agents, i.e. drugs to kill or inhibit tumor cells in the treatment of cancer (Syrigos and Epenetos, Anticancer Res. 19:605-614 (1999); Niculescu-Duvaz and Springer, Adv. Drg. Del. Rev. 26:151-172 (1997); U.S. Pat. No. 4,975,278). In contrast, systematic administration of these unconjugated drug agents may result in unacceptable levels of toxicity to normal cells as well as the target tumor cells (Baldwin et al., Lancet 1:603-5 (1986); Thorpe, (1985) “Antibody Carriers of Cytotoxic Agents in Cancer Therapy: A Review,” In: Monoclonal Antibodies '84: Biological and Clinical Applications, A. Pincera et al., (eds.) pp. 475-506). Both polyclonal antibodies and monoclonal antibodies have been reported as useful in these strategies (Rowland et al., Cancer Immunol. Immunother. 21:183-87 (1986)).

In certain aspects, ADC include covalent or aggregative conjugates of antibodies, or antigen-binding fragments thereof, with other proteins or polypeptides, such as by expression of recombinant fusion proteins comprising heterologous polypeptides fused to the N-terminus or C-terminus of an antibody polypeptide. For example, the conjugated peptide may be a heterologous signal (or leader) polypeptide, e.g., the yeast alpha-factor leader, or a peptide such as an epitope tag (e.g., V5-His). Antibody-containing fusion proteins may comprise peptides added to facilitate purification or identification of the antibody (e.g., poly-His). An antibody polypeptide also can be linked to the FLAG® (Sigma-Aldrich, St. Louis, Mo.) peptide as described in Hopp et al., Bio/Technology 6:1204 (1988), and U.S. Pat. No. 5,011,912. Oligomers that contain one or more antibody polypeptides may be employed as antagonists. Oligomers may be in the form of covalently linked or non-covalently linked dimers, trimers, or higher oligomers. Oligomers comprising two or more antibody polypeptides are contemplated for use. Other oligomers include heterodimers, homotrimers, heterotrimers, homotetramers, heterotetramers, etc. In certain aspects, oligomers comprise multiple antibody polypeptides joined via covalent or non-covalent interactions between peptide moieties fused to the antibody polypeptides. Such peptides may be peptide linkers (spacers), or peptides that have the property of promoting oligomerization. Leucine zippers and certain polypeptides derived from antibodies are among the peptides that can promote oligomerization of antibody polypeptides attached thereto, as described in more detail below.

### 3. Conjugation Methodology

Several methods are known in the art for the attachment or conjugation of an antibody to its conjugate moiety. Some attachment methods involve the use of a metal chelate complex employing, for example, an organic chelating agent such a diethylenetriaminepentaacetic acid anhydride (DTPA); ethylenetriaminetetraacetic acid; N-chloro-p-toluenesulfonamide; and/or tetrachloro-3-6-diphenylglycouril-3 attached to the antibody (U.S. Pat. Nos. 4,472,509 and 4,938,948, each incorporated herein by reference). Monoclonal antibodies may also be reacted with an enzyme in the presence of a coupling agent such as glutaraldehyde or periodate. Conjugates may also be made using a variety of bifunctional protein-coupling agents such as N-succinimidyl-3-(2-pyridyldithiol) propionate (SPDP), iminothiolane (IT), bifunctional derivatives of imidoesters (such as dimethyl adipimidate HCl), active esters (such as disuccinimidyl suberate), aldehydes (such as glutaraldehyde), bis-azido compounds (such as bis(p-azidobenzoyl)hexanediamine), bis-diazonium derivatives (such as bos(p-diazoniumbenzoyl)-ethylenediamine), diisocyanates (such as toluene 2,6-diisocyanate), and bis-active fluorine compounds (such as 1,5-difluoro-2,4-dinitrobenzene). In some aspects, derivatization of immunoglobulins by selectively introducing sulfhydryl groups in the Fc region of an immunoglobulin, using reaction conditions that do not alter the antibody combining site, are contemplated. Antibody conjugates produced according to this methodology are disclosed to exhibit improved longevity, specificity, and sensitivity (U.S. Pat. No. 5,196,066, incorporated herein by reference). Site-specific attachment of effector or reporter molecules, wherein the reporter or effector molecule is conjugated to a carbohydrate residue in the Fc region has also been disclosed in the literature (O'Shannessy et al., 1987).

### IV. Proteins

The present disclosure encompasses antibody proteins or polypeptides of any kind and chimeric protein or polypeptide molecules of any kind, including chimeric protein or polypeptide molecules that encompass a functional part or all of the antibody proteins or polypeptides. As used herein, a “protein” or “polypeptide” refers to a molecule comprising at least five amino acid residues. As used herein, the term “wild-type” refers to the endogenous version of a molecule that occurs naturally in an organism. In some embodiments, wild-type versions of a protein or polypeptide are employed, however, in many embodiments of the disclosure, a modified protein or polypeptide is employed to generate an immune response. The terms described above may be used interchangeably. A “modified protein” or “modified polypeptide” or a “variant” refers to a protein or polypeptide whose chemical structure, particularly its amino acid sequence, is altered with respect to the wild-type protein or polypeptide. In some embodiments, a modified/variant protein or polypeptide has at least one modified activity or function (recognizing that proteins or polypeptides may have multiple activities or functions). It is specifically contemplated that a modified/variant protein or polypeptide may be altered with respect to one activity or function yet retain a wild-type activity or function in other respects, such as immunogenicity.

Where a protein is specifically mentioned herein, it is in general a reference to a native (wild-type) or recombinant (modified) protein or, optionally, a protein in which any signal sequence has been removed. The protein may be isolated directly from the organism of which it is native, produced by recombinant DNA/exogenous expression methods, or produced by solid-phase peptide synthesis (SPPS) or other in vitro methods. In particular embodiments. there are isolated nucleic acid segments and recombinant vectors incorporating nucleic acid sequences that encode a polypeptide (e.g., an antibody or fragment thereof). The term “recombinant” may be used in conjunction with a polypeptide or the name of a specific polypeptide, and this generally refers to a polypeptide produced from a nucleic acid molecule that has been manipulated in vitro or that is a replication product of such a molecule.

In certain embodiments the size of a protein or polypeptide (wild-type or modified) may comprise, but is not limited to, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 275, 300, 325, 350, 375, 400, or 425 amino acid residues or greater, and any range derivable therein, or derivative of a corresponding amino sequence described or referenced herein. It is contemplated that polypeptides may be mutated by truncation, rendering them shorter than their corresponding wild-type form, also, they might be altered by fusing or conjugating a heterologous protein or polypeptide sequence with a particular function (e.g., for targeting or localization, for enhanced immunogenicity, for purification purposes, etc.). As used herein, the term “domain” refers to any distinct functional or structural unit of a protein or polypeptide, and generally refers to a sequence of amino acids with a structure or function recognizable by one skilled in the art.

The polypeptides, proteins, or polynucleotides encoding such polypeptides or proteins of the disclosure may include 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 (or any derivable range therein) or more variant amino acids or nucleic acid substitutions or be at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%. 85%, 86%, 87%, 88%, 89%, 90%, 91%, 92%, 93%, 94%, 95%, 96%, 97%, 98%, 99%, or 100% (or any derivable range therein) similar, identical, or homologous with at least, or at most 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 300, 400, or more contiguous amino acids or nucleic acids respectively, or any range derivable therein, of SEQ ID NOs: 4-19.

In some embodiments, the protein or polypeptide may comprise amino acids 1 to 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, or 425 or more (or any derivable range therein) of SEQ ID NOs: 5, 7, 9, 11, 13, 15, 17, or 19.

In some embodiments, the protein, polypeptide, or nucleic acid may comprise 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, or more (or any derivable range therein) contiguous amino acids of SEQ ID NOs: 4-19.

In some embodiments, the polypeptide, protein, or nucleic acid may comprise at least, at most, or exactly 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211. 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, or more (or any derivable range therein) contiguous amino acids of SEQ ID NOs: 4-19 that are at least, at most, or exactly 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% (or any derivable range therein) similar, identical, or homologous with one of SEQ ID NOS: 4-19.

In some aspects there is a nucleic acid molecule or polypeptide starting at position 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, or more (or any derivable range therein) contiguous amino acids or nucleotides of any of SEQ ID NOS: 4-19.

The nucleotide as well as the protein, polypeptide, and peptide sequences for various genes have been previously disclosed, and may be found in the recognized computerized databases. Two commonly used databases are the National Center for Biotechnology Information's Genbank® and GenPept® databases (on the World Wide Web at ncbi.nlm.nih.gov/) and The Universal Protein Resource (UniProt; on the World Wide Web at uniprot.org). The coding regions for these genes may be amplified and/or expressed using the techniques disclosed herein or as would be known to those of ordinary skill in the art.

It is contemplated that in compositions of the disclosure, there is between about 0.001 mg and about 10 mg of total polypeptide, peptide, and/or protein per ml. The concentration of protein in a composition can be about, at least about or at most about 0.001, 0.010, 0.050, 0.1, 0.2, 0.3, 0.4, 0.5, 0.6, 0.7, 0.8, 0.9, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10.0 mg/ml or more (or any range derivable therein).

### A. Variant Polypeptides

The following is a discussion of changing the amino acid subunits of an antibody- and/or receptor-related protein to create an equivalent, or even improved, second-generation variant polypeptide or peptide. For example, certain amino acids may be substituted for other amino acids in a protein or polypeptide sequence with or without appreciable loss of interactive binding capacity with structures such as, for example, antigen-binding regions of antibodies or binding sites on substrate molecules. Since it is the interactive capacity and nature of a protein that defines that protein's functional activity, certain amino acid substitutions can be made in a protein sequence and in its corresponding DNA coding sequence, and nevertheless produce a protein with similar or desirable properties. It is thus contemplated by the inventors that various changes may be made in the DNA sequences of genes which encode proteins without appreciable loss of their biological utility or activity.

The term “functionally equivalent codon” is used herein to refer to codons that encode the same amino acid, such as the six different codons for arginine. Also considered are “neutral substitutions” or “neutral mutations” which refers to a change in the codon or codons that encode biologically equivalent amino acids.

Amino acid sequence variants of the disclosure can be substitutional, insertional, or deletion variants. A variation in a polypeptide of the disclosure may affect 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or more non-contiguous or contiguous amino acids of the protein or polypeptide, as compared to wild-type. A variant can comprise an amino acid sequence that is at least 50%, 60%, 70%, 80%, or 90%, including all values and ranges there between, identical to any sequence provided or referenced herein. A variant can include 2, 3, 4, 5, 6, 7, 8, 9, 10, 11, 12, 13, 14, 15, 16, 17, 18, 19, 20, or more substitute amino acids.

It also will be understood that amino acid and nucleic acid sequences may include additional residues, such as additional N- or C-terminal amino acids, or 5′ or 3′ sequences, respectively, and yet still be essentially identical as set forth in one of the sequences disclosed herein, so long as the sequence meets the criteria set forth above, including the maintenance of biological protein activity where protein expression is concerned. The addition of terminal sequences particularly applies to nucleic acid sequences that may, for example, include various non-coding sequences flanking either of the 5′ or 3′ portions of the coding region.

Deletion variants typically lack one or more residues of the native or wild type protein. Individual residues can be deleted or a number of contiguous amino acids can be deleted. A stop codon may be introduced (by substitution or insertion) into an encoding nucleic acid sequence to generate a truncated protein.

Insertional mutants typically involve the addition of amino acid residues at a non-terminal point in the polypeptide. This may include the insertion of one or more amino acid residues. Terminal additions may also be generated and can include fusion proteins which are multimers or concatemers of one or more peptides or polypeptides described or referenced herein.

Substitutional variants typically contain the exchange of one amino acid for another at one or more sites within the protein or polypeptide, and may be designed to modulate one or more properties of the polypeptide, with or without the loss of other functions or properties. Substitutions may be conservative, that is, one amino acid is replaced with one of similar chemical properties. “Conservative amino acid substitutions” may involve exchange of a member of one amino acid class with another member of the same class. Conservative substitutions are well known in the art and include, for example, the changes of: alanine to serine; arginine to lysine; asparagine to glutamine or histidine; aspartate to glutamate; cysteine to serine; glutamine to asparagine; glutamate to aspartate; glycine to proline; histidine to asparagine or glutamine; isoleucine to leucine or valine; leucine to valine or isoleucine; lysine to arginine; methionine to leucine or isoleucine; phenylalanine to tyrosine, leucine or methionine; serine to threonine; threonine to serine; tryptophan to tyrosine; tyrosine to tryptophan or phenylalanine; and valine to isoleucine or leucine. Conservative amino acid substitutions may encompass non-naturally occurring amino acid residues, which are typically incorporated by chemical peptide synthesis rather than by synthesis in biological systems. These include peptidomimetics or other reversed or inverted forms of amino acid moieties.

Alternatively, substitutions may be “non-conservative”, such that a function or activity of the polypeptide is affected. Non-conservative changes typically involve substituting an amino acid residue with one that is chemically dissimilar, such as a polar or charged amino acid for a nonpolar or uncharged amino acid, and vice versa. Non-conservative substitutions may involve the exchange of a member of one of the amino acid classes for a member from another class.

### B. Considerations for Substitutions

One skilled in the art can determine suitable variants of polypeptides as set forth herein using well-known techniques. One skilled in the art may identify suitable areas of the molecule that may be changed without destroying activity by targeting regions not believed to be important for activity. The skilled artisan will also be able to identify amino acid residues and portions of the molecules that are conserved among similar proteins or polypeptides. In further embodiments, areas that may be important for biological activity or for structure may be subject to conservative amino acid substitutions without significantly altering the biological activity or without adversely affecting the protein or polypeptide structure.

In making such changes, the hydropathy index of amino acids may be considered. The hydropathy profile of a protein is calculated by assigning each amino acid a numerical value (“hydropathy index”) and then repetitively averaging these values along the peptide chain. Each amino acid has been assigned a value based on its hydrophobicity and charge characteristics. They are: isoleucine (+4.5); valine (+4.2); leucine (+3.8); phenylalanine (+2.8); cysteine/cysteine (+2.5); methionine (+1.9); alanine (+1.8); glycine (−0.4); threonine (−0.7); serine (−0.8); tryptophan (−0.9); tyrosine (−1.3); proline (1.6); histidine (−3.2); glutamate (−3.5); glutamine (−3.5); aspartate (−3.5); asparagine (−3.5); lysine (−3.9); and arginine (−4.5). The importance of the hydropathy amino acid index in conferring interactive biologic function on a protein is generally understood in the art (Kyte et al., J. Mol. Biol. 157:105-131 (1982)). It is accepted that the relative hydropathic character of the amino acid contributes to the secondary structure of the resultant protein or polypeptide, which in turn defines the interaction of the protein or polypeptide with other molecules, for example, enzymes, substrates, receptors, DNA, antibodies, antigens, and others. It is also known that certain amino acids may be substituted for other amino acids having a similar hydropathy index or score, and still retain a similar biological activity. In making changes based upon the hydropathy index, in certain embodiments, the substitution of amino acids whose hydropathy indices are within ±2 is included. In some aspects of the present disclosure, those that are within ±1 are included, and in other aspects of the present disclosure, those within ±0.5 are included.

It also is understood in the art that the substitution of like amino acids can be effectively made based on hydrophilicity. U.S. Pat. No. 4,554,101, incorporated herein by reference, states that the greatest local average hydrophilicity of a protein, as governed by the hydrophilicity of its adjacent amino acids, correlates with a biological property of the protein. In certain embodiments, the greatest local average hydrophilicity of a protein, as governed by the hydrophilicity of its adjacent amino acids, correlates with its immunogenicity and antigen binding, that is, as a biological property of the protein. The following hydrophilicity values have been assigned to these amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); and tryptophan (−3.4). In making changes based upon similar hydrophilicity values, in certain embodiments, the substitution of amino acids whose hydrophilicity values are within ±2 are included, in other embodiments, those which are within ±1 are included, and in still other embodiments, those within ±0.5 are included. In some instances, one may also identify epitopes from primary amino acid sequences based on hydrophilicity. These regions are also referred to as “epitopic core regions.” It is understood that an amino acid can be substituted for another having a similar hydrophilicity value and still produce a biologically equivalent and immunologically equivalent protein.

Additionally, one skilled in the art can review structure-function studies identifying residues in similar polypeptides or proteins that are important for activity or structure. In view of such a comparison, one can predict the importance of amino acid residues in a protein that correspond to amino acid residues important for activity or structure in similar proteins. One skilled in the art may opt for chemically similar amino acid substitutions for such predicted important amino acid residues.

One skilled in the art can also analyze the three-dimensional structure and amino acid sequence in relation to that structure in similar proteins or polypeptides. In view of such information, one skilled in the art may predict the alignment of amino acid residues of an antibody with respect to its three-dimensional structure. One skilled in the art may choose not to make changes to amino acid residues predicted to be on the surface of the protein, since such residues may be involved in important interactions with other molecules. Moreover, one skilled in the art may generate test variants containing a single amino acid substitution at each desired amino acid residue. These variants can then be screened using standard assays for binding and/or activity, thus yielding information gathered from such routine experiments, which may allow one skilled in the art to determine the amino acid positions where further substitutions should be avoided either alone or in combination with other mutations. Various tools available to determine secondary structure can be found on the world wide web at expasy.org/proteomics/protein_structure.

In some embodiments of the disclosure, amino acid substitutions are made that: (1) reduce susceptibility to proteolysis, (2) reduce susceptibility to oxidation, (3) alter binding affinity for forming protein complexes, (4) alter ligand or antigen binding affinities, and/or (5) confer or modify other physicochemical or functional properties on such polypeptides. For example, single or multiple amino acid substitutions (in certain embodiments, conservative amino acid substitutions) may be made in the naturally occurring sequence. Substitutions can be made in that portion of the antibody that lies outside the domain(s) forming intermolecular contacts. In such embodiments, conservative amino acid substitutions can be used that do not substantially change the structural characteristics of the protein or polypeptide (e.g., one or more replacement amino acids that do not disrupt the secondary structure that characterizes the native antibody).

### V. Nucleic Acids

In various embodiments, the disclosure encompasses nucleic acids that encode an antibody, a chimeric receptor (including a chimeric TCR or STAR) and/or encode a bi-specific T-cell engager (BiTE).

In certain embodiments, nucleic acid sequences can exist in a variety of instances such as: isolated segments and recombinant vectors of incorporated sequences or recombinant polynucleotides encoding one or both chains of an antibody, or a fragment, derivative, mutein, or variant thereof, polynucleotides sufficient for use as hybridization probes, PCR primers or sequencing primers for identifying, analyzing, mutating or amplifying a polynucleotide encoding a polypeptide, anti-sense nucleic acids for inhibiting expression of a polynucleotide, and complementary sequences of the foregoing described herein. Nucleic acids that encode the epitope to which certain of the antibodies provided herein are also provided. Nucleic acids encoding fusion proteins that include these peptides are also provided. The nucleic acids can be single-stranded or double-stranded and can comprise RNA and/or DNA nucleotides and artificial variants thereof (e.g., peptide nucleic acids).

The term “polynucleotide” refers to a nucleic acid molecule that either is recombinant or has been isolated from total genomic nucleic acid. Included within the term “polynucleotide” are oligonucleotides (nucleic acids 100 residues or less in length), recombinant vectors, including, for example, plasmids, cosmids, phage, viruses, and the like. Polynucleotides include, in certain aspects, regulatory sequences, isolated substantially away from their naturally occurring genes or protein encoding sequences. Polynucleotides may be single-stranded (coding or antisense) or double-stranded, and may be RNA, DNA (genomic, cDNA or synthetic), analogs thereof, or a combination thereof. Additional coding or non-coding sequences may, but need not, be present within a polynucleotide.

In this respect, the term “gene,” “polynucleotide,” or “nucleic acid” is used to refer to a nucleic acid that encodes a protein, polypeptide, or peptide (including any sequences required for proper transcription, post-translational modification, or localization). As will be understood by those in the art, this term encompasses genomic sequences, expression cassettes, cDNA sequences, and smaller engineered nucleic acid segments that express, or may be adapted to express, proteins, polypeptides, domains, peptides, fusion proteins, and mutants. A nucleic acid encoding all or part of a polypeptide may contain a contiguous nucleic acid sequence encoding all or a portion of such a polypeptide. It also is contemplated that a particular polypeptide may be encoded by nucleic acids containing variations having slightly different nucleic acid sequences but, nonetheless, encode the same or substantially similar protein.

In certain embodiments, there are polynucleotide variants having substantial identity to the sequences disclosed herein; those comprising at least 70%, 75%, 80%, 85%, 90%, 95%, 96%, 97%, 98%, or 99% or higher sequence identity, including all values and ranges there between, compared to a polynucleotide sequence provided herein using the methods described herein (e.g., BLAST analysis using standard parameters). In certain aspects, the isolated polynucleotide will comprise a nucleotide sequence encoding a polypeptide that has at least 90%, preferably 95% and above, identity to an amino acid sequence described herein, over the entire length of the sequence; or a nucleotide sequence complementary to said isolated polynucleotide.

The nucleic acid segments, regardless of the length of the coding sequence itself, may be combined with other nucleic acid sequences, such as promoters, polyadenylation signals, additional restriction enzyme sites, multiple cloning sites, other coding segments, and the like, such that their overall length may vary considerably. The nucleic acids can be any length. They can be, for example, 5, 10, 15, 20, 25, 30, 35, 40, 45, 50, 75, 100, 125, 175, 200, 250, 300, 350, 400, 450, 500, 750, 1000, 1500, 3000, 5000 or more nucleotides in length, and/or can comprise one or more additional sequences, for example, regulatory sequences, and/or be a part of a larger nucleic acid, for example, a vector. It is therefore contemplated that a nucleic acid fragment of almost any length may be employed, with the total length preferably being limited by the case of preparation and use in the intended recombinant nucleic acid protocol. In some cases, a nucleic acid sequence may encode a polypeptide sequence with additional heterologous coding sequences, for example to allow for purification of the polypeptide, transport, secretion, post-translational modification, or for therapeutic benefits such as targeting or efficacy. As discussed above, a tag or other heterologous polypeptide may be added to the modified polypeptide-encoding sequence, wherein “heterologous” refers to a polypeptide that is not the same as the modified polypeptide.

### VI. Antibody Production

The present disclosure includes antibodies produced for binding TdT, including TdT peptides.

### A. Antibody Production

Methods for preparing and characterizing antibodies for use in diagnostic and detection assays, for purification, and for use as therapeutics are well known in the art as disclosed in, for example, U.S. Pat. Nos. 4,011,308; 4,722,890; 4,016,043; 3,876,504; 3,770,380; and 4,372,745 (see, e.g., Antibodies: A Laboratory Manual, Cold Spring Harbor Laboratory, 1988; incorporated herein by reference). These antibodies may be polyclonal or monoclonal antibody preparations, monospecific antisera, human antibodies, hybrid or chimeric antibodies, such as humanized antibodies, altered antibodies, F(ab′)2 fragments, Fab fragments, Fv fragments, single-domain antibodies, dimeric or trimeric antibody fragment constructs, minibodies, or functional fragments thereof which bind to the antigen in question. In certain aspects, polypeptides, peptides, and proteins and immunogenic fragments thereof for use in various embodiments can also be synthesized in solution or on a solid support in accordance with conventional techniques. See, for example, Stewart and Young, (1984); Tarn et al. (1983); Merrifield, (1986); and Barany and Merrifield (1979), each incorporated herein by reference.

Briefly, a polyclonal antibody is prepared by immunizing an animal with an antigen or a portion thereof and collecting antisera from that immunized animal. The antigen may be altered compared to an antigen sequence found in nature. In some embodiments, a variant or altered antigenic peptide or polypeptide is employed to generate antibodies. Inocula are typically prepared by dispersing the antigenic composition in a physiologically tolerable diluent to form an aqueous composition. Antisera is subsequently collected by methods known in the arts, and the serum may be used as-is for various applications or else the desired antibody fraction may be purified by well-known methods, such as affinity chromatography (Harlow and Lane, Antibodies: A Laboratory Manual 1988).

Methods of making monoclonal antibodies are also well known in the art (Kohler and Milstein, 1975; Harlow and Lane, 1988, U.S. Pat. No. 4,196,265, herein incorporated by reference in its entirety for all purposes). Typically, this technique involves immunizing a suitable animal with a selected immunogenic composition, e.g., a purified or partially purified protein, polypeptide, peptide or domain. Resulting antibody-producing B-cells from the immunized animal, or all dissociated splenocytes, are then induced to fuse with cells from an immortalized cell line to form hybridomas. Myeloma cell lines suited for use in hybridoma-producing fusion procedures preferably are non-antibody-producing and have high fusion efficiency and enzyme deficiencies that render then incapable of growing in certain selective media that support the growth of only the desired fused cells (hybridomas). Typically, the fusion partner includes a property that allows selection of the resulting hybridomas using specific media. For example, fusion partners can be hypoxanthine/aminopterin/thymidine (HAT)-sensitive. Methods for generating hybrids of antibody-producing spleen or lymph node cells and myeloma cells usually comprise mixing somatic cells with myeloma cells in the presence of an agent or agents (chemical or electrical) that promote the fusion of cell membranes. Next, selection of hybridomas can be performed by culturing the cells by single-clone dilution in microtiter plates, followed by testing the individual clonal supernatants (after about two to three weeks) for the desired reactivity. Fusion procedures for making hybridomas, immunization protocols, and techniques for isolation of immunized splenocytes for fusion are known in the art.

Other techniques for producing monoclonal antibodies include the viral or oncogenic transformation of B-lymphocytes, a molecular cloning approach may be used to generate a nucleic acid or polypeptide, the selected lymphocyte antibody method (SLAM) (see, e.g., Babcook et al., Proc. Natl. Acad. Sci. USA 93:7843-7848 (1996), the preparation of combinatorial immunoglobulin phagemid libraries from RNA isolated from the spleen of the immunized animal and selection of phagemids expressing appropriate antibodies, or producing a cell expressing an antibody from a genomic sequence of the cell comprising a modified immunoglobulin locus using Cre-mediated site-specific recombination (sec, e.g., U.S. Pat. No. 6,091,001).

Monoclonal antibodies may be further purified using filtration, centrifugation, and various chromatographic methods such as HPLC or affinity chromatography. Monoclonal antibodies may be further screened or optimized for properties relating to specificity, avidity, half-life, immunogenicity, binding association, binding disassociation, or overall functional properties relative to being a treatment for infection. Thus, monoclonal antibodies may have alterations in the amino acid sequence of CDRs, including insertions, deletions, or substitutions with a conserved or non-conserved amino acid.

The immunogenicity of a particular immunogen composition can be enhanced by the use of non-specific stimulators of the immune response, known as adjuvants. Adjuvants that may be used in accordance with embodiments include, but are not limited to, IL-1, IL-2, IL-4, IL-7, IL-12, γ-interferon, GMCSF, BCG, aluminum hydroxide, MDP compounds, such as thur-MDP and nor-MDP, CGP (MTP-PE), lipid A, and monophosphoryl lipid A (MPL). Exemplary adjuvants may include complete Freund's adjuvant (a non-specific stimulator of the immune response containing killed Mycobacterium tuberculosis), incomplete Freund's adjuvants, and/or aluminum hydroxide adjuvant. In addition to adjuvants, it may be desirable to co-administer biologic response modifiers (BRM), such as but not limited to, Cimetidine (CIM; 1200 mg/d) (Smith/Kline, PA); low-dose Cyclophosphamide (CYP; 300 mg/m²) (Johnson/Mead, NJ), cytokines such as β-interferon, IL-2, or IL-12, or genes encoding proteins involved in immune helper functions, such as B-7.A phage-display system can be used to expand antibody molecule populations in vitro. Saiki, et al., Nature 324:163 (1986); Scharf et al., Science 233:1076 (1986); U.S. Pat. Nos. 4,683,195 and 4,683,202; Yang et al., J Mol Biol. 254:392 (1995); Barbas, III et al., Methods: Comp. Meth Enzymol. (1995) 8:94; Barbas, III et al., Proc Natl Acad Sci USA 88:7978 (1991).

### B. Fully Human Antibody Production

Methods are available for making fully human antibodies. Using fully human antibodies can minimize the immunogenic and allergic responses that may be caused by administering non-human monoclonal antibodies to humans as therapeutic agents. In one embodiment, human antibodies may be produced in a non-human transgenic animal, e.g., a transgenic mouse capable of producing multiple isotypes of human antibodies to protein (e.g., IgG, IgA, and/or IgE) by undergoing V-D-J recombination and isotype switching. Accordingly, this aspect applies to antibodies, antibody fragments, and pharmaceutical compositions thereof, but also non-human transgenic animals, B-cells, host cells, and hybridomas that produce monoclonal antibodies. Applications of humanized antibodies include, but are not limited to, detect a cell expressing an anticipated protein, either in vivo or in vitro, pharmaceutical preparations containing the antibodies of the present disclosure, and methods of treating disorders by administering the antibodies.

Fully human antibodies can be produced by immunizing transgenic animals (usually mice) that are capable of producing a repertoire of human antibodies in the absence of endogenous immunoglobulin production. Antigens for this purpose typically have six or more contiguous amino acids, and optionally are conjugated to a carrier, such as a hapten. See, for example, Jakobovits et al., Proc. Natl. Acad. Sci. USA 90:2551-2555 (1993); Jakobovits et al., Nature 362:255-258 (1993); Bruggermann et al., Year in Immunol. 7:33 (1993). In one example, transgenic animals are produced by incapacitating the endogenous mouse immunoglobulin loci encoding the mouse heavy and light immunoglobulin chains therein, and inserting into the mouse genome large fragments of human genome DNA containing loci that encode human heavy and light chain proteins. Partially modified animals, which have less than the full complement of human immunoglobulin loci, are then crossbred to obtain an animal having all of the desired immune system modifications. When administered an immunogen, these transgenic animals produce antibodies that are immunospecific for the immunogen but have human rather than murine amino acid sequences, including the variable regions. For further details of such methods, see, for example, International Patent Application Publication Nos. WO 96/33735 and WO 94/02602, which are hereby incorporated by reference in their entirety. Additional methods relating to transgenic mice for making human antibodies are described in U.S. Pat. Nos. 5,545,807; 6,713,610; 6,673,986; 6,162,963; 6,300,129; 6,255,458; 5,877,397; 5,874,299 and 5,545,806; in International Patent Application Publication Nos. WO 91/10741 and WO 90/04036; and in European Patent Nos. EP 546073B1 and EP 546073A1, all of which are hereby incorporated by reference in their entirety for all purposes.

The transgenic mice described above, referred to herein as “HuMAb” mice, contain a human immunoglobulin gene minilocus that encodes unrearranged human heavy (μ and γ) and κ light chain immunoglobulin sequences, together with targeted mutations that inactivate the endogenous μ and κ chain loci (Lonberg et al., Nature 368:856-859 (1994)). Accordingly, the mice exhibit reduced expression of mouse IgM or κ chains and in response to immunization, the introduced human heavy and light chain transgenes undergo class switching and somatic mutation to generate high affinity human IgG κ monoclonal antibodies (Lonberg et al., supra; Lonberg and Huszar, Intern. Ref. Immunol. 13:65-93 (1995); Harding and Lonberg, Ann. N.Y. Acad. Sci. 764:536-546 (1995)). The preparation of HuMAb mice is described in detail in Taylor et al., Nucl. Acids Res. 20:6287-6295 (1992); Chen et al., Int. Immunol. 5:647-656 (1993); Tuaillon et al., J. Immunol. 152:2912-2920 (1994); Lonberg et al., supra; Lonberg, Handbook of Exp. Pharmacol. 113:49-101 (1994); Taylor et al., Int. Immunol. 6:579-591 (1994); Lonberg and Huszar, Intern. Ref. Immunol. 13:65-93 (1995); Harding and Lonberg, Ann. N.Y. Acad. Sci. 764:536-546 (1995); Fishwild et al., Nat. Biotechnol. 14:845-851 (1996); the foregoing references are herein incorporated by reference in their entirety for all purposes. See further, U.S. Pat. Nos. 5,545,806; 5,569,825; 5,625,126; 5,633,425; 5,789,650; 5,877,397; 5,661,016; 5,814,318; 5,874,299; 5,770,429; and 5,545,807; as well as International Patent Application Publication Nos. WO 93/1227; WO 92/22646; and WO 92/03918, the disclosures of all of which are hereby incorporated by reference in their entirety for all purposes. Technologies utilized for producing human antibodies in these transgenic mice are disclosed also in WO 98/24893, and Mendez et al., Nat. Genetics 15:146-156 (1997), which are herein incorporated by reference. For example, the HCo7 and HCo12 transgenic mice strains can be used to generate human antibodies.

Using hybridoma technology, antigen-specific humanized monoclonal antibodies with the desired specificity can be produced and selected from the transgenic mice such as those described above. Such antibodies may be cloned and expressed using a suitable vector and host cell, or the antibodies can be harvested from cultured hybridoma cells. Fully human antibodies can also be derived from phage-display libraries (as disclosed in Hoogenboom et al., J. Mol. Biol. 227:381 (1991); and Marks et al., J. Mol. Biol. 222:581 (1991)). One such technique is described in International Patent Application Publication No. WO 99/10494 (herein incorporated by reference), which describes the isolation of high affinity and functional agonistic antibodies for MPL- and msk-receptors using such an approach.

### C. Antibody Fragments Production

Antibody fragments that retain the ability to recognize the antigen of interest will also find use herein. A number of antibody fragments are known in the art that comprise antigen-binding sites capable of exhibiting immunological binding properties of an intact antibody molecule and can be subsequently modified by methods known in the arts. Functional fragments, including only the variable regions of the heavy and light chains, can also be produced using standard techniques such as recombinant production or preferential proteolytic cleavage of immunoglobulin molecules. These fragments are known as Fv. See, e.g., Inbar et al., Proc. Nat. Acad. Sci. USA 69:2659-2662 (1972); Hochman et al., Biochem. 15:2706-2710 (1976); and Ehrlich et al., Biochem. 19:4091-4096 (1980).

Single-chain variable fragments (scFvs) may be prepared by fusing DNA encoding a peptide linker between DNAs encoding the two variable domain polypeptides (V_{L} and V_{H}). scFvs can form antigen-binding monomers, or they can form multimers (e.g., dimers, trimers, or tetramers), depending on the length of a flexible linker between the two variable domains (Kortt et al., Prot. Eng. 10:423 (1997); Kort et al., Biomol. Eng. 18:95-108 (2001)). By combining different V_{L}- and V_{H}-comprising polypeptides, one can form multimeric scFvs that bind to different epitopes (Kriangkum et al., Biomol. Eng. 18:31-40 (2001)). Antigen-binding fragments are typically produced by recombinant DNA methods known to those skilled in the art. Although the two domains of the Fv fragment, V_{L} and V_{H}, are coded for by separate genes, they can be joined using recombinant methods by a synthetic linker that enables them to be made as a single chain polypeptide (known as single chain Fv (sFv or scFv); sec e.g., Bird et al., Science 242:423-426 (1988); and Huston et al., Proc. Natl. Acad. Sci. USA 85:5879-5883 (1988). Design criteria include determining the appropriate length to span the distance between the C-terminus of one chain and the N-terminus of the other, wherein the linker is generally formed from small hydrophilic amino acid residues that do not tend to coil or form secondary structures. Suitable linkers generally comprise polypeptide chains of alternating sets of glycine and serine residues, and may include glutamic acid and lysine residues inserted to enhance solubility. Antigen-binding fragments are screened for utility in the same manner as intact antibodies. Such fragments include those obtained by amino-terminal and/or carboxy-terminal deletions, where the remaining amino acid sequence is substantially identical to the corresponding positions in the naturally occurring sequence deduced, for example, from a full-length cDNA sequence.

Antibodies may also be gencrated using peptide analogs of the epitopic determinants disclosed herein, which may consist of non-peptide compounds having properties analogous to those of the template peptide. These types of non-peptide compound are termed “peptide mimetics” or “peptidomimetics”. Fauchere, J. Adv. Drug Res. 15:29 (1986); Veber and Freidinger TINS p. 392 (1985); and Evans et al., J. Med. Chem. 30:1229 (1987). Liu et al. (2003) also describe “antibody like binding peptidomimetics” (ABiPs), which are peptides that act as pared-down antibodies and have certain advantages of longer serum half-life as well as less cumbersome synthesis methods. These analogs can be peptides, non-peptides or combinations of peptide and non-peptide regions. Fauchere, Adv. Drug Res. 15:29 (1986); Veber and Freidiner, TINS p. 392 (1985); and Evans et al., J. Med. Chem. 30:1229 (1987), which are incorporated herein by reference in their entirety for any purpose. Peptide mimetics that are structurally similar to therapeutically useful peptides may be used to produce a similar therapeutic or prophylactic effect. Such compounds are often developed with the aid of computerized molecular modeling. Generally, peptidomimetics of the disclosure are proteins that are structurally similar to an antibody displaying a desired biological activity, such as the ability to bind a protein, but have one or more peptide linkages optionally replaced by a linkage selected from: —CH2NH—, —CH2S—, —CH2-CH2-, —CH═CH— (cis and trans), —COCH2-, —CH(OH)CH2-, and —CH2SO— by methods well known in the art. Systematic substitution of one or more amino acids of a consensus sequence with a D-amino acid of the same type (e.g., D-lysine in place of L-lysine) may be used in certain embodiments of the disclosure to generate more stable proteins. In addition, constrained peptides comprising a consensus sequence or a substantially identical consensus sequence variation may be generated by methods known in the art (Rizo and Gierasch, Ann. Rev. Biochem. 61:387 (1992), incorporated herein by reference), for example, by adding internal cysteine residues capable of forming intramolecular disulfide bridges which cyclize the peptide.

Once generated, a phage display library can be used to improve the immunological binding affinity of the Fab molecules using known techniques. See, e.g., Figini et al., J. Mol. Biol. 239:68 (1994). The coding sequences for the heavy and light chain portions of the Fab molecules selected from the phage display library can be isolated or synthesized and cloned into any suitable vector or replicon for expression. Any suitable expression system can be used.

### VII. Administration of Therapeutic Compositions

Embodiments of the disclosure encompass methods of treating a medical condition in an individual caused indirectly or directly by the presence of TdT or a TdT peptide associated with HLA-A02 on the surface of cells in the individual, comprising the step of administering a therapeutically effective amount of any composition encompassed herein to the individual, including cells, polynucleotides, polypeptides, and so forth. In specific embodiments, the medical condition is cancer of any kind, including a hematological malignancy or a solid tumor. The hematological malignancy may be leukemia or lymphoma. The leukemia may be acute myeloid leukemia (AML), chronic myeloid (or myelogenous) leukemia (CML); acute lymphocytic (or lymphoblastic) leukemia (ALL); or chronic lymphocytic leukemia. The lymphoma may be Hodgkin lymphoma, non-Hodgkin lymphoma, chronic lymphocytic leukemia (CLL), or small lymphocytic lymphoma (SLL). In various embodiments, the medical condition is cancer and wherein the individual is administered a therapeutically effective amount of cells comprising recombinant receptor (such as a TCR or STAR) comprising an scFv specific a TdT peptide and wherein the individual is administered a therapeutically effective amount of cells comprising a BiTE comprising an scFv specific for a TdT peptide. In some cases, the cells comprising the recombinant receptor (TCR and/or STAR) and the cells comprising the BiTE are the same cells. In some cases, the cells comprising the recombinant receptor (TCR and/or STAR) and the cells comprising the BiTE are different cells. In specific embodiments, the administration of the compositions occurs prior to and/or following onset of one or more symptoms of a medical condition, including cancer.

The therapy provided herein may comprise administration of a combination of therapeutic agents, such as a first cancer therapy and a second cancer therapy. The therapies may be administered in any suitable manner known in the art. For example, the first and second cancer treatment may be administered sequentially (at different times) or concurrently (at the same time). In some embodiments, the first and second cancer treatments are administered in a separate composition. In some embodiments, the first and second cancer treatments are in the same composition.

In some embodiments, the cells expressing the TdT-specific cTCR and/or STAR therapy and the TdT-specific BiTE therapy are administered substantially simultaneously. In some embodiments, the TdT-specific cTCR and/or STAR therapy and the TdT-specific BiTE therapy are administered sequentially. In some embodiments, the TdT-specific cTCR and/or STAR therapy, the TdT-specific BiTE therapy, and a third or more therapy are administered sequentially. In some embodiments, the TdT-specific cTCR and/or STAR therapy is administered before administering the TdT-specific BiTE therapy. In some embodiments, the TdT-specific cTCR and/or STAR therapy is administered after administering the TdT-specific BiTE therapy. In various embodiments, the TdT-specific cTCR and/or STAR therapy and the TdT-specific BiTE therapy are administered substantially simultaneously because the cells that express the TdT-specific cTCR and/or STAR are the cells that express and secrete the soluble TdT-specific BiTE molecules. In some embodiments, the TdT-specific cTCR and/or STAR therapy and the TdT-specific BiTE therapy are administered substantially simultaneously but the cells that express the TdT-specific cTCR and/or STAR are not the cells that express and secrete the soluble TdT-specific BiTE molecules. In some embodiments, the TdT-specific cTCR and/or STAR therapy and the TdT-specific BiTE therapy are administered at different times and the cells that express the TdT-specific cTCR and/or STAR are not the cells that express and secrete the soluble TdT-specific BiTE molecules.

Embodiments of the disclosure relate to compositions and methods comprising therapeutic compositions. The different therapies may be administered in one composition or in more than one composition, such as 2 compositions, 3 compositions, or 4 compositions. Various combinations of the agents may be employed.

The therapeutic agents of the disclosure may be administered by the same route of administration or by different routes of administration. In some embodiments, the cancer therapy is administered intravenously, intramuscularly, subcutaneously, topically, orally, transdermally, intraperitoneally, intraorbitally, by implantation, by inhalation, intrathecally, intraventricularly, or intranasally. In some embodiments, the antibiotic is administered intravenously, intramuscularly, subcutaneously, topically, orally, transdermally, intraperitoneally, intraorbitally, by implantation, by inhalation, intrathecally, intraventricularly, or intranasally. The appropriate dosage may be determined based on the type of disease to be treated, severity and course of the disease, the clinical condition of the individual, the individual's clinical history and response to the treatment, and the discretion of the attending physician.

The treatments may include various “unit doses.” Unit dose is defined as containing a predetermined-quantity of the therapeutic composition. The quantity to be administered, and the particular route and formulation, is within the skill of determination of those in the clinical arts. A unit dose need not be administered as a single injection but may comprise continuous infusion over a set period of time. In some embodiments, a unit dose comprises a single administrable dose.

In some embodiments, the TdT-specific cTCR and/or STAR therapy comprises a TdT-specific cTCR and/or STAR protein, a nucleic acid encoding for the TdT-specific cTCR and/or STAR protein, a vector comprising the nucleic acid encoding for the TdT-specific cTCR and/or STAR protein, and/or a cell comprising the TdT-specific cTCR and/or STAR protein. In some embodiments, a single dose of the TdT-specific cTCR and/or STAR protein therapy is administered. In some embodiments, multiple doses of the TdT-specific cTCR and/or STAR protein are administered. Such administrations may or may not also comprise BiTE therapy that comprises a BiTE protein, a nucleic acid encoding for the BiTE protein, a vector comprising the nucleic acid encoding for the BiTE protein, and/or a cell comprising the BiTE protein. The cells may or may not be the same cells that comprise the TdT-specific cTCR and/or STAR protein.

In some embodiments, the TdT-specific cTCR and/or STAR protein and/or BiTE protein are administered at a dose of between 1 mg/kg and 5000 mg/kg. In some embodiments, the TdT-specific cTCR and/or STAR protein and/or BiTE are administered at a dose of at least, at most, or about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 600, 700, 800, 900, 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200, 4300, 4400, 4500, 4600, 4700, 4800, 4900, or 5000 mg/kg.

The quantity to be administered, both according to number of treatments and unit dose, depends on the treatment effect desired. An effective dose is understood to refer to an amount necessary to achieve a particular effect. In the practice in certain embodiments, it is contemplated that doses in the range from 10 mg/kg to 200 mg/kg are efficacious. Thus, it is contemplated that doses include doses of about 0.1, 0.5, 1, 5, 10, 15, 20, 25, 30, 35, 40, 45, 50, 55, 60, 65, 70, 75, 80, 85, 90, 100, 105, 110, 115, 120, 125, 130, 135, 140, 145, 150, 155, 160, 165, 170, 175, 180, 185, 190, 195, and 200, 300, 400, 500, 1000 μg/kg, mg/kg, μg/day, or mg/day or any range derivable therein. Furthermore, such doses can be administered at multiple times during a day, and/or on multiple days, weeks, or months.

In certain embodiments, the effective dose of the pharmaceutical composition is one that can provide a blood level of about 1 μM to 150 μM. In another embodiment, the effective dose provides a blood level of about 4 μM to 100 μM.; or about 1 μM to 100 μM; or about 1 μM to 50 μM; or about 1 μM to 40 μM; or about 1 μM to 30 μM; or about 1 μM to 20μM; or about 1 μM to 10 μM; or about 10 μM to 150 μM; or about 10 μM to 100 μM; or about 10 μM to 50 μM; or about 25 μM to 150 μM; or about 25 μM to 100 μM; or about 25 μM to 50 M; or about 50 M to 150 μM; or about 50 μM to 100 μM (or any range derivable therein). In other embodiments, the dose can provide the following blood level of the agent that results from a therapeutic agent being administered to a subject: about, at least about, or at most about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 μM or any range derivable therein. In certain embodiments, the therapeutic agent that is administered to a subject is metabolized in the body to a metabolized therapeutic agent, in which case the blood levels may refer to the amount of that agent. Alternatively, to the extent the therapeutic agent is not metabolized by a subject, the blood levels discussed herein may refer to the unmetabolized therapeutic agent.

Precise amounts of the therapeutic composition also depend on the judgment of the practitioner and are peculiar to each individual. Factors affecting dose include physical and clinical state of the patient, the route of administration, the intended goal of treatment (alleviation of symptoms versus cure) and the potency, stability and toxicity of the particular therapeutic substance or other therapies a subject may be undergoing.

It will be understood by those skilled in the art and made aware that dosage units of μg/kg or mg/kg of body weight can be converted and expressed in comparable concentration units of μg/ml or mM (blood levels), such as 4 μM to 100 μM. It is also understood that uptake is species and organ/tissue dependent. The applicable conversion factors and physiological assumptions to be made concerning uptake and concentration measurement are well-known and would permit those of skill in the art to convert one concentration measurement to another and make reasonable comparisons and conclusions regarding the doses, efficacies and results described herein.

In certain instances, it will be desirable to have multiple administrations of the composition, e.g., 2, 3, 4, 5, 6 or more administrations. The administrations can be at 1, 2, 3, 4, 5, 6, 7, 8, to 5, 6, 7, 8, 9, 10, 11, or 12 week intervals, including all ranges there between.

The phrases “pharmaceutically acceptable” or “pharmacologically acceptable” refer to molecular entities and compositions that do not produce an adverse, allergic, or other untoward reaction when administered to an animal or human. As used herein, “pharmaceutically acceptable carrier” includes any and all solvents, dispersion media, coatings, anti-bacterial and anti-fungal agents, isotonic and absorption delaying agents, and the like. The use of such media and agents for pharmaceutical active substances is well known in the art. Except insofar as any conventional media or agent is incompatible with the active ingredients, its use in immunogenic and therapeutic compositions is contemplated. Supplementary active ingredients, such as other anti-infective agents and vaccines, can also be incorporated into the compositions.

The active compounds can be formulated for parenteral administration, e.g., formulated for injection via the intravenous, intramuscular, subcutaneous, or intraperitoneal routes. Typically, such compositions can be prepared as either liquid solutions or suspensions; solid forms suitable for use to prepare solutions or suspensions upon the addition of a liquid prior to injection can also be prepared; and, the preparations can also be emulsified.

The pharmaceutical forms suitable for injectable use include sterile aqueous solutions or dispersions; formulations including, for example, aqueous propylene glycol; and sterile powders for the extemporaneous preparation of sterile injectable solutions or dispersions. In all cases the form must be sterile and must be fluid to the extent that it may be easily injected. It also should be stable under the conditions of manufacture and storage and must be preserved against the contaminating action of microorganisms, such as bacteria and fungi.

The proteinaceous compositions may be formulated into a neutral or salt form. Pharmaceutically acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

A pharmaceutical composition can include a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion, and by the use of surfactants. The prevention of the action of microorganisms can be brought about by various anti-bacterial and anti-fungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various other ingredients enumerated above, as required, followed by filtered sterilization or an equivalent procedure. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques, which yield a powder of the active ingredient, plus any additional desired ingredient from a previously sterile-filtered solution thereof.

Administration of the compositions will typically be via any common route. This includes, but is not limited to oral, or intravenous administration. Alternatively, administration may be by orthotopic, intradermal, subcutaneous, intramuscular, intraperitoneal, or intranasal administration. Such compositions would normally be administered as pharmaceutically acceptable compositions that include physiologically acceptable carriers, buffers or other excipients.

Upon formulation, solutions will be administered in a manner compatible with the dosage formulation and in such amount as is therapeutically or prophylactically effective. The formulations are easily administered in a variety of dosage forms, such as the type of injectable solutions described above.

### II. TdT-specific Cellular Therapies

### A. Cell Culture

In some embodiments, TdT-related therapeutic cells (which may express a TdT-specific chimeric TCR and/or TdT-specific STAR and/or TdT-specific BiTE) may be cultured for at least between about 10 days and about 40 days, for at least between about 15 days and about 35 days, for at least between about 15 days and 21 days, such as for at least about 15, 16, 17, 18, 19 or 21 days. In some embodiments, the cells of the disclosure may be cultured for no longer than 60 days, or no longer than 50 days, or no longer than 45 days. The cells may be cultured for 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 days. The cells may be cultured in the presence of a liquid culture medium. Typically, the medium may comprise a basal medium formulation as known in the art. Many basal media formulations can be used to culture cells herein, including but not limited to Eagle's Minimum Essential Medium (MEM), Dulbecco's Modified Eagle's Medium (DMEM), alpha modified Minimum Essential Medium (alpha-MEM), Basal Medium Essential (BME), Iscove's Modified Dulbecco's Medium (IMDM), BGJb medium, F-12 Nutrient Mixture (Ham), Liebovitz L-15, DMEM/F-12, Essential Modified Eagle's Medium (EMEM), RPMI-1640, and modifications and/or combinations thereof. Compositions of the above basal media are generally known in the art, and it is within the skill of one in the art to modify or modulate concentrations of media and/or media supplements as necessary for the cells cultured. In some embodiments, a culture medium formulation may be explants medium (CEM) which is composed of IMDM supplemented with 10% fetal bovine serum (FBS), 100 U/ml penicillin G, 100 μg/ml streptomycin and 2 mmol/L L-glutamine. Other embodiments may employ further basal media formulations, such as chosen from the ones above.

Any medium capable of supporting cells in vitro may be used to culture the cells. Media formulations that can support the growth of cells include, but are not limited to, Dulbecco's Modified Eagle's Medium (DMEM), alpha modified Minimal Essential Medium (αMEM), and Roswell Park Memorial Institute Media 1640 (RPMI Media 1640) and the like. Typically, up to 20% fetal bovine serum (FBS) or 1-20% horse serum is added to the above medium in order to support the growth of cells. A defined medium, however, also can be used if the growth factors, cytokines, and hormones necessary for culturing cells are provided at appropriate concentrations in the medium. Media useful in the methods of the disclosure may comprise one or more compounds of interest, including, but not limited to, antibiotics, mitogenic compounds, or differentiation compounds useful for the culturing of cells. The cells may be grown at temperatures between 27° C. to 40° C., such as 31° C. to 37° C., and may be in a humidified incubator. The carbon dioxide content may be maintained between 2% to 10% and the oxygen content may be maintained between 1% and 22%. The disclosure, however, should in no way be construed to be limited to any one method of isolating and culturing cells. Rather, any method of isolating and culturing cells should be construed to be included in the present disclosure.

For use in the cell culture, media can be supplied with one or more further components. For example, additional supplements can be used to supply the cells with the necessary trace elements and substances for optimal growth and expansion. Such supplements include insulin, transferrin, selenium salts, and combinations thereof. These components can be included in a salt solution such as, but not limited to, Hanks' Balanced Salt Solution (HBSS), Earle's Salt Solution. Further antioxidant supplements may be added, e.g., β-mercaptoethanol. While many media already contain amino acids, some amino acids may be supplemented later, e.g., L-glutamine, which is known to be less stable when in solution. A medium may be further supplied with antibiotic and/or antimycotic compounds, such as, typically, mixtures of penicillin and streptomycin, and/or other compounds, exemplified but not limited to, amphotericin, ampicillin, gentamicin, bleomycin, hygromycin, kanamycin, mitomycin, mycophenolic acid, nalidixic acid, neomycin, nystatin, paromomycin, polymyxin, puromycin, rifampicin, spectinomycin, tetracycline, tylosin, and zeocin. Also contemplated is supplementation of cell culture medium with mammalian plasma or sera. Plasma or sera often contain cellular factors and components that are necessary for viability and expansion. The use of suitable serum replacements is also contemplated.

Reference to particular buffers, media, reagents, cells, culture conditions and the like, or to some subclass of same, is not intended to be limiting, but should be read to include all such related materials that one of ordinary skill in the art would recognize as being of interest or value in the particular context in which that discussion is presented. For example, it is often possible to substitute one buffer system or culture medium for another, such that a different but known way is used to achieve the same goals as those to which the use of a suggested method, material or composition is directed. In particular embodiments, cells are cultured in a cell culture system comprising a cell culture medium, preferably in a culture vessel, in particular a cell culture medium supplemented with a substance suitable and determined for protecting the cells from in vitro aging and/or inducing in an unspecific or specific reprogramming.

### B. Cell Generation

Certain methods of the disclosure concern culturing the cells obtained from human tissue samples. In particular embodiments of the present disclosure, cells are plated onto a substrate that allows for adherence of cells thereto. This may be carried out, for example, by plating the cells in a culture plate that displays one or more substrate surfaces compatible with cell adhesion. When the one or more substrate surfaces contact the suspension of cells (e.g., suspension in a medium) introduced into the culture system, cell adhesion between the cells and the substrate surfaces may ensue. Accordingly, in certain embodiments cells are introduced into a culture system that features at least one substrate surface that is generally compatible with adherence of cells thereto, such that the plated cells can contact the said substrate surface, such embodiments encompass plating onto a substrate, which allows adherence of cells thereto.

Cells of the present disclosure may be identified and characterized by their expression of specific marker proteins, such as cell-surface markers. Detection and isolation of these cells can be achieved, for example, through flow cytometry, ELISA, and/or magnetic beads. Reverse-transcription polymerase chain reaction (RT-PCR) may be used to quantify cell-specific genes and/or to monitor changes in gene expression in response to differentiation.

### C. Chimeric T-Cell Receptors and Synthetic T Cell Receptor (TCR) and Antigen Receptors (STARs)

Embodiments of the disclosure utilize chimeric receptors (including T-cell receptors and others) that bind to the TdT peptide. T-cell receptors comprise two different polypeptide chains, termed the T-cell receptor α (TCRα) and β (TCRβ) chains, linked by a disulfide bond. These α:β heterodimers are very similar in structure to the Fab fragment of an immunoglobulin molecule, and they account for antigen recognition by most T cells. A minority of T cells bear an alternative, but structurally similar, receptor made up of a different pair of polypeptide chains designated γ and δ. Both types of T cell receptor differ from the membrane-bound immunoglobulin that serves as the B-cell receptor: a T cell receptor has only one antigen-binding site, whereas a B-cell receptor has two, and T-cell receptors are never secreted, whereas immunoglobulin can be secreted as antibody.

Both chains of the T-cell receptor have an amino-terminal variable (V) region with homology to an immunoglobulin V domain, a constant (C) region with homology to an immunoglobulin C domain, and a short hinge region containing a cysteine residue that forms the interchain disulfide bond. Each chain spans the lipid bilayer by a hydrophobic transmembrane domain, and ends in a short cytoplasmic tail.

Embodiments of the disclosure relate to engineered T cell receptors that bind a TdT peptide of the disclosure, such as a peptide of SEQ ID NO:3. The term “engineered” refers to T cell receptors that in at least some cases have TCR variable regions grafted onto TCR constant regions to make a chimeric polypeptide that binds to TdT peptide antigens of the disclosure. In certain embodiments, the TCR comprises intervening sequences that are used for cloning, enhanced expression, detection, or for therapeutic control of the construct, but are not present in endogenous TCRs, such as multiple cloning sites, linker, hinge sequences, modified hinge sequences, modified transmembrane sequences, a detection polypeptide or molecule, or therapeutic controls that may allow for selection or screening of cells comprising the TCR.

In some embodiments, the TCR comprises non-TCR sequences. Accordingly, certain embodiments relate to TCRs with sequences that are not from a TCR gene. In some embodiments, the TCR is chimeric, in that it contains sequences normally found in a TCR gene, but contains sequences from at least two TCR genes that are not necessarily found together in nature.

In some embodiments of the disclosure, there are engineered receptors that bind a TdT peptide of the disclosure, such as a peptide of SEQ ID NO:3 that in at least some cases comprise the V_{H} and V_{L} domains on separate chains but that still comprise a TCR alpha chain and a TCR beta chain (STAR). In certain embodiments, the STAR comprises intervening sequences that are used for cloning, enhanced expression, detection, or for therapeutic control of the construct, but are not present in endogenous TCRs, such as multiple cloning sites, linker, hinge sequences, modified hinge sequences, modified transmembrane sequences, a detection polypeptide or molecule, or therapeutic controls that may allow for selection or screening of cells comprising the STAR.

In some embodiments, the STAR comprises non-TCR sequences. Accordingly. certain embodiments relate to STARs with sequences that are not from a TCR gene. In some embodiments, the STAR is chimeric, in that it contains sequences normally found in a TCR gene, but contains sequences from at least two TCR genes that are not necessarily found together in nature.

### D. Peptides and Polypeptide Inhibitors

As used herein, a “protein” or “polypeptide” refers to a molecule comprising at least five amino acid residues. As used herein, the term “wild-type” refers to the endogenous version of a molecule that occurs naturally in an organism. In some embodiments, wild-type versions of a protein or polypeptide are employed, however, in many embodiments of the disclosure, a modified protein or polypeptide is employed to generate an immune response. The terms described above may be used interchangeably. A “modified protein” or “modified polypeptide” or a “variant” refers to a protein or polypeptide whose chemical structure, particularly its amino acid sequence, is altered with respect to the wild-type protein or polypeptide. In some embodiments, a modified/variant protein or polypeptide has at least one modified activity or function (recognizing that proteins or polypeptides may have multiple activities or functions), such as an altered TCR alpha or beta chain. It is specifically contemplated that a modified/variant protein or polypeptide may be altered with respect to one activity or function yet retain a wild-type activity or function in other respects, such as immunogenicity.

Where a protein is specifically mentioned herein, it is in general a reference to a native (wild-type) or recombinant (modified) protein or, optionally, a protein in which any signal sequence has been removed. The protein may be isolated directly from the organism of which it is native, produced by recombinant DNA/exogenous expression methods, or produced by solid-phase peptide synthesis (SPPS) or other in vitro methods. In particular embodiments, there are isolated nucleic acid segments and recombinant vectors incorporating nucleic acid sequences that encode a polypeptide (e.g., an antibody or fragment thereof). The term “recombinant” may be used in conjunction with a polypeptide or the name of a specific polypeptide, and this generally refers to a polypeptide produced from a nucleic acid molecule that has been manipulated in vitro or that is a replication product of such a molecule.

In certain embodiments the size of a peptide or protein of the disclosure may comprise, but is not limited to, 5, 6, 7, 8, 9, 10, 11, 12, 13, 14, 15, 16, or 17 amino acid residues or greater, and any range derivable therein. It is contemplated that polypeptides may be mutated by truncation, rendering them shorter than their corresponding wild-type form, also, they might be altered by fusing or conjugating a heterologous protein or polypeptide sequence with a particular function (e.g., for targeting or localization, for enhanced immunogenicity, for purification purposes, etc.). As used herein, the term “domain” refers to any distinct functional or structural unit of a protein or polypeptide, and generally refers to a sequence of amino acids with a structure or function recognizable by one skilled in the art.

The polypeptides and peptides of the disclosure may include 1, 2, 3, 4, 5, 6, 7, 8, 9, or 10 (or any derivable range therein) or more variant amino acids substitutions or be at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% (or any derivable range therein) similar, identical, or homologous with at least, or at most 3, 4, 5, 6, 7, 8, 9, 10, 11, 12, 13, 14, 15, 16, or 17 contiguous amino acids or nucleic acids, or any range derivable therein, of SEQ ID NOs: 4-19.

In some embodiments, the peptide or polypeptide may comprise amino acids 1 to 2, 3, 4, 5, 6, 7, 8, 9, 10, 11, 12, 13, 14, 15, 16, or 17, (or any derivable range therein) of SEQ ID NOS: 3, 5, 7, 9, 11, 13, 15, or 17.

In some embodiments, the peptide or polypeptide may comprise 1, 2, 3, 4, 5, 6, 7, 8, 9, 10, 11, 12, 13, 14, 15, 16, or 17, (or any derivable range therein) contiguous amino acids of SEQ ID NOs: 3, 5, 7, 9, 11, 13, 15, or 17.

In some embodiments, the peptide or polypeptide may comprise, may comprise at least, may comprise at most 1, 2, 3, 4, 5, 6, 7, 8, 9, 10, 11, 12, 13, 14, 15, 16, or 17 (or any derivable range therein) contiguous amino acids of SEQ ID NOS: 3, 5, 7, 9, 11, 13, 15, or 17 that are at least, at most, or exactly 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% (or any derivable range therein) similar, identical, or homologous with one of SEQ ID NOS: 3, 5, 7, 9, 11, 13, 15, or 17.

In some aspects there is a peptide or polypeptide starting at position 1, 2, 3, 4, 5, 6, 7, 8, 9, or 10 of any of SEQ ID NOS: 3, 5, 7, 9, 11, 13, 15, or 17 and comprising, comprising at least, or comprising at most 2, 3, 4, 5, 6, 7, 8, 9, 10, 11, 12, 13, 14, 15, 16, or 17 (or any derivable range therein) contiguous amino acids or nucleotides of any of SEQ ID NOS: 3, 5, 7, 9, 11, 13, 15, or 17.

The peptide or polypeptide may comprise, may comprise at least, or may comprise at most 1, 2, 3, 4, 5, 6, 7, 8, 9, or 10 (or any derivable range therein) amino acid substitutions relative to SEQ ID NOS: 3, 5, 7, 9, 11, 13, 15, or 17. The peptide or polypeptide may comprise, may comprise at least, or may comprise at most 1, 2, 3, 4, 5, 6, 7, 8, 9, or 10 (or any derivable range therein) amino acid substitutions, and the substitution(s) may be at position 1, 2, 3, 4, 5, 6, 7, 8, 9, 10, 11, 12, 13, 14, 15, 16, and/or 17 relative to SEQ ID NOS: 3, 5, 7, 9, 11, 13, 15, or 17. The substitution at position(s), 2, 3, 4, 5, 6, 7, 8, 9, 10, 11, 12, 13, 14, 15, 16, and/or 17 may be with an alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, or valine.

The nucleotide as well as the protein, polypeptide, and peptide sequences for various genes have been previously disclosed, and may be found in the recognized computerized databases. Two commonly used databases are the National Center for Biotechnology Information's Genbank® and GenPept® databases (on the World Wide Web at ncbi.nlm.nih.gov/) and The Universal Protein Resource (UniProt; on the World Wide Web at uniprot.org). The coding regions for these genes may be amplified and/or expressed using the techniques disclosed herein or as would be known to those of ordinary skill in the art.

It is contemplated that in compositions of the disclosure, there is between about 0.001 mg and about 10 mg of total polypeptide, peptide, and/or protein per ml. The concentration of protein in a composition can be about, at least about or at most about 0.001, 0.010, 0.050, 0.1, 0.2, 0.3, 0.4, 0.5, 0.6, 0.7, 0.8, 0.9, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10.0 mg/ml or more (or any range derivable therein).

### VIII. Cells Expressing the TdT-Specific Reagents

In various embodiments, host cells are utilized into which at least one recombinant expression vector expressing the TdT-specific cTCR and/or TdT-specific STAR and/or the TDT-specific BiTE has been introduced. There may be separate expression vectors for the TdT-specific cTCR and the TdT-specific STAR and the TDT-specific BiTE, or they may be expressed from the same one or two vectors. The reagents can be expressed in a variety of cell types, including any type of immune effector cells. Certain embodiments relate to cells comprising polypeptides or nucleic acids of the disclosure. In some embodiments the cell is an immune cell including a T cell. “T cell” includes all types of immune cells including T-helper cells, invariant natural killer T (iNKT) cells, cytotoxic T cells, T-regulatory cells (Treg) gamma-delta T cells, natural-killer (NK) cells, and neutrophils. The T cell may refer to a CD4+ or CD8+ T cell. In specific embodiments, the cells are T cells, NK T cells, NK cells, macrophages, B cells, or a mixture thereof, for example. An expression construct encoding the TdT-specific cTCR and/or the TDT-specific BiTE can be transfected into cells according to a variety of methods known in the art. Vector DNA can be introduced into prokaryotic or eukaryotic cells via conventional transformation or transfection techniques. Some vectors may employ control sequences that allow it to be replicated and/or expressed in both prokaryotic and eukaryotic cells. In certain aspects, the antibody expression construct can be placed under control of a promoter that is linked to T-cell activation, such as one that is controlled by NFAT-1 or NF-κB, both of which are transcription factors that can be activated upon T-cell activation. Control of antibody expression allows T cells, such as tumor-targeting T cells, to sense their surroundings and perform real-time modulation of cytokine signaling, both in the T cells themselves and in surrounding endogenous immune cells. One of skill in the art would understand the conditions under which to incubate host cells to maintain them and to permit replication of a vector. Also understood and known are techniques and conditions that would allow large-scale production of vectors, as well as production of the nucleic acids encoded by vectors and their cognate polypeptides, proteins, or peptides.

For stable transfection of mammalian cells, it is known, depending upon the expression vector and transfection technique used, only a small fraction of cells may integrate the foreign DNA into their genome. In order to identify and select these integrants, a selectable marker (e.g., for resistance to antibiotics) is generally introduced into the host cells along with the gene of interest. Cells stably transfected with the introduced nucleic acid can be identified by drug selection (e.g., cells that have incorporated the selectable marker gene will survive, while the other cells die), among other methods known in the arts.

### III. Formulations and Culture of the Cells

In particular embodiments, the TT-specific reagent-expressing cells of the disclosure may be specifically formulated and/or they may be cultured in a particular medium. The cells may be formulated in such a manner as to be suitable for delivery to a recipient without deleterious effects.

The medium in certain aspects can be prepared using a medium used for culturing animal cells as their basal medium, such as any of AIM V, X-VIVO-15, NeuroBasal, EGM2, TeSR, BME, BGJb, CMRL 1066, Glasgow MEM, Improved MEM Zinc Option, IMDM, Medium 199, Eagle MEM, αMEM, DMEM, Ham, RPMI-1640, and Fischer's media, as well as any combinations thereof, but the medium may not be particularly limited thereto as far as it can be used for culturing animal cells. Particularly, the medium may be xeno-free or chemically defined.

The medium can be a serum-containing or serum-free medium, or xeno-free medium. From the aspect of preventing contamination with heterogeneous animal-derived components, serum can be derived from the same animal as that of the stem cell(s). The serum-free medium refers to medium with no unprocessed or unpurified serum and accordingly, can include medium with purified blood-derived components or animal tissue-derived components (such as growth factors).

The medium may contain or may not contain any alternatives to serum. The alternatives to serum can include materials which appropriately contain albumin (such as lipid-rich albumin, bovine albumin, albumin substitutes such as recombinant albumin or a humanized albumin, plant starch, dextrans and protein hydrolysates), transferrin (or other iron transporters), fatty acids, insulin, collagen precursors, trace elements, 2-mercaptoethanol, 3′-thiolgiycerol, or equivalents thereto. The alternatives to serum can be prepared by the method disclosed in International Publication No. 98/30679, for example (incorporated herein in its entirety). Alternatively, any commercially available materials can be used for more convenience. The commercially available materials include knockout Serum Replacement (KSR), Chemically-defined Lipid concentrated (Gibco), and Glutamax (Gibco).

In certain embodiments, the medium may comprise one, two, three, four, five, six, seven, eight, nine, ten, 11, 12, 13, 14, 15, 16, 17, 18, 19, 20 or more of the following: Vitamins such as biotin; DL Alpha Tocopherol Acetate; DL Alpha-Tocopherol; Vitamin A (acetate); proteins such as BSA (bovine serum albumin) or human albumin, fatty acid free Fraction V; Catalase; Human Recombinant Insulin; Human Transferrin; Superoxide Dismutase; Other Components such as Corticosterone; D-Galactose; Ethanolamine HCl; Glutathione (reduced); L-Carnitine HCl; Linoleic Acid; Linolenic Acid; Progesterone; Putrescinc 2HCl; Sodium Selenite; and/or T3 (triodo-I-thyronine). In specific embodiments, one or more of these may be explicitly excluded.

In some embodiments, the medium further comprises vitamins. In some embodiments, the medium comprises 1, 2, 3, 4, 5, 6, 7, 8, 9, 10, 11, 12, or 13 of the following (and any range derivable therein): biotin, DL alpha tocopherol acetate, DL alpha-tocopherol, vitamin A, choline chloride, calcium pantothenate, pantothenic acid, folic acid nicotinamide, pyridoxine, riboflavin, thiamine, inositol, vitamin B12, or the medium includes combinations thereof or salts thereof. In some embodiments, the medium comprises or consists essentially of biotin, DL alpha tocopherol acetate, DL alpha-tocopherol, vitamin A, choline chloride, calcium pantothenate, pantothenic acid, folic acid nicotinamide, pyridoxine, riboflavin, thiamine, inositol, and vitamin B12. In some embodiments, the vitamins include or consist essentially of biotin, DL alpha tocopherol acetate, DL alpha-tocopherol, vitamin A, or combinations or salts thereof. In some embodiments, the medium further comprises proteins. In some embodiments, the proteins comprise albumin or bovine serum albumin, a fraction of BSA, catalase, insulin, transferrin, superoxide dismutase, or combinations thereof. In some embodiments, the medium further comprises one or more of the following: corticosterone, D-Galactose, ethanolamine, glutathione, L-carnitine, linoleic acid, linolenic acid, progesterone, putrescine, sodium selenite, or triodo-I-thyronine, or combinations thereof. In some embodiments, the medium comprises one or more of the following: a B-27® supplement, xeno-free B-27® supplement, GS21™ supplement, or combinations thereof. In some embodiments, the medium comprises or further comprises amino acids, monosaccharides, inorganic ions. In some embodiments, the amino acids comprise arginine, cystine, isoleucine, leucine, lysine, methionine, glutamine, phenylalanine, threonine, tryptophan, histidine, tyrosine, or valine, or combinations thereof. In some embodiments, the inorganic ions comprise sodium, potassium, calcium, magnesium, nitrogen, or phosphorus, or combinations or salts thereof. In some embodiments, the medium further comprises one or more of the following: molybdenum, vanadium, iron, zinc, selenium, copper, or manganese, or combinations thereof. In certain embodiments, the medium comprises or consists essentially of one or more vitamins discussed herein and/or one or more proteins discussed herein, and/or one or more of the following: corticosterone, D-Galactose, ethanolamine, glutathione, L-carnitine, linoleic acid, linolenic acid, progesterone, putrescine, sodium selenite, or triodo-I-thyronine, a B-27® supplement, xeno-free B-27® supplement, GS21™ supplement, an amino acid (such as arginine, cystine, isoleucine, leucine, lysine, methionine, glutamine, phenylalanine, threonine, tryptophan, histidine, tyrosine, or valine), monosaccharide, inorganic ion (such as sodium, potassium, calcium, magnesium, nitrogen, and/or phosphorus) or salts thereof, and/or molybdenum, vanadium, iron, zinc, selenium, copper, or manganese. In specific embodiments, one or more of these may be explicitly excluded.

The medium can also contain one or more externally added fatty acids or lipids, amino acids (such as non-essential amino acids), vitamin(s), growth factors, cytokines, antioxidant substances, 2-mercaptoethanol, pyruvic acid, buffering agents, and/or inorganic salts. In specific embodiments, one or more of these may be explicitly excluded.

One or more of the medium components may be added at a concentration of at least, at most, or about 0.1, 0.5, 1, 2, 3, 4, 5, 10, 15, 20, 25, 30, 35, 40, 45, 50, 55, 60, 65, 70, 75, 80, 85, 90, 95, 100, 150, 180, 200, 250 ng/L, ng/ml, μg/ml, mg/ml, or any range derivable therein.

In specific embodiments, the cells of the disclosure are specifically formulated. They may or may not be formulated as a cell suspension. In specific cases they are formulated in a single dose form. They may be formulated for systemic or local administration. In some cases the cells are formulated for storage prior to use, and the cell formulation may comprise one or more cryopreservation agents, such as DMSO (for example, in 5% DMSO). The cell formulation may comprise albumin, including human albumin, with a specific formulation comprising 2.5% human albumin. The cells may be formulated specifically for intravenous administration; for example, they are formulated for intravenous administration over less than one hour. In particular embodiments the cells are in a formulated cell suspension that is stable at room temperature for 1, 2, 3, or 4 hours or more from time of thawing.

In particular embodiments, the cells of the disclosure comprise an exogenous TCR, which may be of a defined antigen specificity. In some embodiments, the TCR can be selected based on absent or reduced alloreactivity to the intended recipient. In the example where the exogenous TCR is non-alloreactive, during T cell differentiation the exogenous TCR suppresses rearrangement and/or expression of endogenous TCR loci through a developmental process called allelic exclusion, resulting in T cells that express only the non-alloreactive exogenous TCR and are thus non-alloreactive. In some embodiments, the choice of exogenous TCR may not necessarily be defined based on lack of alloreactivity. In some embodiments, the endogenous TCR genes have been modified by genome editing so that they do not express a protein. Methods of gene editing such as methods using the CRISPR/Cas9 system are known in the art.

### IV. General Pharmaceutical Compositions

In some embodiments, pharmaceutical compositions are administered to a subject. Different aspects may involve administering an effective amount of a composition to a subject. In some embodiments, an antibody or antigen binding fragment capable of binding to TdT may be administered to the subject to protect against or treat a condition (e.g., cancer). Alternatively, an expression vector encoding one or more such antibodies or polypeptides or peptides may be given to a subject as a preventative treatment. Additionally, such compositions can be administered in combination with an additional therapeutic agent (e.g., a chemotherapeutic, an immunotherapeutic, a biotherapeutic, etc.). Such compositions will generally be dissolved or dispersed in a pharmaceutically acceptable carrier or aqueous medium.

The phrases “pharmaceutically acceptable” or “pharmacologically acceptable” refer to molecular entities and compositions that do not produce an adverse, allergic, or other untoward reaction when administered to an animal or human. As used herein, “pharmaceutically acceptable carrier” includes any and all solvents, dispersion media, coatings, anti-bacterial and anti-fungal agents, isotonic and absorption delaying agents, and the like. The use of such media and agents for pharmaceutical active substances is well known in the art. Except insofar as any conventional media or agent is incompatible with the active ingredients, its use in immunogenic and therapeutic compositions is contemplated. Supplementary active ingredients, such as other anti-infective agents and vaccines, can also be incorporated into the compositions.

The active compounds can be formulated for parenteral administration, e.g., formulated for injection via the intravenous, intramuscular, subcutaneous, or intraperitoneal routes. Typically, such compositions can be prepared as either liquid solutions or suspensions; solid forms suitable for use to prepare solutions or suspensions upon the addition of a liquid prior to injection can also be prepared; and, the preparations can also be emulsified.

The pharmaceutical forms suitable for injectable use include sterile aqueous solutions or dispersions; formulations including, for example, aqueous propylene glycol; and sterile powders for the extemporaneous preparation of sterile injectable solutions or dispersions. In all cases the form must be sterile and must be fluid to the extent that it may be easily injected. It also should be stable under the conditions of manufacture and storage and must be preserved against the contaminating action of microorganisms, such as bacteria and fungi.

The proteinaceous compositions may be formulated into a neutral or salt form. Pharmaceutically acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

A pharmaceutical composition can include a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion, and by the use of surfactants. The prevention of the action of microorganisms can be brought about by various anti-bacterial and anti-fungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various other ingredients enumerated above, as required, followed by filtered sterilization or an equivalent procedure. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques, which yield a powder of the active ingredient, plus any additional desired ingredient from a previously sterile-filtered solution thereof.

Administration of the compositions will typically be via any common route. This includes, but is not limited to oral, or intravenous administration. Alternatively, administration may be by orthotopic, intradermal, subcutaneous, intramuscular, intraperitoneal, or intranasal administration. Such compositions would normally be administered as pharmaceutically acceptable compositions that include physiologically acceptable carriers, buffers or other excipients.

Upon formulation, solutions will be administered in a manner compatible with the dosage formulation and in such amount as is therapeutically or prophylactically effective. The formulations are easily administered in a variety of dosage forms, such as the type of injectable solutions described above.

### IX. Kits of the Disclosure

Any of the compositions described herein may be comprised in a kit. In a non-limiting example, any polynucleotides, polypeptides, and/or cells encompassed herein may be comprised in a kit. The kits may alternatively or additionally comprise one or more reagents for generating any polynucleotides, polypeptides, and/or cells encompassed herein, such as primers, deoxynucleotides, buffers, salts, etc.

Any components of the kits may be packaged either in aqueous media or in lyophilized form. The container means of the kits may generally include at least one vial, test tube, flask, bottle, syringe or other container means, into which a component may be placed, and preferably, suitably aliquoted. Where there are more than one component in the kit, the kit also may generally contain a second, third or other additional container into which the additional components may be separately placed. However, various combinations of components may be comprised in a vial. The kits of the present invention also will typically include a means for containing the composition(s) and any other reagent containers in close confinement for commercial sale. Such containers may include injection or blow-molded plastic containers into which the desired vials are retained. In specific embodiments, the kit may comprises one or more apparatuses for taking a biological sample from an individual.

### EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### TdT-Specific Cytotoxic Receptors and Methods of Their Use

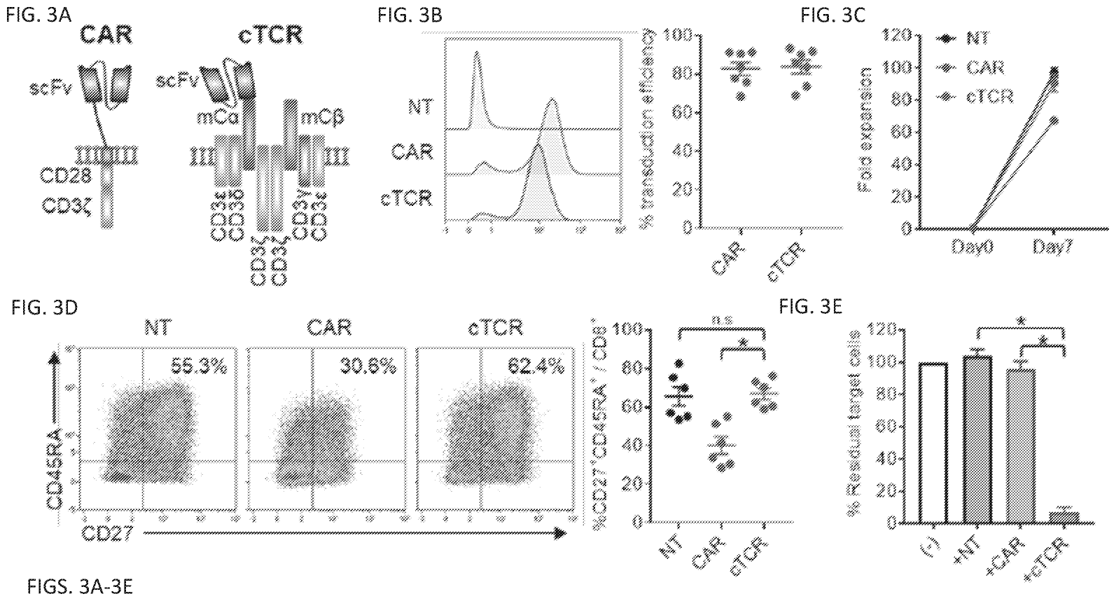

Terminal deoxynucleotidyl transferase (TdT) is a DNA polymerase expressed in T- and B-cell progenitors where it catalyzes transfer of nucleotides during DNA rearrangement^{9,10}. TdT is expressed in up to 95% of T-and B-lineage ALL and is widely used as a diagnostic marker for these malignancies⁴. In addition, TdT expression is frequently detected in minimally differentiated AML^{11,12}. Off-target activity of TdT in immature progenitors has been linked with leukemogenesis^{5,6}. Absence of TdT expression in primitive hematopoietic progenitors or mature peripheral lymphocytes makes it an ideal therapeutic target for leukemia. However, TdT-specific TCRs are effectively deleted in thymus, and intranuclear localization of TdT precludes its targeting with conventional CARs. Therefore, it was considered that TdT-expressing tumor cells can be targeted using a chimeric receptor that recognizes a TdT-derived peptide in the context of MHC I. A meta-analysis was performed of surface peptides presented by TdT+HLAA2+ leukemic cells and a TdT-derived peptide was identified that presented in the context of HLA-A02, a dominant MHC class I allele in the Western world¹³. Using recombinant HLA-A02: 01/TdT peptide complexes, the inventors screened two separate phage display libraries comprised of >2×10¹² humanized scFv and humanized camelid VHH binders. Four clones that recognize TdT/HLA-A02: 01 but not a control peptide/HLA-A02: 01 complex have been identified. Because each individual peptide represents a very small fraction of total HLA-A molecules, surface density of TdT/HLA-A02 complexes is expected to be relatively low, possibly below the threshold of optimal CAR-mediated detection. To address this possibility, for each binder both second-generation TdT-specific CAR and TdT-specific chimeric TCR (cTCR) were generated to take advantage of the modular CAR structure and a highly sensitive TCR signaling, which is capable of detecting only a few cognate peptide/HLA molecules on the cell surface^{14,15} (FIG. 3A). In the cTCR construct, the TdT binders were fused with murine constant TCRα and/or β chains to enable specific dimerization and efficient integration with the CD3 signaling complex in human T-cells (FIG. 3A). Following gammaretroviral transduction, most TdT-specific receptors were expressed on the cell surface of primary human T-cells, with most binder variants demonstrating high level of expression (FIG. 3B) and expansion of transgenic T-cells (FIG. 3C). Expression of the TdT-specific CAR in T-cells resulted in a moderate reduction of minimally differentiated CD27+ CD45RA+ T-cells, likely due to tonic CAR signaling, whereas expression of TdT.cTCR did not affect T-cell differentiation status (FIG. 3D). Cytotoxicity of T-cells expressing TdT-specific constructs was evaluated in a 72-hour coculture with a TdT+ HLA-A02+ leukemia cell line BV173. One TdT-specific cTCR construct produced robust cytotoxicity against leukemic cells whereas other clones exhibited reduced activity compared thereto (FIG. 3E). Notably, T-cells expressing a CAR with an identical TdT binder demonstrated only minimal activity suggesting CAR sensitivity was insufficient to elicit optimal T-cell activation.

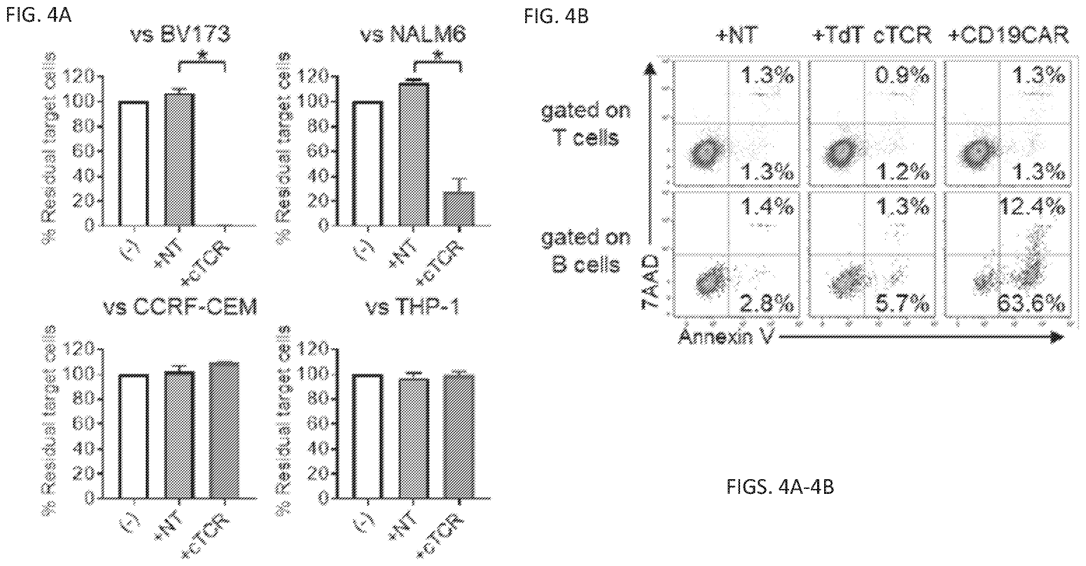

To assess specificity of cTCR T cells, 72-hour coculture assays were performed with malignant and normal lymphocytes and residual target cell number was quantified by flow cytometry with counting beads. TdT.cTCR T-cells produced high cytotoxicity against TdT+/HLA-A2+ BV173 and NALM6 leukemia but demonstrated no activity against negative control CCRF-CEM (TdT+/HLA-A2−) and THP-1 (TdT−/HLA-A2+) cells (FIG. 4A) as well as against TdT−/HLA-A2+ cell lines GDM-1, Jeko-1, and Loucy. Next, TdT.cTCR T-cells were generated from HLA-A02+ donors and performed 24-hour coculture assays with autologous T- and B-cells freshly isolated from peripheral blood. No cytotoxicity against normal T- and B-lymphocytes was observed upon coculture with TdT.cTCR T-cells whereas control CD19 CAR T-cells expectedly killed autologous CD19+ B-cells (FIG. 4B). Expression of TdT.cTCR on HLA-A02+ T-cells also did not produce fratricide and did not affect their ex vivo expansion. These data indicate that TdT.cTCR specifically recognizes TdT peptide in the context of HLA-A02 and does not elicit off-target activity against normal TdT-negative lymphocytes.

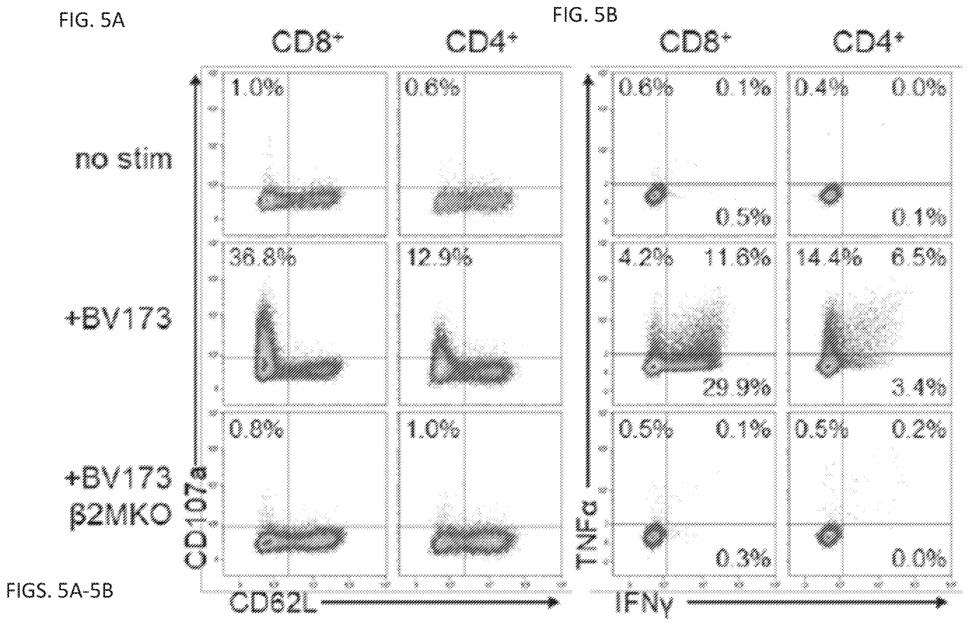

Because the TdT-specific binders identified in the phage display screening recognized TdT/HLA-A02 complexes without the help from the CD8 coreceptor, it was considered that TdT.cTCR would effectively redirect both CD4+ and CD8+ T-cells against leukemia. Indeed, there was robust degranulation (measured by CD107a staining) and production of IFNγ and TNFα by both CD4+ and CD8+ T-cells upon coculture with BV173 target cells (FIG. 5). This activity was abrogated by removing surface MHC class I expression using b2M CRISPR knockout indicating the activity of both CD4+ and CD8+ TdT.cTCR T-cells was MHC class I-specific (FIG. 5). Therefore, unlike conventional tumor antigen-specific TCRs, TdT.cTCR can engage both CD4+ and CD8+ T-cell arms of immune response against leukemia.

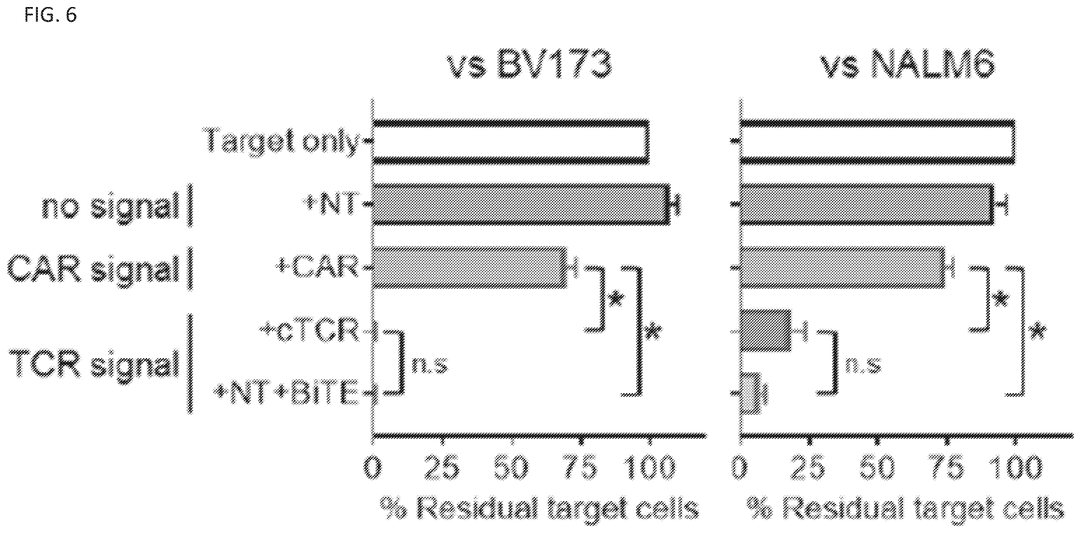

The results above indicate that TdT targeting is most effective by activating conventional TCR signaling. In addition to cTCR-transgenic Tcells, the TCR pathway can be engaged using a soluble bispecific T-cell engager (BiTE) where a tumor antigen-specific binder is fused with an anti-CD3 moiety thus inducing TCR crosslinking and T-cell activation/degranulation against the target cell. To test whether non-modified T-cells can be redirected against TdT+ leukemia with a BiTE, a TdT-specific engager was designed and expressed in producer cells. Standard 72-hour coculture assays were performed of unmodified non-transduced T-cells and TdT.CAR and TdT.cTCR T-cells against TdT+/HLA-A02+ leukemic cell lines BV173 and NALM-6. While control non-transduced T-cells had no measurable activity against leukemia in the absence of the BiTE, addition of soluble BiTE resulted in robust elimination of both leukemia targets by unmodified T-cells, on par with TdT.cTCR T-cells (FIG. 6). Collectively, these results demonstrate strong and specific anti-leukemic activity of TdT-specific cTCR and BiTE constructs compared to the CAR-based modality and support feasibility and efficacy of targeting TdT+ leukemia using these reagents.

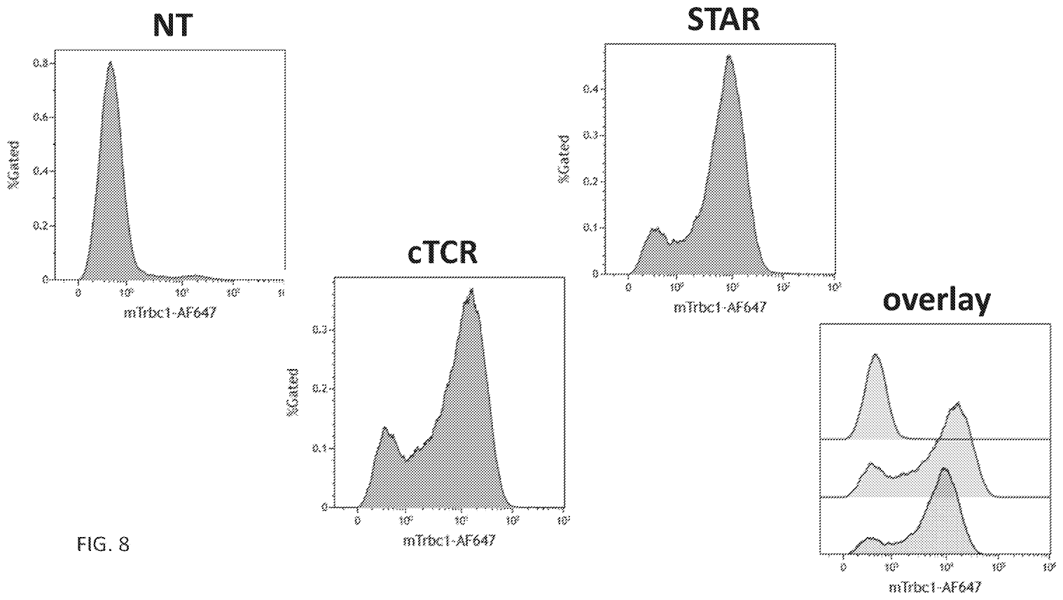

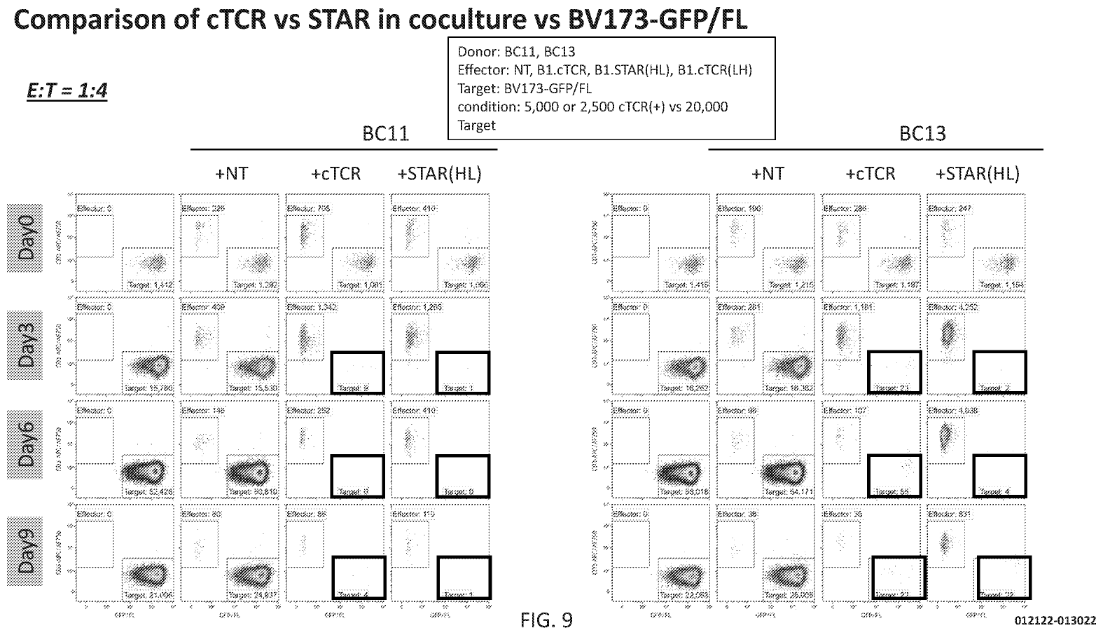

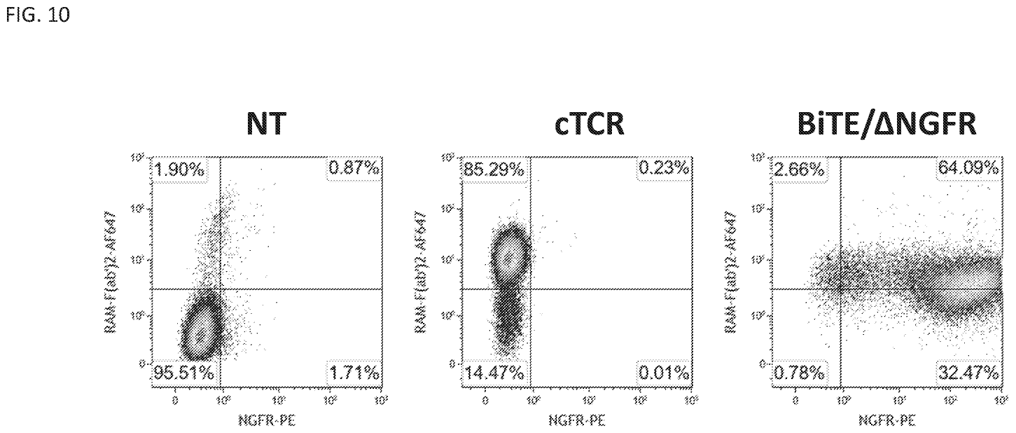

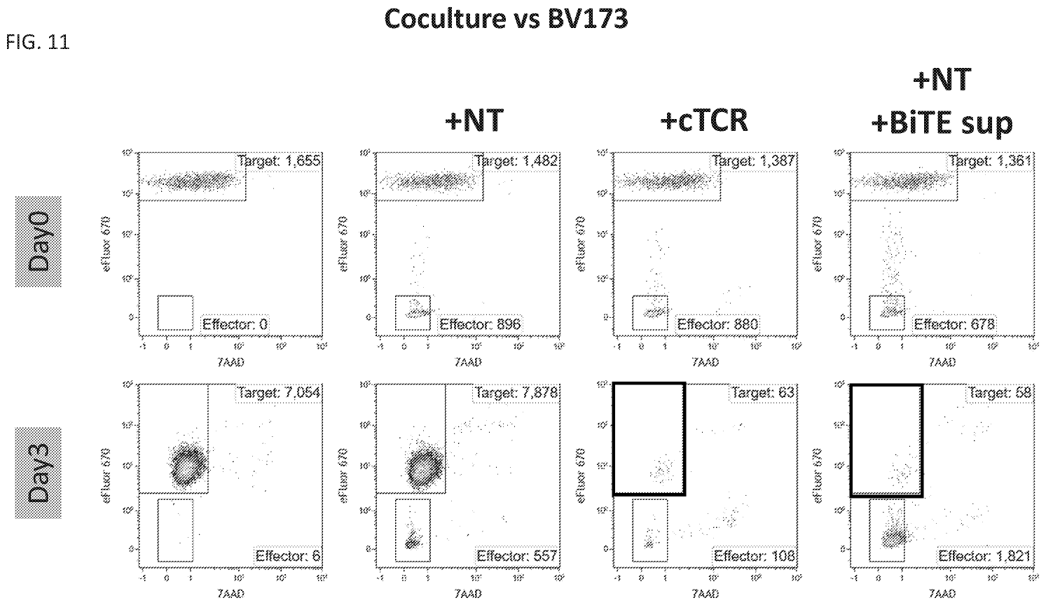

FIG. 7 illustrates one embodiment of a chimeric TCR in which V_{H} and V_{L} domains of the antibody are present on a single TCRα chain, in contrast to STAR receptors comprising the variable heavy and variable light regions on separate chains of a chimeric TCR (Liu et al., Sci Transl Med. 2021; 13(586):eabb5191). FIG. 8 shows cTCR and STAR expression on T cells after retroviral transduction. FIG. 9 demonstrates cytotoxicity of cTCR and STAR-expressing T cells against BV173 cell line (TdT+, HLA-A2+). In particular, the transduced cells were compared in co-culture vs. a representative cancer cell line of mixed-lineage leukemia (MLL) cells. In another embodiment, cTCR and BiTE molecules were transduced retrovirally into T cells (FIG. 10), and FIG. 11 provides evidence of cytotoxicity of the cTCR T cells and BiTE T cells against a representative cancer cell line of MLL cells (TdT+, HLA-A2+).

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
1. Majzner, R. G. & Mackall, C. L. Clinical lessons learned from the first leg of the CAR T cell journey. Nature Medicine 25, 1341-1355 (2019).
2. Lulla, P. D., Mamonkin, M. & Brenner, M. K. Adoptive Cell Therapy for Acute Myeloid Leukemia and T-Cell Acute Lymphoblastic Leukemia. Cancer J 25, 199-207 (2019).
3. Mardiana, S. & Gill, S. CAR T Cells for Acute Myeloid Leukemia: State of the Art and Future Directions. Front Oncol 10, 697 (2020).
4. Drexler, H. G., Menon, M. & Minowada, J. Incidence of TdT positivity in cases of leukemia and lymphoma. Acta Haematol 75, 12-17 (1986).
5. Borrow, J., Dyer, S. A., Akiki, S. & Griffiths, M. J. Molecular roulette: nucleophosmin mutations in AML are orchestrated through N-nucleotide addition by TdT. Blood 134, 2291-2303 (2019).
6. Borrow, J., Dyer, S. A., Akiki, S. & Griffiths, M. J. Terminal deoxynucleotidyl transferase promotes acute myeloid leukemia by priming FLT3-ITD replication slippage. Blood 134, 2281-2290 (2019).
7. Watanabe, K. et al. Target antigen density governs the efficacy of anti-CD20-CD28-CD3 ζ chimeric antigen receptor-modified effector CD8+ T cells. J Immunol 194, 911-920 (2015).
8. Gudipati, V. et al. Inefficient CAR-proximal signaling blunts antigen sensitivity. Nature Immunology 21, 848-856 (2020).
9. Kunkel, T. A., Gopinathan, K. P., Dube, D. K., Snow, E. T. & Loeb, L. A. Rearrangements of DNA mediated by terminal transferase. Proc Natl Acad Sci U S A 83, 1867-1871 (1986).
10. Desiderio, S. V. et al. Insertion of N regions into heavy-chain genes is correlated with expression of terminal deoxytransferase in B cells. Nature 311, 752-755 (1984).
11. Patel, J. L. et al. The immunophenotype of T-lymphoblastic lymphoma in children and adolescents: a Children's Oncology Group report. Br J Haematol 159, 454-461 (2012).
12. Drexler, H. G., Sperling, C. & Ludwig, W. D. Terminal deoxynucleotidyl transferase (TdT) expression in acute myeloid leukemia. Leukemia 7, 1142-1150 (1993).
13. Klatt, M. G. et al. Solving an MHC allele-specific bias in the reported immunopeptidome. JCI Insight 5, (2020).
14. Purbhoo, M. A., Irvine, D. J., Huppa, J. B. & Davis, M. M. T cell killing does not require the formation of a stable mature immunological synapse. Nat Immunol 5, 524-530 (2004).
15. Sykulev, Y., Joo, M., Vturina, I. & Eisen, H. N. Evidence that a single peptide-MHC complex on a target cell can elicit a cytolytic T cell response. Immunity 4, 565-571 (1996).

What is claimed is:

1. A polynucleotide comprising sequence that encodes a recombinant T-cell receptor (TCR) comprising an antibody specific for a terminal deoxynucleotidyl transferase (TdT) or a TdT peptide.

2. The polynucleotide of claim 1, wherein the antibody is a single-chain fragment variable (scFv).

3. The polynucleotide of claim 1 or 2, wherein the TdT peptide is a TdT peptide associated with HLA-A02 on cell surfaces.

4. The polynucleotide of any one of claims 1-3, wherein the TdT peptide comprises SEQ ID NO:3 or a functional variant thereof comprising at least 80% identity to SEQ ID NO:3.

5. The polynucleotide of any one of the preceding claims, wherein a TCRα chain and/or TCRβ chain of the TCR have one or more modifications to prevent cross-pairing with endogenous TCR when present in a cell.

6. The polynucleotide of any one of the preceding claims, wherein a TCRα chain and/or TCRβ chain of the TCR have one or more modifications to enhance heterologous pairing with each other.

7. The polynucleotide of any one of the preceding claims, wherein the TCR is further defined as comprising murine constant TCRα chain, murine constant TCRβ chain, or both.

8. The polynucleotide of any one of claims 1-7, wherein both variable light chain (V_{L}) and variable heavy chain (V_{H}) domains of the antibody are on the same TCR chain.

9. The polynucleotide of any one of claims 1-5, wherein one TCR chain is attached to the V_{H} domain and another TCR chain is attached to the V_{L} chain.

10. The polynucleotide of any one of the preceding claims, wherein a TCR chain comprises the sequence of SEQ ID NO:1 or SEQ ID NO:2.

11. The polynucleotide of any one of the preceding claims, wherein the scFv comprises SEQ ID NO:5, 7, or 9 or is encoded by SEQ ID NO: 4, 6, or 8.

12. The polynucleotide of any one of the preceding claims, wherein the polynucleotide further comprises sequence that encodes a bispecific T-cell engager (BiTE).

13. The polynucleotide of claim 12, wherein the BiTE comprises an antibody specific for TdT or a TdT peptide.

14. The polynucleotide of any one of the preceding claims, wherein the recombinant TCR comprises SEQ ID NO: 11, 13, 15, or 17 or is encoded by SEQ ID NO: 10, 12, 14, or 16.

15. The polynucleotide of any one of claims 12-14, wherein the BiTE comprises SEQ ID NO:19 or is encoded by SEQ ID NO:18.

16. A polynucleotide comprising sequence that encodes a bispecific antibody comprising an antigen binding region specific for TdT peptide.

17. The polynucleotide of claim 16, wherein the bispecific antibody comprises an antigen binding region specific for CD3.

18. The polynucleotide of any one of claim 18 or 19, wherein the sequence comprises SEQ ID NO:18 or encodes SEQ ID NO:19.

19. A composition, comprising the polynucleotide of any one of the preceding claims.

20. The composition of claim 19, wherein the composition comprises a vector.

21. The composition of claim 20, wherein the vector is a viral vector or a non-viral vector.

22. The composition of claim 21, wherein the viral vector is an adenoviral vector, adeno-associated viral vector, retroviral vector, or lentiviral vector.

23. The composition of claim 21, wherein the non-viral vector is a plasmid or transposon.

24. A polypeptide encoded by the polynucleotide of any one of claims 1-18 or a composition of any one of claims 19-23.

25. A cell, comprising the polynucleotide of any one of claims 1-18 or a composition of any one of claims 19-23.

26. The cell of claim 25, wherein the cell is an immune cell.

27. The cell of claim 25 or 26, wherein the cell is a T cell, NK cell, NK T cell, B cell, macrophage, neutrophil, or a combination thereof.

28. The cell of claim 27, wherein the T cell is an ab T cell, gd T cell, T-helper cells, invariant natural killer T (iNKT) cells, cytotoxic T cells, T-regulatory cells natural-killer (NK) cells, or a combination thereof.

29. The cell of any one of claims 25-28, further defined as comprising:
a polynucleotide comprising (1) sequence that encodes a recombinant TCR comprising an scFv specific for TdT or a TdT peptide; and (2) sequence that encodes a BiTE comprising an antibody specific for TdT or a TdT peptide; or
a first polynucleotide comprising sequence that encodes a recombinant TCR comprising an scFv specific for TdT or a TdT peptide; and a second polynucleotide comprising sequence that encodes a BiTE comprising an antibody specific for TdT or a TdT peptide.

30. The cell of claim 29, wherein the variable light chain (V_{L}) and variable heavy chain (V_{H}) domains of the antibody are on the same TCR chain.

31. The cell of claim 29, wherein one TCR chain is attached to the V_{H} domain and another TCR chain is attached to the V_{L} domain.

32. The cell of any one of claims 25-31, wherein the cell is a T cell.

33. A method of treating a medical condition in an individual caused indirectly or directly by the presence of TdT or a TdT peptide associated with HLA-A02 on the surface of cells in the individual, comprising the step of administering a therapeutically effective amount of the composition of any one of claims 19-23 and/or a therapeutically effective amount of the cells of any one of claims 25-32 to the individual.

34. The method of claim 33, wherein the medical condition is cancer.

35. The method of claim 34, wherein the cancer comprises a hematological malignancy.

36. The method of claim 34, wherein the cancer comprises a solid tumor.

37. The method of claim 35, wherein the hematological malignancy is leukemia or lymphoma.

38. The method of claim 37, wherein the leukemia is acute myeloid leukemia (AML), chronic myeloid (or myelogenous) leukemia (CML); acute lymphocytic (or lymphoblastic) leukemia (ALL); or chronic lymphocytic leukemia.

39. The method of claim 37, wherein the lymphoma is Hodgkin lymphoma, non-Hodgkin lymphoma, chronic lymphocytic leukemia (CLL), or small lymphocytic lymphoma (SLL).

40. The method of any one of claims 33-39, wherein the medical condition is cancer and wherein the individual is administered a therapeutically effective amount of cells comprising recombinant TCR comprising an scFv specific a TdT peptide and wherein the individual is administered a therapeutically effective amount of cells comprising a BiTE comprising an scFv specific for a TdT peptide.

41. The method of claim 40, wherein the cells comprising the recombinant TCR and the cells comprising the BiTE are the same cells.

42. The method of claim 40, wherein the cells comprising the recombinant TCR and the cells comprising the BiTE are different cells.

43. A kit, housed in a suitable container, comprising the polynucleotide of any one of claims 1-18, a composition of any one of claims 19-23, and/or cells of any one of claims 25-32.


SEQUENCE LISTING (45 sequences)

SEQ ID NO: 1 - protein, 138 aa, synthetic construct
PYIQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLLVIVLRILLLKVAGFNLLMTLRLWSS

SEQ ID NO: 2 - protein, 173 aa, synthetic construct
EDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 3 - protein, 9 aa, synthetic construct
ALYDKTKRI

SEQ ID NO: 4 - DNA, 789 bp, synthetic construct
  source 1..789: mol_type = other DNA
atggccttaccagtgaccgccttgctcctgccgctggccttgctgctccacgccgccaggccggctcaagttcagctggtgcagagcggagcggaggtgaagaagccagggtcttccgttaaggtctcttgtaaggcctcaggcggaactttctcatcttacgctatttcctgggtaaggcaagcaccaggacagggattagaatggatgggggggattatccccattttcggtaccgcgaactacgcccaaaaatttcaaggtcgggtaaccatcaccgccgatgaatcgacatctaccgcctacatggaactgagctctcttcggagcgaggacaccgcagtctattactgcgcgagagacgggtatagtggtagctactactattattacggcatggacgtttggggccaggggaccttagtgacggtgagcagcggaggaggcggcgggtcccagtctgctctgacccaacctgccagcgtctccggcagtccaggacaaagtatcaccatcagctgcactgggacttccagcgatgtggggggctacaattacgtaagttggtaccagcaacaccctgggaaggcccccaagctgatgatttatgatgtctcatacagaccgagcggagtgagccaccgttttagtggctccaagtccggaaacaccgcttcactaacaatcagtggcttacaagcagaggacgaagccgattattactgctcatcttacaccagttccagtacgcttgtgtttggtactggaacaaagttgactgtgctgggc

SEQ ID NO: 5 - protein, 262 aa, synthetic construct
MALPVTALLLPLALLLHAARPAQVQLVQSGAEVKKPGSSVKVSCKASGGTFSSYAISWVRQAPGQGLEWMGGIIPIFGTANYAQKFQGRVTITADESTSTAYMELSSLRSEDTAVYYCARDGYSGSYYYYYGMDVWGQGTLVTVSSGGGGGSQSALTQPASVSGSPGQSITISCTGTSSDVGGYNYVSWYQQHPGKAPKLMIYDVSYRPSGVSHRFSGSKSGNTASLTISGLQAEDEADYYCSSYTSSSTLVFGTGTKLTVL

SEQ ID NO: 6 - DNA, 801 bp, synthetic construct
  source 1..801: mol_type = other DNA
atggccttaccagtgaccgccttgctcctgccgctggccttgctgctccacgccgccaggccggcccaagtccagctggttgaaagcgggggaggagtggttcagcccggaaggagtttgcggttgagttgtgcagcatcaggattcacattttcttcctatgcaatgtcctgggtgcgtcaggctcccggaaagggactggaatgggttagcgctatatctggcagcggagactctacatattatgctgattcagtcaaaggccgtttcaccatcagcagagacaacagtaagaataccctctaccttcagatgaactcgctacgtgctgaagacaccgccgtttactactgtgctaaagatctcgactcctcaagtccggacgatgcttttgacatctggggacaaggcaccactgtcaccgtcagcagcggaggcgccgcgctggccgaagtggcagccgcggtggctgaccctatcgttctgactcagtctcctggaaagctgagcctctccccaggggagcgggccactttgtcctgtggcgcgtcgcagagcgtatcatccaattacctggcatggtaccagcagaaacctggcctggctcctcggcttttgatttatgatgctagtattagggctactggcgtgcctgatcggttctcgggatcagggagcgcgactgacttcactttgacgatcagccggctggaccctgaggattttgcggtgtactattgccaccagtattcatctgcccccatgacttttggacagggcactaagctggagattaagagg

SEQ ID NO: 7 - protein, 267 aa, synthetic construct
MALPVTALLLPLALLLHAARPAQVQLVESGGGVVQPGRSLRLSCAASGFTFSSYAMSWVRQAPGKGLEWVSAISGSGDSTYYADSVKGRFTISRDNSKNTLYLQMNSLRAEDTAVYYCAKDLDSSSPDDAFDIWGQGTTVTVSSGGAALAEVAAAVADPIVLTQSPGKLSLSPGERATLSCGASQSVSSNYLAWYQQKPGLAPRLLIYDASIRATGVPDRFSGSGSATDFTLTISRLDPEDFAVYYCHQYSSAPMTFGQGTKLEIKR

SEQ ID NO: 8 - DNA, 783 bp, synthetic construct
  source 1..783: mol_type = other DNA
atggccttaccagtgaccgccttgctcctgccgctggccttgctgctccacgccgccaggccggctcaagtgcagctggtcgaatctggagctgggactaagaagcctggcgagagtctgaagatctcttgtaaggccagcggctacaactttgcttcttactggatcgggtgggttagacagatgcctggaaaaggacttgagtgggtagggatcattgatcctagcgattccgataccaaatacagcccaagctttgaagggcaagtcactattagtgtggacaagtctatcagcactgctcacctgcagtggagtagcctagatgcgagcgacacggccatgtactactgcgcgaggagtctgggcagctactatggagattggtatttcgatctctgggggaggggcacactggtgaccgtgtcatccggaggaggcggcgggtccaactttatgctgacccagccacattcaatgtctgaaagccctggcaaaaccgtaacaatctcgtgcacacggtcaagcgggagcatcgccaacaactatgttcagtggtatcagcagagacccgggagttcccctaccactgtcatctacgatgacaatcagagaccatccggggtgcccgaccgcttcagcggctcaatagatagctccagcaactccgcctctttaacaatctcgggcctgaaaaccgaggacgaagctgattattactgccagtcctatgactccagcaacgtgatttttggtggagggactaaactgaccgtcctcgga

SEQ ID NO: 9 - protein, 261 aa, synthetic construct
MALPVTALLLPLALLLHAARPAQVQLVESGAGTKKPGESLKISCKASGYNFASYWIGWVRQMPGKGLEWVGIIDPSDSDTKYSPSFEGQVTISVDKSISTAHLQWSSLDASDTAMYYCARSLGSYYGDWYFDLWGRGTLVTVSSGGGGGSNFMLTQPHSMSESPGKTVTISCTRSSGSIANNYVQWYQQRPGSSPTTVIYDDNQRPSGVPDRFSGSIDSSSNSASLTISGLKTEDEADYYCQSYDSSNVIFGGGTKLTVLG

SEQ ID NO: 10 - DNA, 858 bp, synthetic construct
  source 1..858: mol_type = other DNA
atggccttaccagtgaccgccttgctcctgccgctggccttgctgctccacgccgccaggccggctcaagttcagctggtgcagagcggagcggaggtgaagaagccagggtcttccgttaaggtctcttgtaaggcctcaggcggaactttctcatcttacgctatttcctgggtaaggcaagcaccaggacagggattagaatggatgggggggattatccccattttcggtaccgcgaactacgcccaaaaatttcaaggtcgggtaaccatcaccgccgatgaatcgacatctaccgcctacatggaactgagctctcttcggagcgaggacaccgcagtctattactgcgcgagagacgggtatagtggtagctactactattattacggcatggacgtttggggccaggggaccttagtgacggtgagcagcggatccccatacattcaaaatccagaacctgcggtataccaattgaaagacccaagaagccaggattccacgctgtgtctgtttacagacttcgactcacagatcaatgttcccaagacaatggaaagcgggacgttcatcaccgataaatgcgttctggacatgaaggctatggattctaaaagtaatggtgctatagcctggagtaaccagacctcatttacttgccaagacattttcaaggagaccaatgctacatatccatcctccgacgtcccttgtgacgccactctgaccgagaaatcttttgagaccgatatgaacctgaactttcagaacctactggttatcgttctgagaatactgttgctcaaagtcgccggattcaaccttctcatgactctgagactgtggagtagc

SEQ ID NO: 11 - protein, 286 aa, synthetic construct
MALPVTALLLPLALLLHAARPAQVQLVQSGAEVKKPGSSVKVSCKASGGTFSSYAISWVRQAPGQGLEWMGGIIPIFGTANYAQKFQGRVTITADESTSTAYMELSSLRSEDTAVYYCARDGYSGSYYYYYGMDVWGQGTLVTVSSGSPYIQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLLVIVLRILLLKVAGFNLLMTLRLWSS

SEQ ID NO: 12 - DNA, 921 bp, synthetic construct
  source 1..921: mol_type = other DNA
atggagacagacacactcctgctatgggtgctgctgctctgggttccaggttccacaggtcagtctgctctgacccaacctgccagcgtctccggcagtccaggacaaagtatcaccatcagctgcactgggacttccagcgatgtggggggctacaattacgtaagttggtaccagcaacaccctgggaaggcccccaagctgatgatttatgatgtctcatacagaccgagcggagtgagccaccgttttagtggctccaagtccggaaacaccgcttcactaacaatcagtggcttacaagcagaggacgaagccgattattactgctcatcttacaccagttccagtacgcttgtgtttggtactggaacaaagttgactgtgctgggcacgcgtgaggacttgaggaatgtgacgccaccgaaggtctctttgtttgagccgtctaaggcagaaattgctaataaacaaaaggccaccctagtgtgtctcgcgaggggattctttcctgatcacgttgagttgtcctggtgggtgaatggtaaggaggttcatagcggtgtctgcaccgacccccaggcatacaaggagtcgaactactcctactgcctgtcatctagactccgagtcagtgccacgttctggcataacccccgtaaccattttaggtgccaggtgcaattccacgggcttagtgaagaggacaagtggccagaggggtccccaaagccggtgacccaaaatatctctgcggaagcttgggggcgcgccgattgtgggattaccagcgcgagctatcagcagggagtcctgagtgcaacgatactctacgagatattactgggaaaggctacattatatgcagtgttggtgagcactctggtcgtcatggcaatggttaagagaaagaatagctaa

SEQ ID NO: 13 - protein, 306 aa, synthetic construct
METDTLLLWVLLLWVPGSTGQSALTQPASVSGSPGQSITISCTGTSSDVGGYNYVSWYQQHPGKAPKLMIYDVSYRPSGVSHRFSGSKSGNTASLTISGLQAEDEADYYCSSYTSSSTLVFGTGTKLTVLGTREDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 14 - DNA, 1209 bp, synthetic construct
  source 1..1209: mol_type = other DNA
atggccttaccagtgaccgccttgctcctgccgctggccttgctgctccacgccgccaggccggctcaagttcagctggtgcagagcggagcggaggtgaagaagccagggtcttccgttaaggtctcttgtaaggcctcaggcggaactttctcatcttacgctatttcctgggtaaggcaagcaccaggacagggattagaatggatgggggggattatccccattttcggtaccgcgaactacgcccaaaaatttcaaggtcgggtaaccatcaccgccgatgaatcgacatctaccgcctacatggaactgagctctcttcggagcgaggacaccgcagtctattactgcgcgagagacgggtatagtggtagctactactattattacggcatggacgtttggggccaggggaccttagtgacggtgagcagcggaggaggcggcgggtcccagtctgctctgacccaacctgccagcgtctccggcagtccaggacaaagtatcaccatcagctgcactgggacttccagcgatgtggggggctacaattacgtaagttggtaccagcaacaccctgggaaggcccccaagctgatgatttatgatgtctcatacagaccgagcggagtgagccaccgttttagtggctccaagtccggaaacaccgcttcactaacaatcagtggcttacaagcagaggacgaagccgattattactgctcatcttacaccagttccagtacgcttgtgtttggtactggaacaaagttgactgtgctgggcggatccccatacattcaaaatccagaacctgcggtataccaattgaaagacccaagaagccaggattccacgctgtgtctgtttacagacttcgactcacagatcaatgttcccaagacaatggaaagcgggacgttcatcaccgataaatgcgttctggacatgaaggctatggattctaaaagtaatggtgctatagcctggagtaaccagacctcatttacttgccaagacattttcaaggagaccaatgctacatatccatcctccgacgtcccttgtgacgccactctgaccgagaaatcttttgagaccgatatgaacctgaactttcagaacctactggttatcgttctgagaatactgttgctcaaagtcgccggattcaaccttctcatgactctgagactgtggagtagc

SEQ ID NO: 15 - protein, 403 aa, synthetic construct
MALPVTALLLPLALLLHAARPAQVQLVQSGAEVKKPGSSVKVSCKASGGTFSSYAISWVRQAPGQGLEWMGGIIPIFGTANYAQKFQGRVTITADESTSTAYMELSSLRSEDTAVYYCARDGYSGSYYYYYGMDVWGQGTLVTVSSGGGGGSQSALTQPASVSGSPGQSITISCTGTSSDVGGYNYVSWYQQHPGKAPKLMIYDVSYRPSGVSHRFSGSKSGNTASLTISGLQAEDEADYYCSSYTSSSTLVFGTGTKLTVLGGSPYIQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLLVIVLRILLLKVAGFNLLMTLRLWSS

SEQ ID NO: 16 - DNA, 1323 bp, synthetic construct
  source 1..1323: mol_type = other DNA
atggccttaccagtgaccgccttgctcctgccgctggccttgctgctccacgccgccaggccggctcaagttcagctggtgcagagcggagcggaggtgaagaagccagggtcttccgttaaggtctcttgtaaggcctcaggcggaactttctcatcttacgctatttcctgggtaaggcaagcaccaggacagggattagaatggatgggggggattatccccattttcggtaccgcgaactacgcccaaaaatttcaaggtcgggtaaccatcaccgccgatgaatcgacatctaccgcctacatggaactgagctctcttcggagcgaggacaccgcagtctattactgcgcgagagacgggtatagtggtagctactactattattacggcatggacgtttggggccaggggaccttagtgacggtgagcagcggaggaggcggcgggtcccagtctgctctgacccaacctgccagcgtctccggcagtccaggacaaagtatcaccatcagctgcactgggacttccagcgatgtggggggctacaattacgtaagttggtaccagcaacaccctgggaaggcccccaagctgatgatttatgatgtctcatacagaccgagcggagtgagccaccgttttagtggctccaagtccggaaacaccgcttcactaacaatcagtggcttacaagcagaggacgaagccgattattactgctcatcttacaccagttccagtacgcttgtgtttggtactggaacaaagttgactgtgctgggcggatccacgcgtgaggacttgaggaatgtgacgccaccgaaggtctctttgtttgagccgtctaaggcagaaattgctaataaacaaaaggccaccctagtgtgtctcgcgaggggattctttcctgatcacgttgagttgtcctggtgggtgaatggtaaggaggttcatagcggtgtctgcaccgacccccaggcatacaaggagtcgaactactcctactgcctgtcatctagactccgagtcagtgccacgttctggcataacccccgtaaccattttaggtgccaggtgcaattccacgggcttagtgaagaggacaagtggccagaggggtccccaaagccggtgacccaaaatatctctgcggaagcttgggggcgcgccgattgtgggattaccagcgcgagctatcagcagggagtcctgagtgcaacgatactctacgagatattactgggaaaggctacattatatgcagtgttggtgagcactctggtcgtcatggcaatggttaagagaaagaatagctaa

SEQ ID NO: 17 - protein, 440 aa, synthetic construct
MALPVTALLLPLALLLHAARPAQVQLVQSGAEVKKPGSSVKVSCKASGGTFSSYAISWVRQAPGQGLEWMGGIIPIFGTANYAQKFQGRVTITADESTSTAYMELSSLRSEDTAVYYCARDGYSGSYYYYYGMDVWGQGTLVTVSSGGGGGSQSALTQPASVSGSPGQSITISCTGTSSDVGGYNYVSWYQQHPGKAPKLMIYDVSYRPSGVSHRFSGSKSGNTASLTISGLQAEDEADYYCSSYTSSSTLVFGTGTKLTVLGGSTREDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 18 - DNA, 1527 bp, synthetic construct
  source 1..1527: mol_type = other DNA
atggattggatctggcgcatcctgtttctcgtgggagccgccacaggcgcccattctgctcaagttcagctggtgcagagcggagcggaggtgaagaagccagggtcttccgttaaggtctcttgtaaggcctcaggcggaactttctcatcttacgctatttcctgggtaaggcaagcaccaggacagggattagaatggatgggggggattatccccattttcggtaccgcgaactacgcccaaaaatttcaaggtcgggtaaccatcaccgccgatgaatcgacatctaccgcctacatggaactgagctctcttcggagcgaggacaccgcagtctattactgcgcgagagacgggtatagtggtagctactactattattacggcatggacgtttggggccaggggaccttagtgacggtgagcagcggaggaggcggcgggtcccagtctgctctgacccaacctgccagcgtctccggcagtccaggacaaagtatcaccatcagctgcactgggacttccagcgatgtggggggctacaattacgtaagttggtaccagcaacaccctgggaaggcccccaagctgatgatttatgatgtctcatacagaccgagcggagtgagccaccgttttagtggctccaagtccggaaacaccgcttcactaacaatcagtggcttacaagcagaggacgaagccgattattactgctcatcttacaccagttccagtacgcttgtgtttggtactggaacaaagttgactgtgctgggctccggaggtggtggatccgatatcaaactgcagcagtcaggggctgaactggcaagacctggggcctcagtgaagatgtcctgcaagacttctggctacacctttactaggtacacgatgcactgggtaaaacagaggcctggacagggtctggaatggattggatacattaatcctagccgtggttatactaattacaatcagaagttcaaggacaaggccacattgactacagacaaatcctccagcacagcctacatgcaactgagcagcctgacatctgaggactctgcagtctattactgtgcaagatattatgatgatcattactgccttgactactggggccaaggcaccactctcacagtctcctcaggtggtggtggttctggcggcggcggctccggtggtggtggttctgacattcagctgacccagtctccagcaatcatgtctgcatctccaggggagaaggtcaccatgacctgcagagccagttcaagtgtaagttacatgaactggtaccagcagaagtcaggcacctcccccaaaagatggatttatgacacatccaaagtggcttctggagtcccttatcgcttcagtggcagtgggtctgggacctcatactctctcacaatcagcagcatggaggctgaagatgctgccacttattactgccaacagtggagtagtaacccgctcacgttcggtgctgggaccaagctggagctgaaatcctaa

SEQ ID NO: 19 - protein, 508 aa, synthetic construct
MDWIWRILFLVGAATGAHSAQVQLVQSGAEVKKPGSSVKVSCKASGGTFSSYAISWVRQAPGQGLEWMGGIIPIFGTANYAQKFQGRVTITADESTSTAYMELSSLRSEDTAVYYCARDGYSGSYYYYYGMDVWGQGTLVTVSSGGGGGSQSALTQPASVSGSPGQSITISCTGTSSDVGGYNYVSWYQQHPGKAPKLMIYDVSYRPSGVSHRFSGSKSGNTASLTISGLQAEDEADYYCSSYTSSSTLVFGTGTKLTVLGSGGGGSDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSGGGGSGGGGSGGGGSDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKS

SEQ ID NO: 20 - DNA, 648 bp, synthetic construct
  source 1..648: mol_type = other DNA
atggagacagacacactcctgctatgggtgctgctgctctgggttccaggttccacaggtggcggcggtgggtcagaacaaaaattgatttcagaggaagatctcggagggggagggtcaacgcgtgaggacttgaggaatgtgacgccaccgaaggtctctttgtttgagccgtctaaggcagaaattgctaataaacaaaaggccaccctagtgtgtctcgcgaggggattctttcctgatcacgttgagttgtcctggtgggtgaatggtaaggaggttcatagcggtgtctgcaccgacccccaggcatacaaggagtcgaactactcctactgcctgtcatctagactccgagtcagtgccacgttctggcataacccccgtaaccattttaggtgccaggtgcaattccacgggcttagtgaagaggacaagtggccagaggggtccccaaagccggtgacccaaaatatctctgcggaagcttgggggcgcgccgattgtgggattaccagcgcgagctatcagcagggagtcctgagtgcaacgatactctacgagatattactgggaaaggctacattatatgcagtgttggtgagcactctggtcgtcatggcaatggttaagagaaagaatagctaa

SEQ ID NO: 21 - protein, 215 aa, synthetic construct
METDTLLLWVLLLWVPGSTGGGGGSEQKLISEEDLGGGGSTREDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 22 - protein, 125 aa, synthetic construct
AQVQLVQSGAEVKKPGSSVKVSCKASGGTFSSYAISWVRQAPGQGLEWMGGIIPIFGTANYAQKFQGRVTITADESTSTAYMELSSLRSEDTAVYYCARDGYSGSYYYYYGMDVWGQGTLVTVSS

SEQ ID NO: 23 - protein, 123 aa, synthetic construct
AQVQLVESGGGVVQPGRSLRLSCAASGFTFSSYAMSWVRQAPGKGLEWVSAISGSGDSTYYADSVKGRFTISRDNSKNTLYLQMNSLRAEDTAVYYCAKDLDSSSPDDAFDIWGQGTTVTVSS

SEQ ID NO: 24 - protein, 123 aa, synthetic construct
AQVQLVESGAGTKKPGESLKISCKASGYNFASYWIGWVRQMPGKGLEWVGIIDPSDSDTKYSPSFEGQVTISVDKSISTAHLQWSSLDASDTAMYYCARSLGSYYGDWYFDLWGRGTLVTVSS

SEQ ID NO: 25 - protein, 110 aa, synthetic construct
QSALTQPASVSGSPGQSITISCTGTSSDVGGYNYVSWYQQHPGKAPKLMIYDVSYRPSGVSHRFSGSKSGNTASLTISGLQAEDEADYYCSSYTSSSTLVFGTGTKLTVL

SEQ ID NO: 26 - protein, 121 aa, synthetic construct
AALAEVAAAVADPIVLTQSPGKLSLSPGERATLSCGASQSVSSNYLAWYQQKPGLAPRLLIYDASIRATGVPDRFSGSGSATDFTLTISRLDPEDFAVYYCHQYSSAPMTFGQGTKLEIKR

SEQ ID NO: 27 - protein, 111 aa, synthetic construct
NFMLTQPHSMSESPGKTVTISCTRSSGSIANNYVQWYQQRPGSSPTTVIYDDNQRPSGVPDRFSGSIDSSSNSASLTISGLKTEDEADYYCQSYDSSNVIFGGGTKLTVLG

SEQ ID NO: 28 - protein, 8 aa, synthetic construct
GGTFSSYA

SEQ ID NO: 29 - protein, 8 aa, synthetic construct
IIPIFGTA

SEQ ID NO: 30 - protein, 17 aa, synthetic construct
ARDGYSGSYYYYYGMDV

SEQ ID NO: 31 - protein, 9 aa, synthetic construct
SSDVGGYNY

SEQ ID NO: 32 - skipped

SEQ ID NO: 33 - protein, 10 aa, synthetic construct
SSYTSSSTLV

SEQ ID NO: 34 - protein, 8 aa, synthetic construct
GFTFSSYA

SEQ ID NO: 35 - protein, 8 aa, synthetic construct
ISGSGDST

SEQ ID NO: 36 - protein, 15 aa, synthetic construct
AKDLDSSSPDDAFDI

SEQ ID NO: 37 - protein, 7 aa, synthetic construct
QSVSSNY

SEQ ID NO: 38 - skipped

SEQ ID NO: 39 - protein, 9 aa, synthetic construct
HQYSSAPMT

SEQ ID NO: 40 - protein, 8 aa, synthetic construct
GYNFASYW

SEQ ID NO: 41 - protein, 8 aa, synthetic construct
IDPSDSDT

SEQ ID NO: 42 - protein, 15 aa, synthetic construct
ARSLGSYYGDWYFDL

SEQ ID NO: 43 - protein, 8 aa, synthetic construct
SGSIANNY

SEQ ID NO: 44 - skipped

SEQ ID NO: 45 - protein, 9 aa, synthetic construct
QSYDSSNVI